(19) United States
(12) Patent Application Publication
(10) Pub. No.: US 2004/0086511 A1
(43) Pub. Date: May 6, 2004

(54) Neuronal gene expression patterns

(75) Inventors: Donald Jeffery Zack, Baltimore, MD (US); Masaaki Kageyama, Osaka (JP)

Correspondence Address: BANNER & WITCOFF, 1001 G STREET N W, SUITE 1100, WASHINGTON, DC, 20001, US

(73) Assignee: The Johns Hopkins University, Baltimore, MD

(21) Appl. No.: 10/617,885

(22) Filed: Jul. 14, 2003

Related U.S. Application Data
(60) Provisional application No. 60/395,753, filed on Jul. 12, 2002.

(51) Int. Cl. A61K 39/395

(52) U.S. Cl. 424/146.1

(57) ABSTRACT

Neuronal cell death, as modeled by removal of serum or NGF from growth medium, is characterized by many changes in gene expression. Gene expression was compared before and after withdrawal of serum or NGF. These results provide clues to underlying molecular processes occurring during neuronal and photoreceptor degeneration, and provide direction for future cell-based studies.

This application claims the benefit of provisional application serial No. 60/395,753 filed Jul. 12, 2002, the disclosure of which is expressly incorporated herein.

### TECHNICAL FIELD OF THE INVENTION

This invention is related to the area of neuronal cell death. In particular, it relates to genes which are characteristically dyregulated in neuronal cells, including retinal cells, which are subjected to a lethal challenge.

### BACKGROUND OF THE INVENTION

Neuronal cell death is a major feature of a variety of human neurological disorders, including the neurodegenerative diseases (such as Alzheimer's, Parkinson's, Huntington's and amyotrophic lateral sclerosis), stroke and trauma. Alzheimer's Disease afflicts about 4 million people in the United States, primarily the elderly. It is characterized by progressive memory loss, disorientation, depression and eventual loss of bodily functions. Amyotrophic lateral sclerosis, afflicts about 30,000 Americans. It begins after age 40 and results in progressive weakness and paralysis. Huntington's Disease, which afflicts an estimated 25,000 patients in the United States, usually begins between the ages of 30 and 50 and includes violent, involuntary movements.

The molecular events contributing to retinal disease are highly complex, reflecting the interdependence among the different cell types in the retina and the intimate cellular contacts between photoreceptors and the surrounding epithelium. Mutations in over 80 genes have been implicated in retinal degenerations and over 50 additional disease loci have been mapped (RetNet, URL address: www host server, sph.uth.tmc.edu domain name, Retnet/home.htm directory). Many of the disease-causing genes have been identified as key players in retina function, including enzymes involved in phototransduction and the visual cycle, transcription factors that control retina-specific gene expression, and structural proteins that support the unique disk membrane structure of photoreceptors (TINS review). Despite knowledge of the functions of many of these proteins, the cellular and molecular pathways leading from the primary mutation to photoreceptor death are not well understood.

The recent completion of genome sequencing efforts and the accumulation of tissue libraries and EST databases has resulted in dramatic advances in the ability to rapidly characterize gene expression. A number of recent studies have used various gene profiling methodologies to identify novel retina genes and to investigate the pathogenesis of retina disease {Swaroop and Zack, 2002; Wilson 2002}. These methods differ in their application. For example, two techniques, data mining of cDNA libraries {Sinha 2000; Wistow, G. Bernstein, S. L., 2002; Stoehr 2002; Katsanis 2002} and SAGE (serial analysis of gene expression) {Blackshaw et al. 2001}, are highly suited for quantitation-of gene expression under a single experimental state. In comparison, microarray technologies have the advantage of being able to measure gene expression changes across multiple experimental conditions or different disease states. Several types of microarrays, including oligonucleotide and cDNA, custom and commercial, glass and membrane, have been used in investigations of retina disease {Farjo 2002; Livesay 2000; Kennan 2002; June 2001; Joussen 2001; Buraczynska 2002}. Important insights into pathogenic pathways have been gained from such studies, and in at least one example, a new retina disease-causing gene was identified using microarray analysis {Kennan, 2002}.

One aspect arising from the microarray analyses above has been the recognition that custom cDNA and commercial oligonucleotide microarrays have different strengths. Although commercial arrays typically have an order of magnitude more genes, custom arrays may be more appropriate for some research questions. A critical component of microarrays is their dependence, and therefore potential limitation, on the identity of the genes on the array. Many commercially available arrays often do not have a large representation of genes expressed in a tissue as highly specialized as the retina. Low representation of genes from the retina could curtail the discovery of novel retina genes and hinder exploration of disease mechanisms. Secondly, the considerable cost of commercial arrays limits the number of experiments that can be performed. However, once established, custom arrays can be generated at a reasonable cost, increasing the number of replicates and time-points, and consequently improving the statistical analyses. Finally, recently published calibration experiments demonstrated that fold-change measurements using custom cDNA arrays were more predictable and accurate, and had less uncontrolled bias than oligonucleotide arrays {Yuen, 2002}.

There is a continuing need in the art to characterize degenerating or dying neuronal cells relative to normal neuronal cells so that any differences can be exploited for therapeutic and diagnostic benefits.

### SUMMARY OF THE INVENTION

A first embodiment of the invention provides a method for inhibiting neuronal cell death in a mammalian subject. An effective amount of an isolated molecule comprising an antibody variable region is administered to a subject in need thereof. The antibody variable region specifically binds to a neuronal marker (NM) protein selected from the group consisting of: Ceruloplasmin (ferroxidase); Adenylyl cyclase 6; Insulin-like growth factor 1 receptor; vascular endothelial growth factor; Rat mRNA for sucrase isomaltase (EC 3.2.1.10).; Serotonin (5-hydroxytryptamine (5HT)) receptor, type 1B; Fos like antigen 2; phospholipase C-III; Rat phospholipase C-III mRNA, complete cds.; interleukin 18; calcium channel, voltage-dependent, alpha2/delta subunit 1; Vesicle-associated membrane protein (synaptobrevin 2); putative; Rattus norvegicus G protein-coupled receptor (GPR19) gene, partial cds.; This sequence comes from FIG. 1b; A2 adenosine receptor [rats, striatum, mRNA, 2141 nt].; Max interacting protein 1; ATPase isoform 2, Na+K+ transporting, beta polypeptide 2; Secretory granule neuroendocrine, protein 1 (7B2 protein); Pim-1 oncogene; adenylate kinase 3; alpha-methylacyl-CoA racemase; Inhibitor of DNA binding 2, dominant negative helix-loop-helix protein; Rattus norvegicus mRNA for 20-alpha-hydroxysteroid dehydrogenase (20-alpha-HSD), complete cds; telomerase protein component 1; pyruvate dehydrogenase kinase, isoenzyme 1; Solute carrier family 4, member 2, anion exchange protein 2; phospholipase A2, group IIC; syntaxin 3; Rattus norvegicus mRNA for interleukin-4 receptor (soluble form), complete cds; Protein tyrosine phosphatase, non-receptor type substrate 1 (SHP substrate 1); B-cell translocation gene 2, anti-proliferative; Acyl-Coa dehydrogenase, Very long chain; Clusterin; syntaxin 4; Natriuretic peptide receptor A/Guanylate cyclase A; megakaryocyte-associated tyrosine kinase; presenilin-2; phospholipase A2, group VI; pancreatic lipase-related protein 2; phospholipase C, beta 3; Phospholipase C, gamma 1; Ephrin B1; Retinoblastoma-related gene; protein kinase C epsilon subspecies; Rat protein kinase C epsilon subspecies.; Spinocerebellar ataxia type 1; phospholipase A2, group V; Angiotensin I-converting enzyme (Dipeptidyl carboxypeptidase 1); Steroid sulfatase; protein kinase C zeta subspecies; Rat protein kinase C zeta subspecies.; Calcium channel alpha 1A; carcinoembryonic antigen-related cell adhesion molecule; amphiphysin; Rat glutathione S-transferase mRNA, complete cds; Cathepsin L; Acyl Coenzyme A dehydrogenase, long chain; ATP-binding cassette, sub-family B (MDR/TAP), member 1 (P-glycoprotein/multidrug resistance 1); c-fos protein (AA 1-380); Rat c-fos mRNA.; glutamate receptor, ionotropic, AMPA2 (alpha 2); syntaxin 6; dipeptidylpeptidase 6; G protein-coupled receptor kinase 2, groucho gene related (Drosophila); Max; protein kinase C alpha (AA 1-672); Rat mRNA for protein kinase C alpha.; fatty acid amide hydrolase; Carnitine palmitoyltransferase 1 alpha, liver isoform; calcium channel, voltage-dependent, L type, alpha 1D subunit; BRbeta B-regulatory subunit of protein phosphatase 2A; Secretogranin II; transmembrane receptor Unc5H2; potassium inwardly-rectifying channel, subfamily J, member 12; Acetylcholine receptor beta; B-cell translocation gene 1, anti-proliferative; Lectin, galactose binding, soluble 9 (Galectin-9); Insulin receptor; synaptotagmin 5; Rattus norvegicus calcium/calmodulin-dependent protein kinase II delta subunit mRNA, partial cds.; High mobility group 1; thyroid hormone receptor alpha; Rattus norvegicus cytochrome P450 4F5 (CYP4F5) mRNA, complete cds; Insulin-like growth factor 2 receptor; Rat glucagon receptor mRNA, complete cds; Arrestin, beta 1; protease (prosome, macropain) 26S subunit, ATPase 1; R. rattus mRNA for NPY-1 receptor.; kinase domain is 450..1295; Rattus rattus mRNA for PCTAIRE3, complete cds.; R. rattus RL/IF-1 mRNA.; Arrestin, beta 2; vascular endothelial growth factor; Ras-related small GTP binding protein 3A; Adenylyl cyclase 6; LIM motif-containing protein kinase 2; This sequence comes from FIG. 1b; A2 adenosine receptor [rats, striatum, mRNA, 2141 nt].; Adrenergic receptor kinase, beta 2 (G-protein-linked receptor kinase); Arrestin, beta 1; endothelial differentiation, sphingolipid G-protein-coupled receptor, 5; immediate early gene transcription factor NGFI-B; potassium inwardly-rectifying channel, subfamily J, member 12; Rattus norvegicus calcium/calmodulin-dependent protein kinase II delta subunit mRNA, partial cds.; interleukin 18; Max interacting protein 1; prostaglandin F2 receptor negative regulator; BRbeta B-regulatory subunit of protein phosphatase 2A; Protein tyrosine phosphatase, non-receptor type substrate 1 (SHP substrate 1); Rattus norvegicus insulin-regulated membrane aminopeptidase IRAP mRNA, complete cds; Ceruloplasmin (ferroxidase); cyclin-dependent kinase 5; adrenergic receptor kinase, beta 1; MAD (mothers against decapentaplegic, Drosophila) homolog 1; CamK I; calcium/calmodulin-dependent protein kinase type I +CaM-like protein kinase; Calcium channel alpha 1A; phosphofructokinase, muscle; p32-subunit of replication protein A; Rattus norvegicus mRNA for Janus protein tyrosine kinase 1, JAK1.; Rat insulin-like growth factor binding protein (rIGFBP-6) mRNA, complete cds.; Discoidin domain receptor (neurotrophic tyrosine kinase, receptor, type 4 (cell adhesion kinase)); Insulin-like growth factor 1 receptor; Tumor protein p53 (Li-Fraumeni syndrome); phospholipase A2, group VI; solute carrier family 2 (facilitated glucose transporter), member 5; Inhibitor of DNA binding 2, dominant negative helix-loop-helix protein; Rat mRNA for proteasome activator rPA28 subunit alpha, complete cds.; Protein tyrosine phosphatase, receptor type, A; aminopeptidase B; Rat mRNA for cyclin D1, complete cds.; syntaxin 5a; Natriuretic peptide receptor A/Guanylate cyclase A; TR4 orphan receptor; galanin receptor 2; casein kinase II, alpha 1 polypeptide; carcinoembryonic antigen-related cell adhesion molecule; protein tyrosine phosphatase, receptor type, R; Neurofibromatosis type 1; Rat glutathione S-transferase mRNA, complete cds; calcium channel, voltage-dependent, L type, alpha 1D subunit; Acetylcholine receptor alpha 3 (neuronal nicotine); mitogen activated protein kinase 3; mismatch repair protein; tissue inhibitor of metalloproteinase 2; Solute carrier family 4, member 2, anion exchange protein 2; Rat mRNA for multicatalytic proteionase (MCP) subunit L ingensin, Atp-dependent proteinase, proteasome, macropain).; Janus kinase 2 (a protein tyrosine kinase); kinase domain is 450..1295; Rattus rattus mRNA for PCTAIRE3, complete cds.; This sequence comes from FIG. 1; Na+/Cl(−)-dependent neurotransmitter transporter [rats, brain, mRNA, 3762 nt].; Set beta isoform; leukemogenesis protein; This sequence comes from FIG. 1 IIB; set=Set beta isoform {alternatively spliced} [rats, neonatal kidney, mRNA, 2026 nt].; synapsin II; Calmodulin III; subunit 8; R. rattus mRNA for glutathione transferase subunit 8.; Rattus norvegicus neuron-specific enolase (NSE) mRNA, complete cds; syntaxin 3; Tyrosine 3-monooxygenase/tryptophan 5-monooxygenase activation protein, eta polypeptide; Carnitine palmitoyltransferase 1 alpha, liver isoform; Superoxide dismutase 1, soluble; phospholipase C, beta 3; Angiotensin I-converting enzyme (Dipeptidyl carboxypeptidase 1); c-fos protein (AA 1-380); Rat c-fos mRNA.; transmembrane receptor Unc5H2; GTPase Rab14; ATP-binding cassette, sub-family C (CFTR/MRP), member 1 (multiple drug resistance-associated protein); Inhibitor of DNA binding 1, helix-loop-helix protein (splice variation); and dipeptidylpeptidase 6. Neuronal cell death is thereby inhibited.

A second embodiment of the invention provides a method for preventing neuronal cell death in a mammal. A nucleic acid molecule comprising a coding sequence for a NM protein is administed to the mammal. The coding sequence is selected from the group consisting of: NM Acetylcholine receptor alpha 5; Nerve growth factor receptor, fast; Rat insulin-like growth factor binding protein (rIGFBP-6) mRNA, complete cds.; transforming growth factor, beta receptor I; taurine/beta-alanine transporter; Rat mRNA for proteasome subunit RC10-II, complete cds.; C holinergic receptor, nicotinic, alpha polypeptide 7 (neuronal nicotinic acetycholine receptor alpha 7) (bungarotoxin alpha); 6-phosphofructo-2-kinase/fructose-2,6-biphosphatase 4; heterogeneous nuclear ribonucleoproteins methyltransferase-like 2 (S. cerevisiae); R. rattus mRNA for epididymal secretory glutathione peroxidase.; matrix metalloproteinase 14, membrane-inserted; cAMP response element binding protein; Solute carrier family 2 A3 (neuron glucose transporter); ATPase, Na+K+ transporting, alpha 1 polypeptide; Fyn proto-oncogene; protein kinase inhibitor, alpha; Rattus norvegicus galactosyltransferase associated kinase (GTA) mRNA, complete cds; Early growth response 1; Glutathione-S-transferase, placental enzyme pi type; neogenin; ATP synthase, H+ transporting, mitochondrial F0 complex, subunit c (subunit 9), isoform 1; 36 kDa calcium-dependent phospholipid-binding protein; This sequence comes from FIG. 1; conceptual translation differs that in published reference; calpactin 1; annexin II=36 kDa calcium-dependent phospholipid-binding protein [rats, RBL-2H3 basophilic leukemia cells, mRNA, 1362 nt].; Murine leukemia viral (v-raf-1) oncogene homolog 1 (3611-MSV); Inhibitor of DNA binding 1, helix-loop-helix protein (splice variation); alternative splicing: see also D28754; Rat mRNA for cyclin dependent kinase 2-alpha.; Tyrosine 3monooxygenase/tryptophan 5-monooxygenase activation protein, zeta polypeptide; Solute carrier family 25, member 5 (adenine nucleotid translocator 2, fibroblast isoform (ATP-ADP carrier protein)); Dopa decarboxylase (aromatic L-amino acid decarboxylase); cadherin 22; Rat thymidine kinase mRNA, 5′ end.; Solute carrier family18 (vesicular monoamine) member 1 (chromaffin granule amine transporter); mitogen-activated protein kinase 6; R. norvegicus mRNA for Cdk-activating kinase; ADP-ribosylatiori factor 2; mismatch repair protein; CD24 antigen; glutamate-cysteine ligase, modifier subunit; PDZ and LIM domain 1 (elfin); casein kinase II beta subunit; Inhibitor of DNA binding 3, dominant negative helix-loop-helix protein; Rattus norvegicus Sprague-Dawley lipid-binding protein mRNA, complete cds; Rat mRNA for cyclin D1, complete cds.; Proliferating cell nuclear antigen; bone morphogenetic protein 2; VGF nerve growth factor inducible; activity regulated cytoskeletal-associated protein; Fos-like antigen 1; Cyclin G1; taurine/beta-alanine transporter; Vesicle-associated membrane protein (synaptobrevin 2); unction plakoglobin; Inhibitor of DNA binding 3, dominant negative helix-loop-helix protein; Heat shock 27 kDa protein; Solute carrier family18 (vesicular monoamine) member 1 (chromaffin granule amine transporter); mitogen-activated protein kinase 6; Interleukin 6 signal transducer; Synaptophysin; latexin; Nerve growth factor receptor, fast; 36 kDa calcium-dependent phospholipid-binding protein; This sequence comes from FIG. 1; conceptual translation differs that in published reference; calpactin 1; annexin II=36 kDa calcium-dependent phospholipid-binding protein [rats, RBL-2H3 basophilic leukemia cells, mRNA, 1362 nt].; transcription factor AP-1 (AA 1-334); Rat c-jun oncogene mRNA for transcription factor AP-1.; B-cell translocation gene 1, anti-proliferativeputative anti-proliferative factor; glycoprotein hormones, alpha subunit; Adenomatosis polyposis coli; Rattus norvegicus jun-D gene, complete cds; R. rattus mRNA for heat shock protein 70.; solute carrier family 30 (zinc transporter), member 1 zinc transporter; Cathepsin L; eukaryotic initiation factor 5 (eIF-5); 3-hydroxy-3methylglutaryl-Coenzyme A synthase 1; cysteine-rich protein 3; Solute carrier family 7 member A1 (amino acid transporter cationic 1); Cytochrom P450 Lanosterol 14 alpha-demethylase; myc box dependent interacting protein 1; plectin; ATPase, Ca++ transporting, plasma membrane 1; Rattus norvegicus Sprague-Dawley lipid-binding protein mRNA, complete cds; cyclin-dependent kinase inhibitor 1A (P21); Annexin V; bone morphogenetic protein 2; 6-phosphofructo-2-kinase/fructose-2,6-biphosphatase 4; Tumor necrosis factor receptor superfamily, member 1a; ezrin; Pim-1 oncogene; Fos like antigen 2transcription factor; B-cell translocation gene 2, anti-proliferative; Rattus norvegicus RIN1 mRNA, complete cds; Rat brain glucose-transporter protein mRNA, complete cds; jun B proto-oncogene; VGF nerve growth factor inducible; Interleukin 2 receptor, beta chain; Early growth response 1; Rat mRNA for LDL-receptor; Rat mRNA for 53 kD polypeptide induced by growth factors (EGF) and oncogenes (H-ras; src; polyoma middle T); urinary plasminogen activator receptor 2urinary-type plasminogen activator receptor; Rat transformation-associated protein (34A) mRNA, complete cds; serine (or cysteine) proteinase inhibitor, clade E (nexin, plasminogen activator inhibitor type 1), member 1; Fos-like antigen 1; and activity regulated cytoskeletal-associated protein. Neuronal cell death in the mammal is thereby inhibited or prevented.

A third embodiment of the invention is a method for preventing neuronal cell death in a mammal. A purified human NM protein selected from the group consisting of: NM Acetylcholine receptor alpha 5; Nerve growth factor receptor, fast; Rat insulin-like growth factor binding protein (rIGFBP-6) mRNA, complete cds.; transforming growth factor, beta receptor I; taurine/beta-alanine transporter; Rat mRNA for proteasome subunit RC10-II, complete cds.; C holinergic receptor, nicotinic, alpha polypeptide 7 (neuronal nicotinic acetycholine receptor alpha 7) (bungarotoxin alpha); 6-phosphofructo-2-kinase/fructose-2,6-biphosphatase 4; heterogeneous nuclear ribonucleoproteins methyltransferase-like 2 (S. cerevisiae); R. rattus mRNA for epididymal secretory glutathione peroxidase.; matrix metalloproteinase 14, membrane-inserted; cAMP response element binding protein; Solute carrier family 2 A3 (neuron glucose transporter); ATPase, Na+K+ transporting, alpha 1 polypeptide; Fyn proto-oncogene; protein kinase inhibitor, alpha; Rattus norvegicus galactosyltransferase associated kinase (GTA) mRNA, complete cds; Early growth response 1; Glutathione-S-transferase, placental enzyme pi type; neogenin; ATP synthase, H+ transporting, mitochondrial F0 complex, subunit c (subunit 9), isoform 1; 36 kDa calcium-dependent phospholipid-binding protein; This sequence comes from FIG. 1; conceptual translation differs that in published reference; calpactin 1; annexin II=36 kDa calcium-dependent phospholipid-binding protein [rats, RBL-2H3 basophilic leukemia cells, mRNA, 1362 nt].; Murine leukemia viral (v-raf-1) oncogene homolog 1 (3611-MSV); Inhibitor of DNA binding 1, helix-loop-helix protein (splice variation); alternative splicing: see also D28754; Rat mRNA for cyclin dependent kinase 2-alpha.; Tyrosine 3-monooxygenase/tryptophan 5monooxygenase activation protein, zeta polypeptide; Solute carrier family 25, member 5 (adenine nucleotid translocator 2, fibroblast isoform (ATP-ADP carrier protein)); Dopa decarboxylase (aromatic L-amino acid decarboxylase); cadherin 22; Rat thymidine kinase mRNA, 5′ end.; Solute carrier family18 (vesicular monoamine) member 1 (chromaffin granule amine transporter); mitogen-activated protein kinase 6; R. norvegicus mRNA for Cdk-activating kinase; ADP-ribosylation factor 2; mismatch repair protein; CD24 antigen; glutamate-cysteine ligase, modifier subunit; PDZ and LIM domain 1 (elfin); casein kinase II beta subunit; Inhibitor of DNA binding 3, dominant negative helix-loop-helix protein; Rattus norvegicus Sprague-Dawley lipid-binding protein mRNA, complete cds; Rat mRNA for cyclin D1, complete cds.; Proliferating cell nuclear antigen; bone morphogenetic protein 2; VGF nerve growth factor inducible; activity regulated cytoskeletal-associated protein; Fos-like antigen 1; Cyclin G1; taurine/beta-alanine transporter; Vesicle-associated membrane protein (synaptobrevin 2); unction plakoglobin; Inhibitor of DNA binding 3, dominant negative helix-loop-helix protein; Heat shock 27 kDa protein; Solute carrier family18 (vesicular monoamine) member 1 (chromaffin granule amine transporter); mitogen-activated protein kinase 6; Interleukin 6 signal transducer; Synaptophysin; latexin; Nerve growth factor receptor, fast; 36 kDa calcium-dependent phospholipid-binding protein; This sequence comes from FIG. 1; conceptual translation differs that in published reference; calpactin 1; annexin II=36 kDa calcium-dependent phospholipid-binding protein [rats, RBL-2H3 basophilic leukemia cells, mRNA, 1362 nt].; transcription factor AP-1 (AA 1-334); Rat c-jun oncogene mRNA for transcription factor AP-1.; B-cell translocation gene 1, anti-proliferativeputative anti-proliferative factor; glycoprotein hormones, alpha subunit; Adenomatosis polyposis coli; Rattus norvegicus jun-D gene, complete cds; R. rattus mRNA for heat shock protein 70.; solute carrier family 30 (zinc transporter), member 1zinc transporter; Cathepsiri L; eukaryotic initiation factor 5 (eIF-5); 3-hydroxy-3-methylglutaryl-Coenzyme A synthase 1; cysteine-rich protein 3; Solute carrier family 7 member A1 (amino acid transporter cationic 1); Cytochrom P450 Lanosterol 14 alpha-demethylase; myc box dependent interacting protein 1; plectin; ATPase, Ca++ transporting, plasma membrane 1; Rattus norvegicus Sprague-Dawley lipid-binding protein mRNA, complete cds; cyclin-dependent kinase inhibitor 1A (P21); Annexin V; bone morphogenetic protein 2; 6phosphofructo-2-kinase/fructose-2,6-biphosphatase 4; Tumor necrosis factor receptor superfamily, member 1a; ezrin; Pim-1 oncogene; Fos like antigen 2transcription factor; B-cell translocation gene 2, anti-proliferative; Rattus norvegicus RIN1 mRNA, complete cds; Rat brain glucose-transporter protein mRNA, complete cds; jun B proto-oncogene; VGF nerve growth factor inducible; Interleukin 2 receptor, beta chain; Early growth response 1; Rat mRNA for LDL-receptor; Rat mRNA for 53 kD polypeptide induced by growth factors (EGF) and oncogenes (H-ras; src; polyoma middle T); urinary plasminogen activator receptor 2urinary-type plasminogen activator receptor; Rat transformation-associated protein (34A) mRNA, complete cds; serine (or cysteine) proteinase inhibitor, clade E (nexin, plasminogen activator inhibitor type 1), member 1; Fos-like antigen 1; and activity regulated cytoskeletal-associated protein is administered to the mammal. Neuronal cell death in the mammal is thereby inhibited or prevented.

A fourth embodiment of the invention is a method of identifying regions of neuronal cell death in a patient. A molecule comprising an antibody variable region is administered to the patient. The molecule is bound to a detectable moiety. The antibody variable region specifically binds to a NM protein selected from the group consisting of: Ceruloplasmin (ferroxidase); Adenylyl cyclase 6; Insulin-like growth factor 1 receptor; vascular endothelial growth factor; Rat mRNA for sucrase isomaltase (EC 3.2.1.10).; Serotonin (5hydroxytryptamine (5HT)) receptor, type 1B; Fos like antigen 2; phospholipase C-111; Rat phospholipase C-III mRNA, complete cds.; interleukin 18; calcium channel, voltage-dependent, alpha2/delta subunit 1; Vesicle-associated membrane protein (synaptobrevin 2); putative; Rattus norvegicus G protein-coupled receptor (GPR19) gene, partial cds.; This sequence comes from FIG. 1b; A2 adenosine receptor [rats, striatum, mRNA, 2141 nt].; Max interacting protein 1; ATPase isoform 2, Na+K+ transporting, beta polypeptide 2; Secretory granule neuroendocrine, protein 1 (7B2 protein); Pim-1 oncogene; adenylate kinase 3; alpha-methylacyl-CoA racemase; Inhibitor of DNA binding 2, dominant negative helix-loop-helix protein; Rattus norvegicus mRNA for 20-alpha-hydroxysteroid dehydrogenase (20-alpha-HSD), complete cds; telomerase protein component 1; pyruvate dehydrogenase kinase, isoenzyme 1; Solute carrier family 4, member 2, anion exchange protein 2; phospholipase A2, group IIC; syntaxin 3; Rattus norvegicus mRNA for interleukin-4 receptor (soluble form), complete cds; Protein tyrosine phosphatase, non-receptor type substrate 1 (SHP substrate 1); B-cell translocation gene 2, anti-proliferative; Acyl-Coa dehydrogenase, Very long chain; Clusterin; syntaxin 4; Natriuretic peptide receptor A/Guanylate cyclase A; megakaryocyte-associated tyrosine kinase; presenilin-2; phospholipase A2, group VI; pancreatic lipase-related protein 2; phospholipase C, beta 3; Phospholipase C, gamma 1; Ephrin B1; Retinoblastoma-related gene; protein kinase C epsilon subspecies; Rat protein kinase C epsilon subspecies.; Spinocerebellar ataxia type 1; phospholipase A2, group V; Angiotensin I-converting enzyme (Dipeptidyl carboxypeptidase 1); Steroid sulfatase; protein kinase C zeta subspecies; Rat protein kinase C zeta subspecies.; Calcium channel alpha 1A; carcinoembryonic antigen-related cell adhesion molecule; amphiphysin; Rat glutathione S-transferase mRNA, complete cds; Cathepsin L; Acyl Coenzyme A dehydrogenase, long chain; ATP-binding cassette, sub-family B (MDR/TAP), member 1 (P-glycoprotein/multidrug resistance 1); c-fos protein (AA 1-380); Rat c-fos mRNA.; glutamate receptor, ionotropic, AMPA2 (alpha 2); syntaxin 6; dipeptidylpeptidase 6; G protein-coupled receptor kinase 2, groucho gene related (Drosophila); Max; protein kinase C alpha (AA 1-672); Rat mRNA for protein kinase C alpha.; fatty acid amide hydrolase; Carnitine palmitoyltransferase 1 alpha, liver isoform; calcium channel, voltage-dependent, L type, alpha 1D subunit; BRbeta B-regulatory subunit of protein phosphatase 2A; Secretogranin II; transmembrane receptor Unc5H2; potassium inwardly-rectifying channel, subfamily J, member 12; Acetylcholine receptor beta; B-cell translocation gene 1, anti-proliferative; Lectin, galactose binding, soluble 9 (Galectin-9); Insulin receptor; synaptotagmin 5; Rattus norvegicus calcium/calmodulin-dependent protein kinase II delta subunit mRNA, partial cds.; High mobility group 1; thyroid hormone receptor alpha; Rattus norvegicus cytochrome P450 4F5 (CYP4F5) mRNA, complete cds; Insulin-like growth factor 2 receptor; Rat glucagon receptor mRNA, complete cds; Arrestin, beta 1; protease (prosome, macropain) 26S subunit, ATPase 1; R. rattus mRNA for NPY-1 receptor.; kinase domain is 450..1295; Rattus rattus mRNA for PCTAIRE3, complete cds.; R. rattus RL/IF-1 mRNA.; Arrestin, beta 2; vascular endothelial growth factor; Ras-related small GTP binding protein 3A; Adenylyl cyclase 6; LIM motif-containing protein kinase 2; This sequence comes from FIG. 1b; A2 adenosine receptor [rats, striatum, mRNA, 2141 nt].; Adrenergic receptor kinase, beta 2 (G-protein-linked receptor kinase); Arrestin, beta 1; endothelial differentiation, sphingolipid G-protein-coupled receptor, 5; immediate early gene transcription factor NGFI-B; potassium inwardly-rectifying channel, subfamily J, member 12; Rattus norvegicus calcium/calmodulin-dependent protein kinase II delta subunit mRNA, partial cds.; interleukin 18; Max interacting protein 1; prostaglandin F2 receptor negative regulator; BRbeta B-regulatory subunit of protein phosphatase 2A; Protein tyrosine phosphatase, non-receptor type substrate 1 (SHP substrate 1); Rattus norvegicus insulin-regulated membrane aminopeptidase IRAP mRNA, complete cds; Ceruloplasmin (ferroxidase); cyclin-dependent kinase 5; adrenergic receptor kinase, beta 1; MAD (mothers against decapentaplegic, Drosophila) homolog 1; CamK I; calcium/calmodulin-dependent protein kinase type I +CaM-like protein kinase; Calcium channel alpha 1A; phosphofructokinase, muscle; p32-subunit of replication protein A; Rattus norvegicus mRNA for Janus protein tyrosine kinase 1, JAK1.; Rat insulin-like growth factor binding protein (rIGFBP-6) mRNA, complete cds.; Discoidin domain receptor (neurotrophic tyrosine kinase, receptor, type 4 (cell adhesion kinase)); Insulin-like growth factor 1 receptor; Tumor protein p53 (Li-Fraumeni syndrome); phospholipase A2, group VI; solute carrier family 2 (facilitated glucose transporter), member 5; Inhibitor of DNA binding 2, dominant negative helix-loop-helix protein; Rat mRNA for proteasome activator rPA28 subunit alpha, complete cds.; Protein tyrosine phosphatase, receptor type, A; aminopeptidase B; Rat mRNA for cyclin D1, complete cds.; syntaxin 5a; Natriuretic peptide receptor A/Guanylate cyclase A; TR4 orphan receptor; galanin receptor 2; casein kinase II, alpha 1 polypeptide; carcinoembryonic antigen-related cell adhesion molecule; protein tyrosine phosphatase, receptor type, R; Neurofibromatosis type 1; Rat glutathione S-transferase mRNA, complete cds; calcium channel, voltage-dependent, L type, alpha 1D subunit; Acetylcholine receptor alpha 3 (neuronal nicotine); mitogen activated protein kinase 3; mismatch repair protein; tissue inhibitor of metalloproteinase 2; Solute carrier family 4, member 2, anion exchange protein 2; Rat mRNA for multicatalytic proteionase (MCP) subunit L ingensin, Atp-dependent proteinase, proteasome, macropain).; Janus kinase 2 (a protein tyrosine kinase); kinase domain is 450..1295; Rattus rattus mRNA for PCTAIRE3, complete cds.; This sequence comes from FIG. 1; Na+/Cl(−)-dependent neurotransmitter transporter [rats, brain, mRNA, 3762 nt].; Set beta isoform; leukemogenesis protein; This sequence comes from FIG. 1 IIB; set=Set beta isoform {alternatively spliced} [rats, neonatal kidney, mRNA, 2026 nt].; synapsin II; Calmodulin III; subunit 8; R. rattus mRNA for glutathione transferase subunit 8.; Rattus norvegicus neuron-specific enolase (NSE) mRNA, complete cds; syntaxin 3; Tyrosine 3-monooxygenase/tryptophan 5-monooxygenase activation protein, eta polypeptide; Carnitine palmitoyltransferase 1-alpha, liver isoform; Superoxide dismutase 1, soluble; phospholipase C, beta 3; Angiotensin I-converting enzyme (Dipeptidyl carboxypeptidase 1); c-fos protein (AA 1-380); Rat c-fos mRNA.; transmembrane receptor Unc5H2; GTPase Rab14; ATP-binding cassette, sub-family C (CFTR/MRP), member 1 (multiple drug resistance-associated protein); Inhibitor of DNA binding 1, helix-loop-helix protein (splice variation); and dipeptidylpeptidase 6. The detectable moiety in the pateint is detected. Regions of neuronal cell death are thereby detected.

A fifth embodiment of the invention is a method of screening for neuronal cell death in a patient. A body fluid collected from the patient is contacted with a molecule comprising an antibody variable region which specifically binds to a NM protein selected from the group consisting of: Ceruloplasmin (ferroxidase); Adenylyl cyclase 6; Insulin-like growth factor 1 receptor; vascular endothelial growth factor; Rat mRNA for sucrase isomaltase (EC 3.2.1.10).; Serotonin (5-hydroxytryptamine (5HT)) receptor, type 1B; Fos like antigen 2; phospholipase C-11; Rat phospholipase C-III mRNA, complete cds.; interleukin 18; calcium channel, voltage-dependent, alpha2/delta subunit 1; Vesicle-associated membrane protein (synaptobrevin 2); putative; Rattus norvegicus G protein-coupled receptor (GPR19) gene, partial cds.; This sequence comes from FIG. 1b; A2 adenosine receptor [rats, striatum, mRNA, 2141 nt].; Max interacting protein 1; ATPase isoform 2, Na+K+ transporting, beta polypeptide 2; Secretory granule neuroendocrine, protein 1 (7B2 protein); Pim-1 oncogene; adenylate kinase 3; alpha-methylacyl-CoA racemase; Inhibitor of DNA binding 2, dominant negative helix-loop-helix protein; Rattus norvegicus mRNA for 20-alpha-hydroxysteroid dehydrogenase (20-alpha-HSD), complete cds; telomerase protein component 1; pyruvate dehydrogenase kinase, isoenzyme 1; Solute carrier family 4, member 2, anion exchange protein 2; phospholipase A2, group IIC; syntaxin 3; Rattus norvegicus mRNA for interleukin-4 receptor (soluble form), complete cds; Protein tyrosine phosphatase, non-receptor type substrate 1 (SHP substrate 1); B-cell translocation gene 2, anti-proliferative; Acyl-Coa dehydrogenase, Very long chain; Clusterin; syntaxin 4; Natriuretic peptide receptor A/Guanylate cyclase A; megakaryocyte-associated tyrosine kinase; presenilin-2; phospholipase A2, group VI; pancreatic lipase-related protein 2; phospholipase C, beta 3; Phospholipase C, gamma 1; Ephrin B1; Retinoblastoma-related gene; protein kinase C epsilon subspecies; Rat protein kinase C epsilon subspecies.; Spinocerebellar ataxia type 1; phospholipase A2, group V; Angiotensin I-converting enzyme (Dipeptidyl carboxypeptidase 1); Steroid sulfatase; protein kinase C zeta subspecies; Rat protein kinase C zeta subspecies.; Calcium channel alpha 1A; carcinoembryonic antigen-related cell adhesion molecule; amphiphysin; Rat glutathione S-transferase mRNA, complete cds; Cathepsin L; Acyl Coenzyme A dehydrogenase, long chain; ATP-binding cassette, sub-family B (MDR/TAP), member 1 (P-glycoprotein/multidrug resistance 1); c-fos protein (AA 1-380); Rat c-fos mRNA.; glutamate receptor, ionotropic, AMPA2 (alpha 2); syntaxin 6; dipeptidylpeptidase 6; G protein-coupled receptor kinase 2, groucho gene related (Drosophila); Max; protein kinase C alpha (AA 1-672); Rat mRNA for protein kinase C alpha.; fatty acid amide hydrolase; Carnitine palmitoyltransferase 1 alpha, liver isoform; calcium channel, voltage-dependent, L type, alpha 1D subunit; BRbeta B-regulatory subunit of protein phosphatase 2A; Secretogranin II; transmembrane receptor Unc5H2; potassium inwardly-rectifying channel, subfamily J, member 12; Acetylcholine receptor beta; B-cell translocation gene 1, anti-proliferative; Lectin, galactose binding, soluble 9 (Galectin-9); Insulin receptor; synaptotagmin 5; Rattus norvegicus calcium/calmodulin-dependent protein kinase II delta subunit mRNA, partial cds.; High mobility group 1; thyroid hormone receptor alpha; Rattus norvegicus cytochrome P450 4F5 (CYP4F5) mRNA, complete cds; Insulin-like growth factor 2 receptor; Rat glucagon receptor mRNA, complete cds; Arrestin, beta 1; protease (prosome, macropain) 26S subunit, ATPase 1; R. rattus mRNA for NPY-1 receptor.; kinase domain is 450..1295; Rattus rattus mRNA for PCTAIRE3, complete cds.; R. rattus RL/IF-1 mRNA.; Arrestin, beta 2; vascular endothelial growth factor; Ras-related small GTP binding protein 3A; Adenylyl cyclase 6; LIM motif-containing protein kinase 2; This sequence comes from FIG. 1b; A2 adenosine receptor [rats, striatum, mRNA, 2141 nt].; Adrenergic receptor kinase, beta 2 (G-protein-linked receptor kinase); Arrestin, beta 1; endothelial differentiation, sphingolipid G-protein-coupled receptor, 5; immediate early gene transcription factor NGFI-B; potassium inwardly-rectifying channel, subfamily J, member 12; Rattus norvegicus calcium/calmodulin-dependent protein kinase II delta subunit mRNA, partial cds.; interleukin 18; Max interacting protein 1; prostaglandin F2 receptor negative regulator; BRbeta B-regulatory subunit of protein phosphatase 2A; Protein tyrosine phosphatase, non-receptor type substrate 1 (SHP substrate 1); Rattus norvegicus insulin-regulated membrane aminopeptidase IRAP mRNA, complete cds; Ceruloplasmin (ferroxidase); cyclin-dependent kinase 5; adrenergic receptor kinase, beta 1; MAD (mothers against decapentaplegic, Drosophila) homolog 1; CamK I; calcium/calmodulin-dependent protein kinase type I +CaM-like protein kinase; Calcium channel alpha 1A; phosphofructokinase, muscle; p32-subunit of replication protein A; Rattus norvegicus mRNA for Janus protein tyrosine kinase 1, JAK1.; Rat insulin-like growth factor binding protein (rIGFBP-6) mRNA, complete cds.; Discoidin domain receptor (neurotrophic tyrosine kinase, receptor, type 4 (cell adhesion kinase)); Insulin-like growth factor 1 receptor; Tumor protein p53 (Li-Fraumeni syndrome); phospholipase A2, group VI; solute carrier family 2 (facilitated glucose transporter), member 5; Inhibitor of DNA binding 2, dominant negative helix-loop-helix protein; Rat mRNA for proteasome activator rPA28 subunit alpha, complete cds.; Protein tyrosine phosphatase, receptor type, A; aminopeptidase B; Rat mRNA for cyclin D1, complete cds.; syntaxin 5a; Natriuretic peptide receptor A/Guanylate cyclase A; TR4 orphan receptor; galanin receptor 2; casein kinase II, alpha 1 polypeptide; carcinoembryonic antigen-related cell adhesion molecule; protein tyrosine phosphatase, receptor type, R; Neurofibromatosis type 1; Rat glutathione S-transferase mRNA, complete cds; calcium channel, voltage-dependent, L type, alpha 1D subunit; Acetylcholine receptor alpha 3 (neuronal nicotine); mitogen activated protein kinase 3; mismatch repair protein; tissue inhibitor of metalloproteinase 2; Solute carrier family 4, member 2, anion exchange protein 2; Rat mRNA for multicatalytic proteionase (MCP) subunit L ingensin, Atp-dependent proteinase, proteasome, macropain).; Janus kinase 2 (a protein tyrosine kinase); kinase domain is 450..1295; Rattus rattus mRNA for PCTAIRE3, complete cds.; This sequence comes from FIG. 1; Na+/Cl(−)-dependent neurotransmitter transporter [rats, brain, mRNA, 3762 nt].; Set beta isoform; leukemogenesis protein; This sequence comes from FIG. 1 IIB; set=Set beta isoform {alternatively spliced} [rats, neonatal kidney, mRNA, 2026 nt].; synapsin II; Calmodulin III; subunit 8; R. rattus mRNA for glutathione transferase subunit 8.; Rattus norvegicus neuron-specific enolase (NSE) mRNA, complete cds; syntaxin 3; Tyrosine 3-monooxygenase/tryptophan 5-monooxygenase activation protein, eta polypeptide; Carnitine palmitoyltransferase 1 alpha, liver isoform; Superoxide dismutase 1, soluble; phospholipase C, beta 3; Angiotensin I-converting enzyme (Dipeptidyl carboxypeptidase 1); c-fos protein (AA 1-380); Rat c-fos mRNA.; transmembrane receptor Unc5H2; GTPase Rab14; ATP-binding cassette, sub-family C (CFTR/MRP), member 1 (multiple drug resistance-associated protein); Inhibitor of DNA binding 1, helix-loop-helix protein (splice variation); and dipeptidylpeptidase 6. Detection of cross-reactive material in the body fluid with the molecule indicates neuronal cell death in the patient.

A sixth embodiment of the invention is method for promoting neuronal cell death in a patient. An NM protein selected from the group consising of: Ceruloplasmin (ferroxidase); Adenylyl cyclase 6; Insulin-like growth factor 1 receptor; vascular endothelial growth factor; Rat mRNA for sucrase isomaltase (EC 3.2.1.10).; Serotonin (5hydroxytryptamine (5HT)) receptor, type 1B; Fos like antigen 2; phospholipase C-111; Rat phospholipase C-III mRNA, complete cds.; interleukin 18; calcium channel, voltage-dependent, alpha2/delta subunit 1; Vesicle-associated membrane protein (synaptobrevin 2); putative; Rattus norvegicus G protein-coupled receptor (GPR19) gene, partial cds.; This sequence comes from FIG. 1b; A2 adenosine receptor [rats, striatum, mRNA, 2141 nt].; Max interacting protein 1; ATPase isoform 2, Na+K+ transporting, beta polypeptide 2; Secretory granule neuroendocrine, protein 1 (7B2 protein); Pim-1 oncogene; adenylate kinase 3; alpha-methylacyl-CoA racemase; Inhibitor of DNA binding 2, dominant negative helix-loop-helix protein; Rattus norvegicus mRNA for 20-alpha-hydroxysteroid dehydrogenase (20-alpha-HSD), complete cds; telomerase protein component 1; pyruvate dehydrogenase kinase, isoenzyme 1; Solute carrier family 4, member 2, anion exchange protein 2; phospholipase A2, group IIC; syntaxin 3; Rattus norvegicus mRNA for interleukin-4 receptor (soluble form), complete cds; Protein tyrosine phosphatase, non-receptor type substrate 1 (SHP substrate 1); B-cell translocation gene 2, anti-proliferative; Acyl-Coa dehydrogenase, Very long chain; Clusterin; syntaxin 4; Natriuretic peptide receptor A/Guanylate cyclase A; megakaryocyte-associated tyrosine kinase; presenilin-2; phospholipase A2, group VI; pancreatic lipase-related protein 2; phospholipase C, beta 3; Phospholipase C, gamma 1; Ephrin B1; Retinoblastoma-related gene; protein kinase C epsilon subspecies; Rat protein kinase C epsilon subspecies.; Spinocerebellar ataxia type 1; phospholipase A2, group V; Angiotensin I-converting enzyme (Dipeptidyl carboxypeptidase 1); Steroid sulfatase; protein kinase C zeta subspecies; Rat protein kinase C zeta subspecies.; Calcium channel alpha 1A; carcinoembryonic antigen-related cell adhesion molecule; amphiphysin; Rat glutathione S-transferase mRNA, complete cds; Cathepsin L; Acyl Coenzyme A dehydrogenase, long chain; ATP-binding cassette, sub-family B (MDR/TAP), member 1 (P-glycoprotein/multidrug resistance 1); c-fos protein (AA 1-380); Rat c-fos mRNA.; glutamate receptor, ionotropic, AMPA2 (alpha 2); syntaxin 6; dipeptidylpeptidase 6; G protein-coupled receptor kinase 2, groucho gene related (Drosophila); Max; protein kinase C alpha (AA 1-672); Rat mRNA for protein kinase C alpha.; fatty acid amide hydrolase; Carnitine palmitoyltransferase 1 alpha, liver isoform; calcium channel, voltage-dependent, L type, alpha 1D subunit; BRbeta B-regulatory subunit of protein phosphatase 2A; Secretogranin II; transmembrane receptor Unc5H2; potassium inwardly-rectifying channel, subfamily J, member 12; Acetylcholine receptor beta; B-cell translocation gene 1, anti-proliferative; Lectin, galactose binding, soluble 9 (Galectin-9); Insulin receptor; synaptotagmin 5; Rattus norvegicus calcium/calmodulin-dependent protein kinase II delta subunit mRNA, partial cds.; High mobility group 1; thyroid hormone receptor alpha; Rattus norvegicus cytochrome P450 4F5 (CYP4F5) mRNA, complete cds; Insulin-like growth factor 2 receptor; Rat glucagon receptor mRNA, complete cds; Arrestin, beta 1; protease (prosome, macropain) 26S subunit, ATPase 1; R. rattus mRNA for NPY-1 receptor.; kinase domain is 450..1295; Rattus rattus mRNA for PCTAIRE3, complete cds.; R. rattus RL/IF-1 mRNA.; Arrestin, beta 2; vascular endothelial growth factor; Ras-related small GTP binding protein 3A; Adenylyl cyclase 6; LIM motif-containing protein kinase 2; This sequence comes from FIG. 1b; A2 adenosine receptor [rats, striatum, mRNA, 2141 nt].; Adrenergic receptor kinase, beta 2 (G-protein-linked receptor kinase); Arrestin, beta 1; endothelial differentiation, sphingolipid G-protein-coupled receptor, 5; immediate early gene transcription factor NGFI-B; potassium inwardly-rectifying channel, subfamily J, member 12; Rattus norvegicus calcium/calmodulin-dependent protein kinase II delta subunit mRNA, partial cds.; interleukin 18; Max interacting protein 1; prostaglandin F2 receptor negative regulator; BRbeta B-regulatory subunit of protein phosphatase 2A; Protein tyrosine phosphatase, non-receptor type substrate 1 (SHP substrate 1); Rattus norvegicus insulin-regulated membrane aminopeptidase IRAP mRNA, complete cds; Ceruloplasmin (ferroxidase); cyclin-dependent kinase 5; adrenergic receptor kinase, beta 1; MAD (mothers against decapentaplegic, Drosophila) homolog 1; CamK I; calcium/calmodulin-dependent protein kinase type I +CaM-like protein kinase; Calcium channel alpha 1A; phosphofructokinase, muscle; p32-subunit of replication protein A; Rattus norvegicus mRNA for Janus protein tyrosine kinase 1, JAK1.; Rat insulin-like growth factor binding protein (rIGFBP-6) mRNA, complete cds.; Discoidin domain receptor (neurotrophic tyrosine kinase, receptor, type 4 (cell adhesion kinase)); Insulin-like growth factor 1 receptor; Tumor protein p53 (Li-Fraumeni syndrome); phospholipase A2, group VI; solute carrier family 2 (facilitated glucose transporter), member 5; Inhibitor of DNA binding 2, dominant negative helix-loop-helix protein; Rat mRNA for proteasome activator rPA28 subunit alpha, complete cds.; Protein tyrosine phosphatase, receptor type, A; aminopeptidase B; Rat mRNA for cyclin D1, complete cds.; syntaxin 5a; Natriuretic peptide receptor A/Guanylate cyclase A; TR4 orphan receptor; galanin receptor 2; casein kinase II, alpha 1 polypeptide; carcinoembryonic antigen-related cell adhesion molecule; protein tyrosine phosphatase, receptor type, R; Neurofibromatosis type 1; Rat glutathione S-transferase mRNA, complete cds; calcium channel, voltage-dependent, L type, alpha 1D subunit; Acetylcholine receptor alpha 3 (neuronal nicotine); mitogen activated protein kinase 3; mismatch repair protein; tissue inhibitor of metalloproteinase 2; Solute carrier family 4, member 2, anion exchange protein 2; Rat mRNA for multicatalytic proteionase (MCP) subunit L ingensin, Atp-dependent proteinase, proteasome, macropain).; Janus kinase 2 (a protein tyrosine kinase); kinase domain is 450..1295; Rattus rattus mRNA for PCTAIRE3, complete cds.; This sequence comes from FIG. 1; Na+/Cl(−)-dependent neurotransmitter transporter [rats, brain, mRNA, 3762 nt].; Set beta isoform; leukemogenesis protein; This sequence comes from FIG. 1 IIB; set=Set beta isoform {alternatively spliced} [rats, neonatal kidney, mRNA, 2026 nt].; synapsin II; Calmodulin III; subunit 8; R. rattus mRNA for glutathione transferase subunit 8.; Rattus norvegicus neuron-specific enolase (NSE) mRNA, complete cds; syntaxin 3; Tyrosine 3-monooxygenase/tryptophan 5monooxygenase activation protein, eta polypeptide; Carnitine palmitoyltransferase 1 alpha, liver isoform; Superoxide dismutase 1, soluble; phospholipase C, beta 3; Angiotensin I-converting enzyme (Dipeptidyl carboxypeptidase 1); c-fos protein (AA 1-380); Rat c-fos mRNA.; transmembrane receptor Unc5H2; GTPase Rab14; ATP-binding cassette, sub-family C (CFTR/MRP), member 1 (multiple drug resistance-associated protein); Inhibitor of DNA binding 1, helix-loop-helix protein (splice variation); and dipeptidylpeptidase 6 is administered to the patient. Neuronal cell death in the patient is thereby stimulated.

A seventh embodiment of the invention is a method of promoting neuronal cell death in a patient. A nucleic acid molecule encoding a NM protein is administered to the patient. The NM protein is selected from the group consising of Ceruloplasmin (ferroxidase); Adenylyl cyclase 6; Insulin-like growth factor 1 receptor; vascular endothelial growth factor; Rat mRNA for sucrase isomaltase (EC 3.2.1.10).; Serotonin (5-hydroxytryptamine (5HT)) receptor, type 1B; Fos like antigen 2; phospholipase C-III; Rat phosphblipase C-III mRNA, complete cds.; interleukin 18; calcium channel, voltage-dependent, alpha2/delta subunit 1; Vesicle-associated membrane protein (synaptobrevin 2); putative, Rattus norvegicus G protein-coupled receptor (GPR19) gene, partial cds.; This sequence comes from FIG. 1b; A2 adenosine receptor [rats, striatum, mRNA, 2141 nt].; Max interacting protein 1; ATPase isoform 2, Na+K+ transporting, beta polypeptide 2; Secretory granule neuroendocrine, protein 1 (7B2 protein); Pim-1 oncogene; adenylate kinase 3; alpha-methylacyl-CoA racemase; Inhibitor of DNA binding 2, dominant negative helix-loop-helix protein; Rattus norvegicus mRNA for 20-alpha-hydroxysteroid dehydrogenase (20-alpha-HSD), complete cds; telomerase protein component 1; pyruvate dehydrogenase kinase, isoenzyme 1; Solute carrier family 4, member 2, anion exchange protein 2; phospholipase A2, group IIC; syntaxin 3; Rattus norvegicus mRNA for interleukin-4 receptor (soluble form), complete cds; Protein tyrosine phosphatase, non-receptor type substrate 1 (SHP substrate 1); B-cell translocation gene 2, anti-proliferative; Acyl-Coa dehydrogenase, Very long chain; Clusterin; syntaxin 4; Natriuretic peptide receptor A/Guanylate cyclase A; megakaryocyte-associated tyrosine kinase; presenilin-2; phospholipase A2, group VI; pancreatic lipase-related protein 2; phospholipase C, beta 3; Phospholipase C, gamma 1; Ephrin B1; Retinoblastoma-related gene; protein kinase C epsilon subspecies; Rat protein kinase C epsilon subspecies.; Spinocerebellar ataxia type 1; phospholipase A2, group V; Angiotensin I-converting enzyme (Dipeptidyl carboxypeptidase 1); Steroid sulfatase; protein kinase C zeta subspecies; Rat protein kinase C zeta subspecies.; Calcium channel alpha 1A; carcinoembryonic antigen-related cell adhesion molecule; amphiphysin; Rat glutathione S-transferase mRNA, complete cds; Cathepsin L; Acyl Coenzyme A dehydrogenase, long chain; ATP-binding cassette, sub-family B (MDR/TAP), member 1 (P-glycoprotein/multidrug resistance 1); c-fos protein (AA 1-380); Rat c-fos mRNA.; glutamate receptor, ionotropic, AMPA2 (alpha 2); syntaxin 6; dipeptidylpeptidase 6; G protein-coupled receptor kinase 2, groucho gene related (Drosophila); Max; protein kinase C alpha (AA 1-672); Rat mRNA for protein kinase C alpha.; fatty acid amide hydrolase; Carnitine palmitoyltransferase 1 alpha, liver isoform; calcium channel, voltage-dependent, L type, alpha 1D subunit; BRbeta B-regulatory subunit of protein phosphatase 2A; Secretogranin II; transmembrane receptor Unc5H2; potassium inwardly-rectifying channel, subfamily J, member 12; Acetylcholine receptor beta; B-cell translocation gene 1, anti-proliferative; Lectin, galactose binding, soluble 9 (Galectin-9); Insulin receptor; synaptotagmin 5; Rattus norvegicus calcium/calmodulin-dependent protein kinase II delta subunit mRNA, partial cds.; High mobility group 1; thyroid hormone receptor alpha; Rattus norvegicus cytochrome P450 4F5 (CYP4F5) mRNA, complete cds; Insulin-like growth factor 2 receptor; Rat glucagon receptor mRNA, complete cds; Arrestin, beta 1; protease (prosome, macropain) 26S subunit, ATPase 1; R. rattus mRNA for NPY-1 receptor.; kinase domain is 450.1295; Rattus rattus mRNA for PCTAIRE3, complete cds.; R. rattus RL/IF-1 mRNA.; Arrestin, beta 2; vascular endothelial growth factor; Ras-related small GTP binding protein 3A; Adenylyl cyclase 6; LIM motif-containing protein kinase 2; This sequence comes from FIG. 1b; A2 adenosine receptor [rats, striatum, mRNA, 2141 nt].; Adrenergic receptor kinase, beta 2 (G-protein-linked receptor kinase); Arrestin, beta 1; endothelial differentiation, sphingolipid G-protein-coupled receptor, 5; immediate early gene transcription factor NGFI-B; potassium inwardly-rectifying channel, subfamily J, member 12; Rattus norvegicus calcium/calmodulin-dependent protein kinase 11 delta subunit mRNA, partial cds.; interleukin 18; Max interacting protein 1; prostaglandin F2 receptor negative regulator; BRbeta B-regulatory subunit of protein phosphatase 2A; Protein tyrosine phosphatase, non-receptor type substrate 1 (SHP substrate 1); Rattus norvegicus insulin-regulated membrane aminopeptidase IRAP mRNA, complete cds; Ceruloplasmin (ferroxidase); cyclin-dependent kinase 5; adrenergic receptor kinase, beta 1; MAD (mothers against decapentaplegic, Drosophila) homolog 1; CamK I; calcium/calmodulin-dependent protein kinase type I +CaM-like protein kinase; Calcium channel alpha 1A; phosphofructokinase, muscle; p32-subunit of replication protein A; Rattus norvegicus mRNA for Janus protein tyrosine kinase 1, JAK1.; Rat insulin-like growth factor binding protein (rIGFBP-6) mRNA, complete cds.; Discoidin domain receptor (neurotrophic tyrosine kinase, receptor, type 4 (cell adhesion kinase)); Insulin-like growth factor 1 receptor; Tumor protein p53 (Li-Fraumeni syndrome); phospholipase A2, group VI; solute carrier family 2 (facilitated glucose transporter), member 5; Inhibitor of DNA binding 2, dominant negative helix-loop-helix protein; Rat mRNA for proteasome activator rPA28 subunit alpha, complete cds.; Protein tyrosine phosphatase, receptor type, A; aminopeptidase B; Rat mRNA for cyclin D1, complete cds.; syntaxin Sa; Natriuretic peptide receptor A/Guanylate cyclase A; TR4 orphan receptor; galanin receptor 2; casein kinase II, alpha 1 polypeptide; carcinoembryonic antigen-related cell adhesion molecule; protein tyrosine phosphatase, receptor type, R; Neurofibromatosis type 1; Rat glutathione S-transferase mRNA, complete cds; calcium channel, voltage-dependent, L type, alpha 1 D subunit; Acetylcholine receptor alpha 3 (neuronal nicotine); mitogen activated protein kinase 3; mismatch repair protein; tissue inhibitor of metalloproteinase 2; Solute carrier family 4, member 2, anion exchange protein 2; Rat mRNA for multicatalytic proteionase (MCP) subunit L ingensin, Atp-dependent proteinase, proteasome, macropain).; Janus kinase 2 (a protein tyrosine kinase); kinase domain is 450..1295; Rattus rattus mRNA for PCTAIRE3, complete cds.; This sequence comes from FIG. 1; Na+/Cl(−)-dependent neurotransmitter transporter [rats, brain, mRNA, 3762 nt].; Set beta isoform; leukemogenesis protein; This sequence comes from FIG. 1 IIB; set=Set beta isoform {alternatively spliced} [rats, neonatal kidney, mRNA, 2026 nt].; synapsin II; Calmodulin III; subunit 8; R. rattus mRNA for glutathione transferase subunit 8.; Rattus norvegicus neuron-specific enolase (NSE) mRNA, complete cds; syntaxin 3; Tyrosine 3-monooxygenase/tryptophan 5monooxygenase activation protein, eta polypeptide; Carnitine palmitoyltransferase 1 alpha, liver isoform; Superoxide dismutase 1, soluble; phospholipase C, beta 3; Angiotensin I-converting enzyme (Dipeptidyl carboxypeptidase 1); c-fos protein (AA 1-380); Rat c-fos mRNA.; transmembrane receptor Unc5H2; GTPase Rab14; ATP-binding cassette, sub-family C (CFTR/MRP), member 1 (multiple drug resistance-associated protein); Inhibitor of DNA binding 1, helix-loop-helix protein (splice variation); and dipeptidylpeptidase 6. The NM protein is expressed in the patient and neuronal cell death in the patient is thereby stimulated.

An eighth embodiment of the invention is a method of screening for neuronal cell death in a patient. An NM protein is detected in a body fluid collected from the patient. The NM protein is selected from the group consisting of Ceruloplasmin (ferroxidase); Adenylyl cyclase 6; Insulin-like growth factor 1 receptor; vascular endothelial growth factor; Rat mRNA for sucrase isomaltase (EC 3.2.1.10).; Serotonin (5-hydroxytryptamine (5HT)) receptor, type 1B; Fos like antigen 2; phospholipase C-III; Rat phospholipase C-III mRNA, complete cds.; interleukin 18; calcium channel, voltage-dependent, alpha2/delta subunit 1; Vesicle-associated membrane protein (synaptobrevin 2); putative; Rattus norvegicus G protein-coupled receptor (GPR19) gene, partial cds.; This sequence comes from FIG. 1b; A2 adenosine receptor [rats, striatum, mRNA, 2141 nt].; Max interacting protein 1; ATPase isoform 2, Na+K+ transporting, beta polypeptide 2; Secretory granule neuroendocrine, protein 1 (7B2 protein); Pim-1 oncogene; adenylate kinase 3; alpha-methylacyl-CoA racemase; Inhibitor of DNA binding 2, dominant negative helix-loop-helix protein; Rattus norvegicus mRNA for 20-alpha-hydroxysteroid dehydrogenase (20-alpha-HSD), complete cds; telomerase protein component 1; pyruvate dehydrogenase kinase, isoenzyme 1; Solute carrier family 4, member 2, anion exchange protein 2; phospholipase A2, group IIC; syntaxin 3; Rattus norvegicus mRNA for interleukin-4 receptor (soluble form), complete cds; Protein tyrosine phosphatase, non-receptor type substrate 1 (SHP substrate 1); B-cell translocation gene 2, anti-proliferative; Acyl-Coa dehydrogenase, Very long chain; Clusterin; syntaxin 4; Natriuretic peptide receptor A/Guanylate cyclase A; megakaryocyte-associated tyrosine kinase; presenilin-2; phospholipase A2, group VI; pancreatic lipase-related protein 2; phospholipase C, beta 3; Phospholipase C, gamma 1; Ephrin B1; Retinoblastoma-related gene; protein kinase C epsilon subspecies; Rat protein kinase C epsilon subspecies.; Spinocerebellar ataxia type 1; phospholipase A2, group V; Angiotensin I-converting enzyme (Dipeptidyl carboxypeptidase 1); Steroid sulfatase; protein kinase C zeta subspecies; Rat protein kinase C zeta subspecies.; Calcium channel alpha 1A; carcinoembryonic antigen-related cell adhesion molecule; amphiphysin; Rat glutathione S-transferase mRNA, complete cds; Cathepsin L; Acyl Coenzyme A dehydrogenase, long chain; ATP-binding cassette, sub-family B (MDR/TAP), member 1 (P-glycoprotein/multidrug resistance 1); c-fos protein (AA 1-380); Rat c-fos mRNA.; glutamate receptor, ionotropic, AMPA2 (alpha 2); syntaxin 6; dipeptidylpeptidase 6; G protein-coupled receptor kinase 2, groucho gene related (Drosophila); Max; protein kinase C alpha (AA 1-672); Rat mRNA for protein kinase C alpha.; fatty acid amide hydrolase; Carnitine palmitoyltransferase 1 alpha, liver isoform; calcium channel, voltage-dependent, L type, alpha 1D subunit; BRbeta B-regulatory subunit of protein phosphatase 2A; Secretogranin II; transmembrane receptor Unc5H2; potassium inwardly-rectifying channel, subfamily J, member 12; Acetylcholine receptor beta; B-cell translocation gene 1, anti-proliferative; Lectin, galactose binding, soluble 9 (Galectin-9); Insulin receptor; synaptotagmin 5; Rattus norvegicus calcium/calmodulin-dependent protein kinase II delta subunit mRNA, partial cds.; High mobility group 1; thyroid hormone receptor alpha; Rattus norvegicus cytochrome P450 4F5 (CYP4F5) mRNA, complete cds; Insulin-like growth factor 2 receptor; Rat glucagon receptor mRNA, complete cds; Arrestin, beta 1; protease (prosome, macropain) 26S subunit, ATPase 1; R. rattus mRNA for NPY-1 receptor.; kinase domain is 450..1295; Rattus rattus mRNA for PCTAIRE3, complete cds.; R. rattus RL/IF-1 mRNA.; Arrestin, beta 2; vascular endothelial growth factor; Ras-related small GTP binding protein 3A; Adenylyl cyclase 6; LIM motif-containing protein kinase 2; This sequence comes from FIG. 1b; A2 adenosine receptor [rats, striatum, mRNA, 2141 nt].; Adrenergic receptor kinase, beta 2 (G-protein-linked receptor kinase); Arrestin, beta 1; endothelial differentiation, sphingolipid G-protein-coupled receptor, 5; immediate early gene transcription factor NGFI-B; potassium inwardly-rectifying channel, subfamily J, member 12; Rattus norvegicus calcium/calmodulin-dependent protein kinase II delta subunit mRNA, partial cds.; interleukin 18; Max interacting protein 1; prostaglandin F2 receptor negative regulator; BRbeta B-regulatory subunit of protein phosphatase 2A; Protein tyrosine phosphatase, non-receptor type substrate 1 (SHP substrate 1); Rattus norvegicus insulin-regulated membrane aminopeptidase IRAP mRNA, complete cds; Ceruloplasmin (ferroxidase); cyclin-dependent kinase 5; adrenergic receptor kinase, beta 1; MAD (mothers against decapentaplegic, Drosophila) homolog 1; CamK I; calcium/calmodulin-dependent protein kinase type I +CaM-like protein kinase; Calcium channel alpha 1A; phosphofructokinase, muscle; p32-subunit of replication protein A; Rattus norvegicus mRNA for Janus protein tyrosine kinase 1, JAK1.; Rat insulin-like growth factor binding protein (rIGFBP-6) mRNA, complete cds.; Discoidin domain receptor (neurotrophic tyrosine kinase, receptor, type 4 (cell adhesion kinase)); Insulin-like growth factor 1 receptor; Tumor protein p53 (Li-Fraumeni syndrome); phospholipase A2, group VI; solute carrier family 2 (facilitated glucose transporter), member 5; Inhibitor of DNA binding 2, dominant negative helix-loop-helix protein; Rat mRNA for proteasome activator rPA28 subunit alpha, complete cds.; Protein tyrosine phosphatase, receptor type, A; aminopeptidase B; Rat mRNA for cyclin D1, complete cds.; syntaxin Sa; Natriuretic peptide receptor A/Guanylate cyclase A; TR4 orphan receptor; galanin receptor 2; casein kinase II, alpha 1 polypeptide; carcinoembryonic antigen-related cell adhesion molecule; protein tyrosine phosphatase, receptor type, R; Neurofibromatosis type 1; Rat glutathione S-transferase mRNA, complete cds; calcium channel, voltage-dependent, L type, alpha 1D subunit; Acetylcholine receptor alpha 3 (neuronal nicotine); mitogen activated protein kinase 3; mismatch repair protein; tissue inhibitor of metalloproteinase 2; Solute carrier family 4, member 2, anion exchange protein 2; Rat mRNA for multicatalytic proteionase (MCP) subunit L ingensin, Atp-dependent proteinase, proteasome, macropain).; Janus kinase 2 (a protein tyrosine kinase); kinase domain is 450..1295; Rattus rattus mRNA for PCTAIRE3, complete cds.; This sequence comes from FIG. 1; Na+/Cl(−)-dependent neurotransmitter transporter [rats, brain, mRNA, 3762 nt].; Set beta isoform; leukemogenesis protein; This sequence comes from FIG. 1 IIB; set=Set beta isoform {alternatively spliced} [rats, neonatal kidney, mRNA, 2026 nt].; synapsin II; Calmodulin III; subunit 8; R. rattus mRNA for glutathione transferase subunit 8.; Rattus norvegicus neuron-specific enolase (NSE) mRNA, complete cds; syntaxin 3; Tyrosine 3-monooxygenase/tryptophan 5monooxygenase activation protein, eta polypeptide; Carnitine palmitoyltransferase 1 alpha, liver isoform; Superoxide dismutase 1, soluble; phospholipase C, beta 3; Angiotensin I-converting enzyme (Dipeptidyl carboxypeptidase 1); c-fos protein (AA 1-380); Rat c-fos mRNA.; transmembrane receptor Unc5H2; GTPase Rab14; ATP-binding cassette, sub-family C (CFTR/MRP), member 1 (multiple drug resistance-associated protein); Inhibitor of DNA binding 1, helix-loop-helix protein (splice variation); and dipeptidylpeptidase 6. Detection of the NM protein indicates neuronal cell death in the patient.

A ninth embodiment of the invention is a method of screening for neuronal cell death in a patient. A nucleic acid encoding an NM protein selected from the group consisting of: Ceruloplasmin (ferroxidase); Adenylyl cyclase 6; Insulin-like growth factor 1 receptor; vascular endothelial growth factor; Rat mRNA for sucrase isomaltase (EC 3.2.1.10).; Serotonin (5-hydroxytryptamine (5HT)) receptor, type 1B; Fos like antigen 2; phospholipase C-III; Rat phospholipase C-III mRNA, complete cds.; interleukin 18; calcium channel, voltage-dependent, alpha2/delta subunit 1; Vesicle-associated membrane protein (synaptobrevin 2); putative; Rattus norvegicus G protein-coupled receptor (GPR19) gene, partial cds.; This sequence comes from FIG. 1b; A2 adenosine receptor [rats, striatum, mRNA, 2141 nt].; Max interacting protein 1; ATPase isoform 2, Na+K+ transporting, beta polypeptide 2; Secretory granule neuroendocrine, protein 1 (7B2 protein); Pim-1 oncogene; adenylate kinase 3; alpha-methylacyl-CoA racemase; Inhibitor of DNA binding 2, dominant negative helix-loop-helix protein; Rattus norvegicus mRNA for 20-alpha-hydroxysteroid dehydrogenase (20-alpha-HSD), complete cds; telomerase protein component 1; pyruvate dehydrogenase kinase, isoenzyme 1; Solute carrier family 4, member 2, anion exchange protein 2; phospholipase A2, group IIC; syntaxin 3; Rattus norvegicus mRNA for interleukin-4 receptor (soluble form), complete cds; Protein tyrosine phosphatase, non-receptor type substrate 1 (SHP substrate 1); B-cell translocation gene 2, anti-proliferative; Acyl-Coa dehydrogenase, Very long chain; Clusterin; syntaxin 4; Natriuretic peptide receptor A/Guanylate cyclase A; megakaryocyte-associated tyrosine kinase; presenilin-2; phospholipase A2, group VI; pancreatic lipase-related protein 2; phospholipase C, beta 3; Phospholipase C, gamma 1; Ephrin B1; Retinoblastoma-related gene; protein kinase C epsilon subspecies; Rat protein kinase C epsilon subspecies.; Spinocerebellar ataxia type 1; phospholipase A2, group V; Angiotensin I-converting enzyme (Dipeptidyl carboxypeptidase 1); Steroid sulfatase; protein kinase C zeta subspecies; Rat protein kinase C zeta subspecies.; Calcium channel alpha 1A; carcinoembryonic antigen-related cell adhesion molecule; amphiphysin; Rat glutathione S-transferase mRNA, complete cds; Cathepsin L; Acyl Coenzyme A dehydrogenase, long chain; ATP-binding cassette, sub-family B (MDR/TAP), member 1 (P-glycoprotein/multidrug resistance 1); c-fos protein (AA 1-380); Rat c-fos mRNA.; glutamate receptor, ionotropic, AMPA2 (alpha 2); syntaxin 6; dipeptidylpepfidase 6; G protein-coupled receptor kinase 2, groucho gene related (Drosophila); Max; protein kinase C alpha (AA 1-672); Rat mRNA for protein kinase C alpha.; fatty acid amide hydrolase; Carnitine palmitoyltransferase 1 alpha, liver isoform; calcium channel, voltage-dependent, L type, alpha 1D subunit; BRbeta B-regulatory subunit of protein phosphatase 2A; Secretogranin II; transmembrane receptor Unc5H2; potassium inwardly-rectifying channel, subfamily J, member 12; Acetylcholine receptor beta; B-cell translocation gene 1, anti-proliferative; Lectin, galactose binding, soluble 9 (Galectin-9); Insulin receptor; synaptotagmin 5; Rattus norvegicus calcium/calmodulin-dependent protein kinase II delta subunit mRNA, partial cds.; High mobility group 1; thyroid hormone receptor alpha; Rattus norvegicus cytochrome P450 4F5 (CYP4F5) mRNA, complete cds; Insulin-like growth factor 2 receptor; Rat glucagon receptor mRNA, complete cds; Arrestin, beta 1; protease (prosome, macropain) 26S subunit, ATPase 1; R. rattus mRNA for NPY-1 receptor.; kinase domain is 450..1295; Rattus rattus mRNA for PCTAIRE3, complete cds.; R. rattus RL/IF-1 mRNA.; Arrestin, beta 2; vascular endothelial growth factor; Ras-related small GTP binding protein 3A; Adenylyl cyclase 6; LIM motif-containing protein kinase 2; This sequence comes from FIG. 1b; A2 adenosine receptor [rats, striatum, mRNA, 2141 nt].; Adrenergic receptor kinase, beta 2 (G-protein-linked receptor kinase); Arrestin, beta 1; endothelial differentiation, sphingolipid G-protein-coupled receptor, 5; immediate early gene transcription factor NGFI-B; potassium inwardly-rectifying channel, subfamily J, member 12; Rattus norvegicus calcium/calmodulin-dependent protein kinase II delta subunit mRNA, partial cds.; interleukin 18; Max interacting protein 1; prostaglandin F2 receptor negative regulator; BRbeta B-regulatory subunit of protein phosphatase 2A; Protein tyrosine phosphatase, non-receptor type substrate 1 (SHP substrate 1); Rattus norvegicus insulin-regulated membrane aminopeptidase IRAP mRNA, complete cds; Ceruloplasmin (ferroxidase); cyclin-dependent kinase 5; adrenergic receptor kinase, beta 1; MAD (mothers against decapentaplegic, Drosophila) homolog 1; CamK I; calcium/calmodulin-dependent protein kinase type I +CaM-like protein kinase; Calcium channel alpha 1A; phosphofructokinase, muscle; p32-subunit of replication protein A; Rattus norvegicus mRNA for Janus protein tyrosine kinase 1, JAK1.; Rat insulin-like growth factor binding protein (rIGFBP-6) mRNA, complete cds.; Discoidin domain receptor (neurotrophic tyrosine kinase, receptor, type 4 (cell adhesion kinase)); Insulin-like growth factor 1 receptor; Tumor protein p53 (Li-Fraumeni syndrome); phospholipase A2, group VI; solute carrier family 2 (facilitated glucose transporter), member 5; Inhibitor of DNA binding 2, dominant negative helix-loop-helix protein; Rat mRNA for proteasome activator rPA28 subunit alpha, complete cds.; Protein tyrosine phosphatase, receptor type, A; aminopeptidase B; Rat mRNA for cyclin D1, complete cds.; syntaxin 5a; Natriuretic peptide receptor A/Guanylate cyclase A; TR4 orphan receptor; galanin receptor 2; casein kinase II, alpha I polypeptide; carcinoembryonic antigen-related cell adhesion molecule; protein tyrosine phosphatase, receptor type, R; Neurofibromatosis type 1; Rat glutathione S-transferase mRNA, complete cds; calcium channel, voltage-dependent, L type, alpha 1D subunit; Acetylcholine receptor alpha 3 (neuronal nicotine); mitogen activated protein kinase 3; mismatch repair protein; tissue inhibitor of metalloproteinase 2; Solute carrier family 4, member 2, anion exchange protein 2; Rat mRNA for multicatalytic proteionase (MCP) subunit L ingensin, Atp-dependent proteinase, proteasome, macropain).; Janus kinase 2 (a protein tyrosine kinase); kinase domain is 450..1295; Rattus rattus mRNA for PCTAIRE3, complete cds.; This sequence comes from FIG. 1; Na+/Cl(−)-dependent neurotransmitter transporter [rats, brain, mRNA, 3762 nt].; Set beta isoform; leukemogenesis protein; This sequence comes from FIG. 1 IIB; set=Set beta isoform {alternatively spliced} [rats, neonatal kidney, mRNA, 2026 nt].; synapsin II; Calmodulin III; subunit 8; R. rattus mRNA for glutathione transferase subunit 8.; Rattus norvegicus neuron-specific enolase (NSE) mRNA, complete cds; syntaxin 3; Tyrosine 3-monooxygenase/tryptophan 5-monooxygenase activation protein, eta polypeptide; Carnitine palmitoyltransferase 1 alpha, liver isoform; Superoxide dismutase 1, soluble; phospholipase C, beta 3; Angiotensin I-converting enzyme (Dipeptidyl carboxypeptidase 1); c-fos protein (AA 1-380); Rat c-fos mRNA.; transmembrane receptor Unc5H2; GTPase Rab14; ATP-binding cassette, sub-family C (CFTR/MRP), member 1 (multiple drug resistance-associated protein); Inhibitor of DNA binding 1, helix-loop-helix protein (splice variation); and dipeptidylpeptidase 6 is detected in a body fluid of the patient. Detection of the NM protein indicates neuronal cell death in the patient.

A tenth embodiment of the invention is a method to identify candidate drugs for treating neuronal cell death. Cells which express one or more NM genes are contacted with a test compound. The NM genes are selected from the group consisting of Ceruloplasmin (ferroxidase); Adenylyl cyclase 6; Insulin-like growth factor 1 receptor; vascular endothelial growth factor; Rat mRNA for sucrase isomaltase (EC 3.2.1.10).; Serotonin (5-hydroxytryptamine (5HT)) receptor, type 1B; Fos like antigen 2; phospholipase C-III; Rat phospholipase C-III mRNA, complete cds.; interleukin 18; calcium channel, voltage-dependent, alpha2/delta subunit 1; Vesicle-associated membrane protein (synaptobrevin 2); putative; Rattus norvegicus G protein-coupled receptor (GPR19) gene, partial cds.; This sequence comes from FIG. 1b; A2 adenosine receptor [rats, striatum, mRNA, 2141 nt].; Max interacting protein 1; ATPase isoform 2, Na+K+ transporting, beta polypeptide 2; Secretory granule neuroendocrine, protein 1 (7B2 protein); Pim-1 oncogene; adenylate kinase 3; alpha-methylacyl-CoA racemase; Inhibitor of DNA binding 2, dominant negative helix-loop-helix protein; Rattus norvegicus mRNA for 20-alpha-hydroxysteroid dehydrogenase (20-alpha-HSD), complete cds; telomerase protein component 1; pyruvate dehydrogenase kinase, isoenzyme 1; Solute carrier family 4, member 2, anion exchange protein 2; phospholipase A2, group IIC; syntaxin 3; Rattus norvegicus mRNA for interleukin-4 receptor (soluble form), complete cds; Protein tyrosine phosphatase, non-receptor type substrate 1 (SHP substrate 1); B-cell translocation gene 2, anti-proliferative; Acyl-Coa dehydrogenase, Very long chain; Clusterin; syntaxin 4; Natriuretic peptide receptor A/Guanylate cyclase A; megakaryocyte-associated tyrosine kinase; presenilin-2; phospholipase A2, group VI; pancreatic lipase-related protein 2; phospholipase C, beta 3; Phospholipase C, gamma 1; Ephrin B1; Retinoblastoma-related gene; protein kinase C epsilon subspecies; Rat protein kinase C epsilon subspecies.; Spinocerebellar ataxia type 1; phospholipase A2, group V; Angiotensin I-converting enzyme (Dipeptidyl carboxypeptidase 1); Steroid sulfatase; protein kinase C zeta subspecies; Rat protein kinase C zeta subspecies.; Calcium channel alpha 1A; carcinoembryonic antigen-related cell adhesion molecule; amphiphysin; Rat glutathione S-transferase mRNA, complete cds; Cathepsin L; Acyl Coenzyme A dehydrogenase, long chain; ATP-binding cassette, sub-family B (MDR/TAP), member 1 (P-glycoprotein/multidrug resistance 1); c-fos protein (AA 1-380); Rat c-fos mRNA.; glutamate receptor, ionotropic, AMPA2 (alpha 2); syntaxin 6; dipeptidylpeptidase 6; G protein-coupled receptor kinase 2, groucho gene related (Drosophila); Max; protein kinase C alpha (AA 1-672); Rat mRNA for protein kinase C alpha.; fatty acid amide hydrolase; Carnitine palmitoyltransferase 1 alpha, liver isoform; calcium channel, voltage-dependent, L type, alpha 1D subunit; BRbeta B-regulatory subunit of protein phosphatase 2A; Secretogranin II; transmembrane receptor Unc5H2; potassium inwardly-rectifying channel, subfamily J, member 12; Acetylcholine receptor beta; B-cell translocation gene 1, anti-proliferative; Lectin, galactose binding, soluble 9 (Galectin-9); Insulin receptor; synaptotagmin 5; Rattus norvegicus calcium/calmodulin-dependent protein kinase II delta subunit mRNA, partial cds.; High mobility group 1; thyroid hormone receptor alpha; Rattus norvegicus cytochrome P450 4F5 (CYP4F5) mRNA, complete cds; Insulin-like growth factor 2 receptor; Rat glucagon receptor mRNA, complete cds; Arrestin, beta 1; protease (prosome, macropain) 26S subunit, ATPase 1; R. rattus mRNA for NPY-1 receptor.; kinase domain is 450..1295; Rattus rattus mRNA for PCTAIRE3, complete cds.; R. rattus RL/IF-1 mRNA.; Arrestin, beta 2; vascular endothelial growth factor; Ras-related small GTP binding protein 3A; Adenylyl cyclase 6; LIM motif-containing protein kinase 2; This sequence comes from FIG. 1b; A2 adenosine receptor [rats, striatum, mRNA, 2141 nt].; Adrenergic receptor kinase, beta 2 (G-protein-linked receptor kinase); Arrestin, beta 1; endothelial differentiation, sphingolipid G-protein-coupled receptor, 5; immediate early gene transcription factor NGFI-B; potassium inwardly-rectifying channel, subfamily J, member 12; Rattus norvegicus calcium/calmodulin-dependent protein kinase II delta subunit mRNA, partial cds.; interleukin 18; Max interacting protein 1; prostaglandin F2 receptor negative regulator; BRbeta B-regulatory subunit of protein phosphatase 2A; Protein tyrosine phosphatase, non-receptor type substrate 1 (SHP substrate 1); Rattus norvegicus insulin-regulated membrane aminopeptidase IRAP mRNA, complete cds; Ceruloplasmin (ferroxidase); cyclin-dependent kinase 5; adrenergic receptor kinase, beta 1; MAD (mothers against decapentaplegic, Drosophila) homolog 1; CamK I; calcium/calmodulin-dependent protein kinase type I +CaM-like protein kinase; Calcium channel alpha 1A; phosphofructokinase, muscle; p32-subunit of replication protein A; Rattus norvegicus mRNA for Janus protein tyrosine kinase 1, JAK1.; Rat insulin-like growth factor binding protein (rIGFBP-6) mRNA, complete cds.; Discoidin domain receptor (neurotrophic tyrosine kinase, receptor, type 4 (cell adhesion kinase)); Insulin-like growth factor 1 receptor; Tumor protein p53 (Li-Fraumeni syndrome); phospholipase A2, group VI; solute carrier family 2 (facilitated glucose transporter), member 5; Inhibitor of DNA binding 2, dominant negative helix-loop-helix protein; Rat mRNA for proteasome activator rPA28 subunit alpha, complete cds.; Protein tyrosine phosphatase, receptor type, A; aminopeptidase B; Rat mRNA for cyclin D1, complete cds.; syntaxin 5a; Natriuretic peptide receptor A/Guanylate cyclase A; TR4 orphan receptor; galanin receptor 2; casein kinase II, alpha 1 polypeptide; carcinoembryonic antigen-related cell adhesion molecule; protein tyrosine phosphatase, receptor type, R; Neurofibromatosis type 1; Rat glutathione S-transferase mRNA, complete cds; calcium channel, voltage-dependent, L type, alpha 1D subunit; Acetylcholine receptor alpha 3 (neuronal nicotine); mitogen activated protein kinase 3; mismatch repair protein; tissue inhibitor of metalloproteinase 2; Solute carrier family 4, member 2, anion exchange protein 2; Rat mRNA for multicatalytic proteionase (MCP) subunit L ingensin, Atp-dependent proteinase, proteasome, macropain).; Janus kinase 2 (a protein tyrosine kinase); kinase domain is 450..1295; Rattus rattus mRNA for PCTAIRE3, complete cds.; This sequence comes from FIG. 1; Na+/Cl(−)-dependent neurotransmitter transporter [rats, brain, mRNA, 3762 nt].; Set beta isoform; leukemogenesis protein; This sequence comes from FIG. 1 IIB; set=Set beta isoform {alternatively spliced} [rats, neonatal kidney, mRNA, 2026 nt].; synapsin II; Calmodulin III; subunit 8; R. rattus mRNA for glutathione transferase subunit 8.; Rattus norvegicus neuron-specific enolase (NSE) mRNA, complete cds; syntaxin 3; Tyrosine 3-monooxygenase/tryptophan 5monooxygenase activation protein, eta polypeptide; Carnitine palmitoyltransferase 1 alpha, liver isoform; Superoxide dismutase 1, soluble; phospholipase C, beta 3; Angiotensin I-converting enzyme (Dipeptidyl carboxypeptidase 1); c-fos protein (AA 1-380); Rat c-fos mRNA.; transmembrane receptor Unc5H2; GTPase Rab14; ATP-binding cassette, sub-family C (CFTR/MRP), member 1 (multiple drug resistance-associated protein); Inhibitor of DNA binding 1, helix-loop-helix protein (splice variation); and dipeptidylpeptidase 6. Expression of said one or more NM genes is detected by hybridization of mRNA of said cells to a nucleic acid probe which is complementary to said mRNA. A test compound is identified as a candidate drug for treating neuronal cell death if it decreases expression of said one or more NM genes.

An eleventh embodiment of the invention is a method to identify candidate drugs for treating neuronal cell death. Cells which express one or more NM proteins are contacted with a test compound. The NM proteins are selected from the group consisting of: Ceruloplasmin (ferroxidase); Adenylyl cyclase 6; Insulin-like growth factor 1 receptor; vascular endothelial growth factor; Rat mRNA for sucrase isomaltase (EC 3.2.1.10).; Serotonin (5-hydroxytryptamine (5HT)) receptor, type 1B; Fos like antigen 2; phospholipase C-III; Rat phospholipase C-III mRNA, complete cds.; interleukin 18; calcium channel, voltage-dependent, alpha2/delta subunit 1; Vesicle-associated membrane protein (synaptobrevin 2); putative; Rattus norvegicus G protein-coupled receptor (GPR19) gene, partial cds.; This sequence comes from FIG. 1b; A2 adenosine receptor [rats, striatum, mRNA, 2141 nt].; Max interacting protein 1; ATPase isoform 2, Na+K+ transporting, beta polypeptide 2; Secretory granule neuroendocrine, protein 1 (7B2 protein); Pim-1 oncogene; adenylate kinase 3; alpha-methylacyl-CoA racemase; Inhibitor of DNA binding 2, dominant negative helix-loop-helix protein; Rattus norvegicus mRNA for 20-alpha-hydroxysteroid dehydrogenase (20-alpha-HSD), complete cds; telomerase protein component 1; pyruvate dehydrogenase kinase, isoenzyme 1; Solute carrier family 4, member 2, anion exchange protein 2; phospholipase A2, group IIC; syntaxin 3; Rattus norvegicus mRNA for interleukin-4 receptor (soluble form), complete cds; Protein tyrosine phosphatase, non-receptor type substrate 1 (SHP substrate 1); B-cell translocation gene 2, anti-proliferative; Acyl-Coa dehydrogenase, Very long chain; Clusterin; syntaxin 4; Natriuretic peptide receptor A/Guanylate cyclase A; megakaryocyte-associated tyrosine kinase; presenilin-2; phospholipase A2, group VI; pancreatic lipase-related protein 2; phospholipase C, beta 3; Phospholipase C, gamma 1; Ephrin B1; Retinoblastoma-related gene; protein kinase C epsilon subspecies; Rat protein kinase C epsilon subspecies.; Spinocerebellar ataxia type 1; phospholipase A2, group V; Angiotensin I-converting enzyme (Dipeptidyl carboxypeptidase 1); Steroid sulfatase; protein kinase C zeta subspecies; Rat protein kinase C zeta subspecies.; Calcium channel alpha 1A; carcinoembryonic antigen-related cell adhesion molecule; amphiphysin; Rat glutathione S-transferase mRNA, complete cds; Cathepsin L; Acyl Coenzyme A dehydrogenase, long chain; ATP-binding cassette, sub-family B (MDR/TAP), member 1 (P-glycoprotein/multidrug resistance 1); c-fos protein (AA 1-380); Rat c-fos mRNA.; glutamate receptor, ionotropic, AMPA2 (alpha 2); syntaxin 6; dipeptidylpeptidase 6; G protein-coupled receptor kinase 2, groucho gene related (Drosophila); Max; protein kinase C alpha (AA 1-672); Rat mRNA for protein kinase C alpha.; fatty acid amide hydrolase; Carnitine palmitoyltransferase 1 alpha, liver isoform; calcium channel, voltage-dependent, L type, alpha 1D subunit; BRbeta B-regulatory subunit of protein phosphatase 2A; Secretogranin II; transmembrane receptor Unc5H2; potassium inwardly-rectifying channel, subfamily J, member 12; Acetylcholine receptor beta; B-cell translocation gene 1, anti-proliferative; Lectin, galactose binding, soluble 9 (Galectin-9); Insulin receptor; synaptotagmin 5; Rattus norvegicus calcium/calmodulin-dependent protein kinase II delta subunit mRNA, partial cds.; High mobility group 1; thyroid hormone receptor alpha; Rattus norvegicus cytochrome P450 4F5 (CYP4F5) mRNA, complete cds; Insulin-like growth factor 2 receptor; Rat glucagon receptor mRNA, complete cds; Arrestin, beta 1; protease (prosome, macropain) 26S subunit, ATPase 1; R. rattus mRNA for NPY-1 receptor.; kinase domain is 450..1295; Rattus rattus mRNA for PCTAIRE3, complete cds.; R. rattus RL/IF-1 mRNA.; Arrestin, beta 2; vascular endothelial growth factor; Ras-related small GTP binding protein 3A; Adenylyl cyclase 6; LIM motif-containing protein kinase 2; This sequence comes from FIG. 1b; A2 adenosine receptor [rats, striatum, mRNA, 2141 nt].; Adrenergic receptor kinase, beta 2 (G-protein-linked receptor kinase); Arrestin, beta 1; endothelial differentiation, sphingolipid G-protein-coupled receptor, 5; immediate early gene transcription factor NGFI-B; potassium inwardly-rectifying channel, subfamily J, member 12; Rattus norvegicus calcium/calmodulin-dependent protein kinase II delta subunit mRNA, partial cds.; interleukin 18; Max interacting protein 1; prostaglandin F2 receptor negative regulator; BRbeta B-regulatory subunit of protein phosphatase 2A; Protein tyrosine phosphatase, non-receptor type substrate 1 (SHP substrate 1); Rattus norvegicus insulin-regulated membrane aminopeptidase IRAP mRNA, complete cds; Ceruloplasmin (ferroxidase); cyclin-dependent kinase 5; adrenergic receptor kinase, beta 1; MAD (mothers against decapentaplegic, Drosophila) homolog 1; CamK I; calcium/calmodulin-dependent protein kinase type I +CaM-like protein kinase; Calcium channel alpha 1A; phosphofructokinase, muscle; p32-subunit of replication protein A; Rattus norvegicus mRNA for Janus protein tyrosine kinase 1, JAK1.; Rat insulin-like growth factor binding protein (rIGFBP-6) mRNA, complete cds.; Discoidin domain receptor (neurotrophic tyrosine kinase, receptor, type 4 (cell adhesion kinase)); Insulin-like growth factor 1 receptor; Tumor protein p53 (Li-Fraumeni syndrome); phospholipase A2, group VI; solute carrier family 2 (facilitated glucose transporter), member 5; Inhibitor of DNA binding 2, dominant negative helix-loop-helix protein; Rat mRNA for proteasome activator rPA28 subunit alpha, complete cds.; Protein tyrosine phosphatase, receptor type, A; aminopeptidase B; Rat mRNA for cyclin D1, complete cds.; syntaxin 5a; Natriuretic peptide receptor A/Guanylate cyclase A; TR4 orphan receptor; galanin receptor 2; casein kinase II, alpha 1 polypeptide; carcinoembryonic antigen-related cell adhesion molecule; protein tyrosine phosphatase, receptor type, R; Neurofibromatosis type 1; Rat glutathione S-transferase mRNA, complete cds; calcium channel, voltage-dependent, L type, alpha 1D subunit; Acetylcholine receptor alpha 3 (neuronal nicotine); mitogen activated protein kinase 3; mismatch repair protein; tissue inhibitor of metalloproteinase 2; Solute carrier family 4, member 2, anion exchange protein 2; Rat mRNA for multicatalytic proteionase (MCP) subunit L ingensin, Atp-dependent proteinase, proteasome, macropain).; Janus kinase 2 (a protein tyrosine kinase); kinase domain is 450..1295; Rattus rattus mRNA for PCTAIRE3, complete cds.; This sequence comes from FIG. 1; Na+/Cl(−)-dependent neurotransmitter transporter [rats, brain, mRNA, 3762 nt].; Set beta isoform; leukemogenesis protein; This sequence comes from FIG. 1 IIB; set=Set beta isoform {alternatively spliced} [rats, neonatal kidney, mRNA, 2026 nt].; synapsin II; Calmodulin III; subunit 8; R. rattus mRNA for glutathione transferase subunit 8.; Rattus norvegicus neuron-specific enolase (NSE) mRNA, complete cds; syntaxin 3; Tyrosine 3-monooxygenase/tryptophan 5-monooxygenase activation protein, eta polypeptide; Carnitine palmitoyltransferase 1 alpha, liver isoform; Superoxide dismutase 1, soluble; phospholipase C, beta 3; Angiotensin I-converting enzyme (Dipeptidyl carboxypeptidase 1); c-fos protein (AA 1-380); Rat c-fos mRNA.; transmembrane receptor Unc5H2; GTPase Rab14; ATP-binding cassette, sub-family C (CFTR/MRP), member 1 (multiple drug resistance-associated protein); Inhibitor of DNA binding 1, helix-loop-helix protein (splice variation); and dipeptidylpeptidase 6. The amount of said one or more NM proteins in said cells is determined. A test compound is identified as a candidate drug for treating tumors if it decreases the amount of one or more NM proteins in said cells.

An eleventh embodiment of the invention is a method to identify candidate drugs for treating neuronal cell death. Cells which express one or more NM proteins are contacted with a test compound. The NM proteins are selected from the group consisting of: Ceruloplasmin (ferroxidase); Adenylyl cyclase 6; Insulin-like growth factor 1 receptor; vascular endothelial growth factor; Rat mRNA for sucrase isomaltase (EC 3.2.1.10).; Serotonin (5-hydroxytryptamine (5HT)) receptor, type 1B; Fos like antigen 2; phospholipase C-III; Rat phospholipase C-III mRNA, complete cds.; interleukin 18; calcium channel, voltage-dependent, alpha2/delta subunit 1; Vesicle-associated membrane protein (synaptobrevin 2); putative; Rattus norvegicus G protein-coupled receptor (GPR19) gene, partial cds.; This sequence comes from FIG. 1b; A2 adenosine receptor [rats, striatum, mRNA, 2141 nt].; Max interacting protein 1; ATPase isoform 2, Na+K+ transporting, beta polypeptide 2; Secretory granule neuroendocrine, protein 1 (7B2 protein); Pim-1 oncogene; adenylate kinase 3; alpha-methylacyl-CoA racemase; Inhibitor of DNA binding 2, dominant negative helix-loop-helix protein; Rattus norvegicus mRNA for 20-alpha-hydroxysteroid dehydrogenase (20-alpha-HSD), complete cds; telomerase protein component 1; pyruvate dehydrogenase kinase, isoenzyme 1; Solute carrier family 4, member 2, anion exchange protein 2; phospholipase A2, group IIC; syntaxin 3; Rattus norvegicus mRNA for interleukin-4 receptor (soluble form), complete cds; Protein tyrosine phosphatase, non-receptor type substrate 1 (SHP substrate 1); B-cell translocation gene 2, anti-proliferative; Acyl-Coa dehydrogenase, Very long chain; Clusterin; syntaxin 4; Natriuretic peptide receptor A/Guanylate cyclase A; megakaryocyte-associated tyrosine kinase; presenilin-2; phospholipase A2, group VI; pancreatic lipase-related protein 2; phospholipase C, beta 3; Phospholipase C, gamma 1; Ephrin B1; Retinoblastoma-related gene; protein kinase C epsilon subspecies; Rat protein kinase C epsilon subspecies.; Spinocerebellar ataxia type 1; phospholipase A2, group V; Angiotensin I-converting enzyme (Dipeptidyl carboxypeptidase 1); Steroid sulfatase; protein kinase C zeta subspecies; Rat protein kinase C zeta subspecies.; Calcium channel alpha 1A; carcinoembryonic antigen-related cell adhesion molecule; amphiphysin; Rat glutathione S-transferase mRNA, complete eds; Cathepsin L; Acyl Coenzyme A dehydrogenase, long chain; ATP-binding cassette, sub-family B (MDR/TAP), member 1 (P-glycoprotein/multidrug resistance 1); c-fos protein (AA 1-380); Rat c-fos mRNA.; glutamate receptor, ionotropic, AMPA2 (alpha 2); syntaxin 6; dipeptidylpeptidase 6; G protein-coupled receptor kinase 2, groucho gene related (Drosophila); Max; protein kinase C alpha (AA 1-672); Rat mRNA for protein kinase C alpha.; fatty acid amide hydrolase; Carnitine palmitoyltransferase 1 alpha, liver isoform; calcium channel, voltage-dependent, L type, alpha 1D subunit; BRbeta B-regulatory subunit of protein phosphatase 2A; Secretogranin II; transmembrane receptor Unc5H2; potassium inwardly-rectifng channel, subfamily J, member 12; Acetylcholine receptor beta; B-cell translocation gene 1, anti-proliferative; Lectin, galactose binding, soluble 9 (Galectin-9); Insulin receptor; synaptotagmin 5; Rattus norvegicus calcium/calmodulin-dependent protein kinase II delta subunit mRNA, partial cds.; High mobility group 1; thyroid hormone receptor alpha; Rattus norvegicus cytochrome P450 4F5 (CYP4F5) mRNA, complete cds; Insulin-like growth factor 2 receptor; Rat glucagon receptor mRNA, complete cds; Arrestin, beta 1; protease (prosome, macropain) 26S subunit, ATPase 1; R. rattus mRNA for NPY-1 receptor.; kinase domain is 450..1295; Rattus rattus mRNA for PCTAIRE3, complete cds.; R. rattus RL/IF-1 mRNA.; Arrestin, beta 2; vascular endothelial growth factor; Ras-related small GTP binding protein 3A; Adenylyl cyclase 6; LIM motif-containing protein kinase 2; This sequence comes from FIG. 1b; A2 adenosine receptor [rats, striatum, mRNA, 2141 nt].; Adrenergic receptor kinase, beta 2 (G-protein-linked receptor kinase); Arrestin, beta 1; endothelial differentiation, sphingolipid G-protein-coupled receptor, 5; immediate early gene transcription factor NGFI-B; potassium inwardly-rectifying channel, subfamily J, member 12; Rattus norvegicus calcium/calmodulin-dependent protein kinase II delta subunit mRNA, partial cds.; interleukin 18; Max interacting protein 1; prostaglandin F2 receptor negative regulator; BRbeta B-regulatory subunit of protein phosphatase 2A; Protein tyrosine phosphatase, non-receptor type substrate 1 (SHP substrate 1); Rattus norvegicus insulin-regulated membrane aminopeptidase IRAP mRNA, complete cds; Ceruloplasmin (ferroxidase); cyclin-dependent kinase 5; adrenergic receptor kinase, beta 1; MAD (mothers against decapentaplegic, Drosophila) homolog 1; CamK I; calcium/calmodulin-dependent protein kinase type I +CaM-like protein kinase; Calcium channel alpha 1A; phosphofructokinase, muscle; p32-subunit of replication protein A; Rattus norvegicus mRNA for Janus protein tyrosine kinase 1, JAK1.; Rat insulin-like growth factor binding protein (rIGFBP-6) mRNA, complete cds.; Discoidin domain receptor (neurotrophic tyrosine kinase, receptor, type 4 (cell adhesion kinase)); Insulin-like growth factor 1 receptor; Tumor protein p53 (Li-Fraumeni syndrome); phospholipase A2, group VI; solute carrier family 2 (facilitated glucose transporter), member 5; Inhibitor of DNA binding 2, dominant negative helix-loop-helix protein; Rat mRNA for proteasome activator rPA28 subunit alpha, complete cds.; Protein tyrosine phosphatase, receptor type, A; aminopeptidase B; Rat mRNA for cyclin D1, complete cds.; syntaxin Sa; Natriuretic peptide receptor A/Guanylate cyclase A; TR4 orphan receptor; galanin receptor 2; casein kinase II, alpha 1 polypeptide; carcinoembryonic aritigen-related cell adhesion molecule; protein tyrosine phosphatase, receptor type, R; Neurofibromatosis type 1; Rat glutathione S-transferase mRNA, complete cds; calcium channel, voltage-dependent, L type, alpha 1D subunit; Acetylcholine receptor alpha 3 (neuronal nicotine); mitogen activated protein kinase 3; mismatch repair protein; tissue inhibitor of metalloproteinase 2; Solute carrier family 4, member 2, anion exchange protein 2; Rat mRNA for multicatalytic proteionase (MCP) subunit L ingensin, Atp-dependent proteinase, proteasome, macropain).; Janus kinase 2 (a protein tyrosine kinase); kinase domain is 450..1295; Rattus rattus mRNA for PCTAIRE3, complete cds.; This sequence comes from FIG. 1; Na+/Cl(−)-dependent neurotransmitter transporter [rats, brain, mRNA, 3762 nt].; Set beta isoform; leukemogenesis protein; This sequence comes from FIG. 1 IIB; set=Set beta isoform {alternatively spliced} [rats, neonatal kidney, mRNA, 2026 nt].; synapsin II; Calmodulin III; subunit 8; R. rattus mRNA for glutathione transferase subunit 8.; Rattus norvegicus neuron-specific enolase (NSE) mRNA, complete cds; syntaxin 3; Tyrosine 3-monooxygenase/tryptophan 5-monooxygenase activation protein, eta polypeptide; Carnitine palmitoyltransferase 1 alpha, liver isoform; Superoxide dismutase 1, soluble; phospholipase C, beta 3; Angiotensin I-converting enzyme (Dipeptidyl carboxypeptidase 1); c-fos protein (AA 1-380); Rat c-fos mRNA.; transmembrane receptor Unc5H2; GTPase Rab14; ATP-binding cassette, sub-family C (CFTR/MRP), member 1 (multiple drug resistance-associated protein); Inhibitor of DNA binding 1, helix-loop-helix protein (splice variation); and dipeptidylpeptidase 6. Activity of said one or more NM proteins in said cells is determined. A test compound is identified as a candidate drug for treating neuronal cell death if it decreases the activity of one more NM proteins in said cells.

A twelfth embodiment of the invention is a method to identify candidate drugs for treating neuronal cell death. Cells are contacted with a test compound. The cells express one or more NM genes selected from the group consisting of Acetylcholine receptor alpha 5; Nerve growth factor receptor, fast; Rat insulin-like growth factor binding protein (rIGFBP-6) mRNA, complete cds.; transforming growth factor, beta receptor I; taurine/beta-alanine transporter; Rat mRNA for proteasome subunit RC10-II, complete cds.; C holinergic receptor, nicotinic, alpha polypeptide 7 (neuronal nicotinic acetycholine receptor alpha 7) (bungarotoxin alpha); 6-phosphofructo-2-kinase/fructose2,6-biphosphatase 4; heterogeneous nuclear ribonucleoproteins methyltransferase-like 2 (S. cerevisiae); R. rattus mRNA for epididymal secretory glutathione peroxidase.; matrix metalloproteinase 14, membrane-inserted; cAMP response element binding protein; Solute carrier family 2 A3 (neuron glucose transporter); ATPase, Na+K+ transporting, alpha 1 polypeptide; Fyn proto-oncogene; protein kinase inhibitor, alpha; Rattus norvegicus galactosyltransferase associated kinase (GTA) mRNA, complete cds; Early growth response 1; Glutathione-S-transferase, placental enzyme pi type; neogenin; ATP synthase, H+ transporting, mitochondrial F0 complex, subunit c (subunit 9), isoform 1; 36 kDa calcium-dependent phospholipid-binding protein; This sequence comes from FIG. 1; conceptual translation differs that in published reference; calpactin 1; annexin II=36 kDa calcium-dependent phospholipid-binding protein [rats, RBL-2H3 basophilic leukemia cells, mRNA, 1362 nt].; Murine leukemia viral (v-raf-1) oncogene homolog 1 (3611-MSV); Inhibitor of DNA binding 1, helix-loop-helix protein (splice variation); alternative splicing: see also D28754; Rat mRNA for cyclin dependent kinase 2-alpha.; Tyrosine 3-monooxygenase/tryptophan 5-monooxygenase activation protein, zeta polypeptide; Solute carrier family 25, member 5 (adenine nucleotid translocator 2, fibroblast isoform (ATP-ADP carrier protein)); Dopa decarboxylase (aromatic L-amino acid decarboxylase); cadherin 22; Rat thymidine kinase mRNA, 5′ end.; Solute carrier family18 (vesicular monoamine) member 1 (chromaffin granule amine transporter); mitogen-activated protein kinase 6; R. norvegicus mRNA for Cdk-activating kinase; ADP-ribosylation factor 2; mismatch repair protein; CD24 antigen; glutamate-cysteine ligase, modifier subunit; PDZ and LIM domain 1 (elfin); casein kinase II beta subunit; Inhibitor of DNA binding 3, dominant negative helix-loop-helix protein; Rattus norvegicus Sprague-Dawley lipid-binding protein mRNA, complete cds; Rat mRNA for cyclin D1, complete cds.; Proliferating cell nuclear antigen; bone morphogenetic protein 2; VGF nerve growth factor inducible; activity regulated cytoskeletal-associated protein; Fos-like antigen 1; Cyclin G1; taurine/beta-alanine transporter; Vesicle-associated membrane protein (synaptobrevin 2); unction plakoglobin; Inhibitor of DNA binding 3, dominant negative helix-loop-helix protein; Heat shock 27 kDa protein; Solute carrier family18 (vesicular monoamine) member 1 (chromaffin granule amine transporter); mitogen-activated protein kinase 6; Interleukin 6 signal transducer; Synaptophysin; latexin; Nerve growth factor receptor, fast; 36 kDa calcium-dependent phospholipid-binding protein; This sequence comes from FIG. 1; conceptual translation differs that in published reference; calpactin 1; annexin II=36 kDa calcium-dependent phospholipid-binding protein [rats, RBL-2H3 basophilic leukemia cells, mRNA, 1362 nt].; transcription factor AP-1 (AA 1-334); Rat c-jun oncogene mRNA for transcription factor AP-1.; B-cell translocation gene 1, anti-proliferativeputative anti-proliferative factor; glycoprotein hormones, alpha subunit; Adenomatosis polyposis coli; Rattus norvegicus jun-D gene, complete cds; R. rattus mRNA for heat shock protein 70.; solute carrier family 30 (zinc transporter), member 1zinc transporter; Cathepsin L; eukaryotic initiation factor 5 (eIF-5); 3-hydroxy-3-methylglutaryl-Coenzyme A synthase 1; cysteine-rich protein 3; Solute carrier family 7 member A1 (amino acid transporter cationic 1); Cytochrom P450 Lanosterol 14 alpha-demethylase; myc box dependent interacting protein 1; plectin; ATPase, Ca++ transporting, plasma membrane 1; Rattus norvegicus Sprague-Dawley lipid-binding protein mRNA, complete cds; cyclin-dependent kinase inhibitor 1A (P21); Annexin V; bone morphogenetic protein 2; 6-phosphofructo-2kinase/fructose-2,6-biphosphatase 4; Tumor necrosis factor receptor superfamily, member 1a; ezrin; Pim-1 oncogene; Fos like antigen 2transcription factor; B-cell translocation gene 2, anti-proliferative; Rattus norvegicus RIN1 mRNA, complete cds; Rat brain glucose-transporter protein mRNA, complete cds; jun B proto-oncogene; VGF nerve growth factor inducible; Interleukin 2 receptor, beta chain; Early growth response 1; Rat mRNA for LDL-receptor; Rat mRNA for 53 kD polypeptide induced by growth factors (EGF) and oncogenes (H-ras; src; polyoma middle T); urinary plasminogen activator receptor 2urinary-type plasminogen activator receptor; Rat transformation-associated protein (34A) mRNA, complete cds; serine (or cysteine) proteinase inhibitor, clade E (nexin, plasminogen activator inhibitor type 1), member 1; Fos-like antigen 1; and activity regulated cytoskeletal-associated protein. Expression of said one or more N\M genes is detected by hybridization of mRNA of said cells to a nucleic acid probe which is complementary to said mRNA. A test compound is identified as a candidate drug for treating neuronal cell death if it increases expression of said one or more NM genes.

A thirteenth embodiment of the invention is a method for identifying candidate drugs for treating neuronal cell death. Cells which express one or more NM proteins are contacted with a test compound. The NM proteins are selected from the group consisting of: Acetylcholine receptor alpha 5; Nerve growth factor receptor, fast; Rat insulin-like growth factor binding protein (rIGFBP-6) mRNA, complete cds.; transforming growth factor, beta receptor I; taurine/beta-alanine transporter; Rat mRNA for proteasome subunit RC10-II, complete cds.; C holinergic receptor, nicotinic, alpha polypeptide 7 (neuronal nicotinic acetycholine receptor alpha 7) (bungarotoxin alpha); 6-phosphofructo-2-kinase/fructose-2,6-biphosphatase 4; heterogeneous nuclear ribonucleoproteins methyltransferase-like 2 (S. cerevisiae); R. rattus mRNA for epididymal secretory glutathione peroxidase.; matrix metalloproteinase 14, membrane-inserted; cAMP response element binding protein; Solute carrier family 2 A3 (neuron glucose transporter); ATPase, Na+K+ transporting, alpha 1 polypeptide; Fyn proto-oncogene; protein kinase inhibitor, alpha; Rattus norvegicus galactosyltransferase associated kinase (GTA) mRNA, complete cds; Early growth response 1; Glutathione-S-transferase, placental enzyme pi type; neogenin; ATP synthase, H+ transporting, mitochondrial F0 complex, subunit c (subunit 9), isoform 1; 36 kDa calcium-dependent phospholipid-binding protein; This sequence comes from FIG. 1; conceptual translation differs that in published reference; calpactin 1; annexin II=36 kDa calcium-dependent phospholipid-binding protein [rats, RBL-2H3 basophilic leukemia cells, mRNA, 1362 nt].; Murine leukemia viral (v-raf-1) oncogene homolog 1 (3611-MSV); Inhibitor of DNA binding 1, helix-loop-helix protein (splice variation); alternative splicing: see also D28754; Rat mRNA for cyclin dependent kinase 2-alpha.; Tyrosine 3-monooxygenase/tryptophan 5monooxygenase activation protein, zeta polypeptide; Solute carrier family 25, member 5 (adenine nucleotid translocator 2, fibroblast isoform (ATP-ADP carrier protein)); Dopa decarboxylase (aromatic L-amino acid decarboxylase); cadherin 22; Rat thymidine kinase mRNA, 5′ end.; Solute carrier family18 (vesicular monoamine) member 1 (chromaffin granule amine transporter); mitogen-activated protein kinase 6; R. norvegicus mRNA for Cdk-activating kinase; ADP-ribosylation factor 2; mismatch repair protein; CD24 antigen; glutamate-cysteine ligase, modifier subunit; PDZ and LIM domain 1 (elfin); casein kinase 11 beta subunit; Inhibitor of DNA binding 3, dominant negative helix-loop-helix protein; Rattus norvegicus Sprague-Dawley lipid-binding protein mRNA, complete cds; Rat mRNA for cyclin D1, complete cds.; Proliferating cell nuclear antigen; bone morphogenetic protein 2; VGF nerve growth factor inducible; activity regulated cytoskeletal-associated protein; Fos-like antigen 1; Cyclin G1; taurine/beta-alanine transporter; Vesicle-associated membrane protein (synaptobrevin 2); unction plakoglobin; Inhibitor of DNA binding 3, dominant negative helix-loop-helix protein; Heat shock 27 kDa protein; Solute carrier family18 (vesicular monoamine) member 1 (chromaffin granule amine transporter); mitogen-activated protein kinase 6; Interleukin 6 signal transducer; Synaptophysin; latexin; Nerve growth factor receptor, fast; 36 kDa calcium-dependent phospholipid-binding protein; This sequence comes from FIG. 1; conceptual translation differs that in published reference; calpactin 1; annexin II=36 kDa calcium-dependent phospholipid-binding protein [rats, RBL-2H3 basophilic leukemia cells, mRNA, 1362 nt].; transcription factor AP-1 (AA 1-334); Rat c-jun oncogene mRNA for transcription factor AP-1.; B-cell translocation gene 1, anti-proliferativeputative anti-proliferative factor; glycoprotein hormones, alpha subunit; Adenomatosis polyposis coli; Rattus norvegicus jun-D gene, complete cds; R. rattus mRNA for heat shock protein 70.; solute carrier family 30 (zinc transporter), member 1zinc transporter; Cathepsin L; eukaryotic initiation factor 5 (eIF-5); 3-hydroxy-3-methylglutaryl-Coenzyme A synthase 1; cysteine-rich protein 3; Solute carrier family 7 member A1 (amino acid transporter cationic 1); Cytochrom P450 Lanosterol 14 alpha-demethylase; myc box dependent interacting protein 1; plectin; ATPase, Ca++ transporting, plasma membrane 1; Rattus norvegicus Sprague-Dawley lipid-binding protein mRNA, complete cds; cyclin-dependent kinase inhibitor 1A (P21); Annexin V; bone morphogenetic protein 2; 6phosphofructo-2-kinase/fructose-2,6-biphosphatase 4; Tumor necrosis factor receptor superfamily, member 1a; ezrin; Pim-1 oncogene; Fos like antigen 2transcription factor; B-cell translocation gene 2, anti-proliferative; Rattus norvegicus RIN1 mRNA, complete cds; Rat brain glucose-transporter protein mRNA, complete cds; jun B proto-oncogene; VGF nerve growth factor inducible; Interleukin 2 receptor, beta chain; Early growth response 1; Rat mRNA for LDL-receptor; Rat mRNA for 53 kD polypeptide induced by growth factors (EGF) and oncogenes (H-ras; src; polyoma middle T); urinary plasminogen activator receptor 2urinary-type plasminogen activator receptor; Rat transformation-associated protein (34A) mRNA, complete cds; serine (or cysteine) proteinase inhibitor, clade E (nexin, plasminogen activator inhibitor type 1), member 1; Fos-like antigen 1; and activity regulated cytoskeletal-associated protein. The amount of said one or more NM proteins in said cells is determined. A test compound is identified as a candidate drug for treating neuronal cell death if it increases the amount of one more NM proteins in said cells.

A fourteenth embodiment of the invention is a method to identify candidate drugs for treating neuronal cell death. Cells are contacted with a test compound. The cells express one or more NM proteins selected from the group consisting of: Acetylcholine receptor alpha 5; Nerve growth factor receptor, fast; Rat insulin-like growth factor binding protein (rIGFBP-6) mRNA, complete cds.; transforming growth factor, beta receptor I; taurine/beta-alanine transporter; Rat mRNA for proteasome subunit RC10-II, complete cds.; C holinergic receptor, nicotinic, alpha polypeptide 7 (neuronal nicotinic acetycholine receptor alpha 7) (bungarotoxin alpha); 6-phosphofructo-2-kinase/fructose2,6-biphosphatase 4; heterogeneous nuclear ribonucleoproteins methyltransferase-like 2 (S. cerevisiae); R. rattus mRNA for epididymal secretory glutathione peroxidase.; matrix metalloproteinase 14, membrane-inserted; cAMP response element binding protein; Solute carrier family 2 A3 (neuron glucose transporter); ATPase, Na+K+ transporting, alpha 1 polypeptide; Fyn proto-oncogene; protein kinase inhibitor, alpha; Rattus norvegicus galactosyltransferase associated kinase (GTA) mRNA, complete cds; Early growth response 1; Glutathione-S-transferase, placental enzyme pi type; neogenin; ATP synthase, H+ transporting, mitochondrial F0 complex, subunit c (subunit 9), isoform 1; 36 kDa calcium-dependent phospholipid-binding protein; This sequence comes from FIG. 1; conceptual translation differs that in published reference; calpactin 1; annexin 1136 kDa calcium-dependent phospholipid-binding protein [rats, RBL-2H3 basophilic leukemia cells, mRNA, 1362 nt].; Murine leukemia viral (v-raf-1) oncogene homolog 1 (3611-MSV); Inhibitor of DNA binding 1, helix-loop-helix protein (splice variation); alternative splicing: see also D28754; Rat mRNA for cyclin dependent kinase 2-alpha.; Tyrosine 3-monooxygenase/tryptophan 5-monooxygenase activation protein, zeta polypeptide; Solute carrier family 25, member 5 (adenine nucleotid translocator 2, fibroblast isoform (ATP-ADP carrier protein)); Dopa decarboxylase (aromatic L-amino acid decarboxylase); cadherin 22; Rat thymidine kinase mRNA, 5′ end.; Solute carrier family18 (vesicular monoamine) member 1 (chromaffin granule amine transporter); mitogen-activated protein kinase 6; R. norvegicus mRNA for Cdk-activating kinase; ADP-ribosylation factor 2; mismatch repair protein; CD24 antigen; glutamate-cysteine ligase, modifier subunit; PDZ and LIM domain 1 (elfin); casein kinase II beta subunit; Inhibitor of DNA binding 3, dominant negative helix-loop-helix protein; Rattus norvegicus Sprague-Dawley lipid-binding protein mRNA, complete cds; Rat mRNA for cyclin D1, complete cds.; Proliferating cell nuclear antigen; bone morphogenetic protein 2; VGF nerve growth factor inducible; activity regulated cytoskeletal-associated protein; Fos-like antigen 1; Cyclin G1; taurine/beta-alanine transporter; Vesicle-associated membrane protein (synaptobrevin 2); unction plakoglobin; Inhibitor of DNA binding 3, dominant negative helix-loop-helix protein; Heat shock 27 kDa protein; Solute carrier family18 (vesicular monoamine) member 1 (chromaffin granule amine transporter); mitogen-activated protein kinase 6; Interleukin 6 signal transducer; Synaptophysin; latexin; Nerve growth factor receptor, fast; 36 kDa calcium-dependent phospholipid-binding protein; This sequence comes from FIG. 1; conceptual translation differs that in published reference; calpactin 1; annexin II=36 kDa calcium-dependent phospholipid-binding protein [rats, RBL-2H3 basophilic leukemia cells, mRNA, 1362 nt].; transcription factor AP-1 (AA 1-334); Rat c-jun oncogene mRNA for transcription factor AP-1.; B-cell translocation gene 1, anti-proliferativeputative anti-proliferative factor; glycoprotein hormones, alpha subunit; Adenomatosis polyposis coli; Rattus norvegicus jun-D gene, complete cds; R. rattus mRNA for heat shock protein 70.; solute carrier family 30 (zinc transporter), member 1zinc transporter; Cathepsin L; eukaryotic initiation factor 5 (eIF-5); 3-hydroxy-3-methylglutaryl-Coenzyme A synthase 1; cysteine-rich protein 3; Solute carrier family 7 member A1 (amino acid transporter cationic 1); Cytochrom P450 Lanosterol 14 alpha-demethylase; myc box dependent interacting protein 1; plectin; ATPase, Ca++ transporting, plasma membrane 1; Rattus norvegicus Sprague-Dawley lipid-binding protein mRNA, complete cds; cyclin-dependent kinase inhibitor 1A (P21); Annexin V; bone morphogenetic protein 2; 6-phosphofructo-2kinase/fructose-2,6-biphosphatase 4; Tumor necrosis factor receptor superfamily, member 1a; ezrin; Pim-1 oncogene; Fos like antigen 2transcription factor; B-cell translocation gene 2, anti-proliferative; Rattus norvegicus RIN1 mRNA, complete cds; Rat brain glucose-transporter protein mRNA, complete cds; jun B proto-oncogene; VGF nerve growth factor inducible; Interleukin 2 receptor, beta chain; Early growth response 1; Rat mRNA for LDL-receptor; Rat mRNA for 53 kD polypeptide induced by growth factors (EGF) and oncogenes (H-ras; src; polyoma middle T); urinary plasminogen activator receptor 2urinary-type plasminogen activator receptor; Rat transformation-associated protein (34A) mRNA, complete cds; serine (or cysteine) proteinase inhibitor, clade E (nexin, plasminogen activator inhibitor type 1), member 1; Fos-like antigen 1; and activity regulated cytoskeletal-associated protein. Activity of said one or more NM proteins in said cells is determined. A test compound is identified as a candidate drug for treating neuronal cell death if it increases the activity of one more NM proteins in said cells.

These and other embodiments which will be apparent to those of skill in the art upon reading the specification provide the art with reagents and methods for detection, diagnosis, therapy, and drug screening pertaining to neuronal cell death and pathological processes involving or requiring neuronal cell death.

### BRIEF DESCRIPTION OF THE DRAWINGS

FIG. 1 shows genes which were up regulated subsequent to serum withdrawal from PC12 cells.

FIG. 2 shows genes which were down regulated subsequent to serum withdrawal from PC12 cells.

FIG. 3 shows genes which were up regulated subsequent to NGF withdrawal from PC12 cells.

FIG. 4 shows genes which were down regulated subsequent to NGF withdrawal from PC12 cells.

### DETAILED DESCRIPTION OF THE INVENTION

We have used microarrays in analyses of gene expression of PC12 neuronal cells which were subjected to withdrawal of serum or NGF (neuronal growth factor). By comparing RNA profiles from before and after withdrawal we identified genes and molecular pathways with altered regulation that are involved in neuronal cell death.

Loss of neurons by a degenerative process is a major pathological feature of many human neurological disorders. Neuronal cell death can occur as a result of a variety of conditions including traumatic injury, ischemia, neurodegenerative diseases (e.g., Parkinson's disease, Huntington's disease, Alzheimer's disease, amyotrophic lateral sclerosis (ALS), stroke, or trauma), or as a normal part of tissue development and maintenance. Several inherited disorders produce late onset neuron loss, each of which is highly specific for particular neural cell types.

The methods of the present invention can also be applied to any of the diseases of the retina, retinal pigment epithelium (RPE), and choroid. These include, but are not limited to, ocular neovascularization, ocular inflammation and retinal degenerations. Specific examples of these disease states include diabetic retinopathy, chronic glaucoma, retinal detachment, sickle cell retinopathy, senile macular degeneration, retinal neovascularization, subretinal neovascularization; rubeosis iritis inflammatory diseases, chronic posterior and pan uveitis, neoplasms, retinoblastoma, pseudoglioma, neovascular glaucoma; neovascularization resulting following a combined vitrectomy and lensectomy, vascular diseases retinal ischemia, choroidal vascular insufficiency, choroidal thrombosis, neovascularization of the optic nerve, diabetic macular edema, cystoid macular edema, retinitis pigmentosa, retinal vein occlusion, proliferative vitreoretinopathy, angioid streak, and retinal artery occlusion, and, neovascularization due to penetration of the eye or ocular injury. Additional relevant disease include the neuropathies, such as Leber's, idiopathic, drug-induced, optic, and ischemic neropathies.

Neurodegenerative disorders more broadly can also be treated and identified using the methods of the present invention. These include disorders of the central nervous system as well as disorders of the peripheral nervous system. Neurodegenerative disorders include, but are not limited to, brain injuries, cerebrovascular diseases and their consequences, Parkinson's disease, corticobasal degeneration, motor neuron disease (including ALS), multiple sclerosis, traumatic brain injury, stroke, post-stroke, posttraumatic brain injury, and small-vessel cerebrovascular disease. Dementias, such as Alzheimer's disease, vascular dementia, dementia with Lewy bodies, frontotemporal dementia and Parkinsonism linked to chromosome 17, frontotemporal dementias (including Pick's disease), progressive nuclear palsy, corticobasal degeneration, Huntington's disease, thalamic degeneration, Creutzfeld-Jakob dementia, HIV dementia, schizophrenia with dementia, and Korsakoffs psychosis, also are neurodegenerative disorders.

Any type of neuronal cells can be used in the practice of the invention, for example, for screening for candicate drugs for treating neuronal cell death and disease resulting therefrom. Such cells include without limitation cells isolated from brain, neuroblastoma astrocytoma, glioblastoma, medulloblastoma, retinoblastoma, and retina. Such cells can be isolated as is known in the art. Cell lines of these types are available from the American Type Culture Collection, Mannassas, Va. Cells that can differentiate into neurons, such as NT2, and PC12 cells can also be used to advantage.

Isolated and purified nucleic acids, according to the present invention are those which are not linked to those genes to which they are linked in the human genome. Moreover, isolated and purified nucleic acids are not present in a mixture, such as a library, containing a multitude of distinct sequences from distinct genes. They may be, however, linked to other genes such as vector sequences or sequences of other genes to which they are not naturally adjacent. The nucleic acids may represent either the sense or the antisense strand. Nucleic acids and proteins although disclosed herein with sequence particularity may be derived from a single individual. Allelic variants which occur in the population of humans are including within the scope of such nucleic acids and proteins. Those of skill in the art are well able to identify allelic variants as being the same gene or protein.

Isolated and purified proteins are not in a cell, and are separated from the normal cellular constituents, such as nucleic acids, lipids, etc. Typically the protein is purified to such an extent that it comprises the predominant species of protein in the composition, such as greater than 50, 60 70, 80, 90, or even 95% of the proteins present.

Using the proteins according to the invention, one of ordinary skill in the art can readily generate or obtain antibodies which specifically bind to the proteins. Such antibodies can be monoclonal or polyclonal. They can be chimeric, humanized, or totally human. Any functional fragment or derivative of an antibody can be used including Fab, Fab′, Fab2, Fab′2, and single chain variable regions. So long as the fragment or derivative retains specificity of binding for the endothelial marker protein it can be used. Antibodies can be tested for specificity of binding by comparing binding to appropriate antigen to binding to irrelevant antigen or antigen mixture under a given set of conditions. If the antibody binds to the appropriate antigen at least 2, 5, 7, and preferably 10 times more than to irrelevant antigen or antigen mixture then it is considered to be specific.

Techniques for making such partially to fully human antibodies are known in the art and any such techniques can be used. According to one such technique, fully human antibody sequences are made in a transgenic mouse which has been engineered to express human heavy and light chain antibody genes. Multiple strains of such transgenic mice have been made which can produce different classes of antibodies. B cells from transgenic mice which are producing a desirable antibody can be fused to make hybridoma cell lines for continuous production of the desired antibody. See for example, Nina D. Russel, Jose R. F. Corvalan, Michael L. Gallo, C. Geoffrey Davis, Liise-Anne Pirofski. Production of Protective Human Antipneumococcal Antibodies by Transgenic Mice with Human Immunoglobulin Loci Infection and Immunity April 2000, p. 1820-1826; Michael L. Gallo, Vladimir E. Ivanov, Aya Jakobovits, and C. Geoffrey Davis. The human immunoglobulin loci introduced into mice: V (D) and J gene segment usage similar to that of adult humans European Journal of Immunology 30: 534-540, 2000; Larry L. Green. Antibody engineering via genetic engineering of the mouse: XenoMouse strains are a vehicle for the facile generation of therapeutic human monoclonal antibodies Journal of Immunological Methods 231 11-23, 1999; Yang X-D, Corvalan J R F, Wang P, Roy C M-N and Davis C G. Fully Human Anti-interleukin-8 Monoclonal Antibodies: Potential Therapeutics for the Treatment of Inflammatory Disease States. Journal of Leukocyte Biology Vol. 66, pp401-410 (1999); Yang X-D, Jia X-C, Corvalan J R F, Wang P, C G Davis and Jakobovits A. Eradication of Established Tumors by a Fully Human Monoclonal Antibody to the Epidermal Growth Factor Receptor without Concomitant Chemotherapy. Cancer Research Vol. 59, Number 6, pp1236-1243 (1999); Jakobovits A. Production and selection of antigen-specific fully human monoclonal antibodies from mice engineered with human Ig loci. Advanced Drug Delivery Reviews Vol. 31, pp: 33-42 (1998); Green L and Jakobovits A. Regulation of B cell development by variable gene complexity in mice reconstituted with human immunoglobulin yeast artificial chromosomes. J. Exp. Med. Vol. 188, Number 3, pp: 483-495 (1998); Jakobovits A. The long-awaited magic bullets: therapeutic human monoclonal antibodies from transgenic mice. Exp. Opin. Invest. Drugs Vol. 7(4), pp: 607-614 (1998); Tsuda H, Maynard-Currie K, Reid L, Yoshida T, Edamura K, Maeda N, Smithies O, Jakobovits A. Inactivation of Mouse HPRT locus by a 203-bp retrotransposon insertion and a 55-kb gene-targeted deletion: establishment of new HPRT-Deficient mouse embryonic sNM cell lines. Genomics Vol. 42, pp: 413-421 (1997); Sherman-Gold, R. Monoclonal Antibodies: The Evolution from '80s Magic Bullets To Mature, Mainstream Applications as Clinical Therapeutics. Genetic Engineering News Vol. 17, Number 14 (August 1997); Mendez M, Green L, Corvalan J, Jia X-C, Maynard-Currie C, Yang X-d, Gallo M, Louie D, Lee D, Erickson K, Luna J, Roy C, Abderrahim H, Kirschenbaum F, Noguchi M, Smith D, Fukushima A, Hales J, Finer M, Davis C, Zsebo K, Jakobovits A. Functional transplant of megabase human immunoglobulin loci recapitulates human antibody response in mice. Nature Genetics Vol. 15, pp: 146-156 (1997); Jakobovits A. Mice engineered with human immunoglobulin YACs: A new technology for production of fully human antibodies for autoimmunity therapy. Weir's Handbook of Experimental Immunology, The Integrated Immune SysNM Vol. IV, pp: 194.1-194.7 (1996); Jakobovits A. Production of fully human antibodies by transgenic mice. Current Opinion in Biotechnology Vol. 6, No. 5, pp: 561-566 (1995); Mendez M, Abderrahim H, Noguchi M, David N, Hardy M, Green L, Tsuda H, Yoast S, Maynard-Currie C, Garza D, Gemmill R, Jakobovits A, Klapholz S. Analysis of the structural integrity of YACs comprising human immunoglobulin genes in yeast and in embryonic sNM cells. Genomics Vol. 26, pp: 294-307 (1995); Jakobovits A. YAC Vectors: Humanizing the mouse genome. Current Biology Vol. 4, No. 8, pp: 761-763 (1994); Arbones M, Ord D, Ley K, Ratech H, Maynard-Curry K, Otten G, Capon D, Tedder T. Lymphocyte homing and leukocyte rolling and migration are impaired in L-selectin-deficient mice. Immunity Vol. 1, No. 4, pp: 247-260 (1994); Green L, Hardy M, Maynard-Curry K, Tsuda H, Louie D, Mendez M, Abderrahim H, Noguchi M, Smith D, Zeng Y, et. al. Antigen-specific human monoclonal antibodies from mice engineered with human Ig heavy and light chain YACs. Nature Genetics Vol. 7, No. 1, pp: 13-21 (1994); Jakobovits A, Moore A, Green L, Vergara G, Maynard-Curry K, Austin H, Klapholz S. Germ-line transmission and expression of a human-derived yeast artificial chromosome. Nature Vol. 362, No. 6417, pp: 255-258 (1993); Jakobovits A, Vergara G, Kennedy J, Hales J, McGuinness R, Casentini-Borocz D, Brenner D, Otten G. Analysis of homozygous mutant chimeric mice: deletion of the immunoglobulin heavy-chain joining region blocks B-cell development and antibody production. Proceedings of the National Academy of Sciences USA Vol. 90, No. 6, pp: 2551-2555 (1993); Kucherlapati et al., U.S. Pat. No. 6,1075,181.

Antibodies can also be made using phage display techniques. Such techniques can be used to isolate an initial antibody or to generate variants with altered specificity or avidity characteristics. Single chain Fv can also be used as is convenient. They can be made from vaccinated transgenic mice, if desired. Antibodies can be produced in cell culture, in phage, or in various animals, including but not limited to cows, rabbits, goats, mice, rats, hamsters, guinea pigs, sheep, dogs, cats, monkeys, chimpanzees, apes.

Antibodies can be labeled with a detectable moiety such as a radioactive atom, a chromophore, a fluorophore, or the like. Such labeled antibodies can be used for diagnostic techniques, either in vivo, or in an isolated test sample. Antibodies can also be conjugated, for example, to a pharmaceutical agent, such as chemotherapeutic drug or a toxin. They can be linked to a cytokine, to a ligand, to another antibody. Suitable agents for coupling to antibodies to achieve an anti-tumor effect include cytokines, such as interleukin 2 (IL-2) and Tumor Necrosis Factor (TNF); photosensitizers, for use in photodynamic therapy, including aluminum (III) phthalocyanine tetrasulfonate, hematoporphyrin, and phthalocyanine; radionuclides, such as iodine-131 (¹³¹I), yttrium-90 (⁹⁰Y), bismuth-212 (²¹²Bi), bismuth-213 (²¹³Bi), technetium-99m (^{99m}Tc), rhenium-186 (¹⁸⁶Re), and rhenium-188 (¹⁸⁸Re); antibiotics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from chinese cobra (naja naja atra), and gelonin (a plant toxin); ribosome inactivating proteins from plants, bacteria and fingi, such as restrictocin (a ribosome inactivating protein produced by Aspergillus restrictus), saporin (a ribosome inactivating protein from Saponaria officinalis), and RNase; tyrosine kinase inhibitors; ly207702 (a difluorinated purine nucleoside); liposomes containing anti-tumor agents (e.g., antisense oligonucleotides, plasmids which encode for toxins, methotrexate, etc.); and other antibodies or antibody fragments, such as F(ab).

Those of skill in the art will readily understand and be able to make such antibody derivatives, as they are well known in the art. The antibodies may be cytotoxic on their own, or they may be used to deliver cytotoxic agents to particular locations in the body. The antibodies can be administered to individuals in need thereof as a form of passive immunization.

Drugs can be screened for the ability to modulate expression of the genes, mRNA, and protein which are identified herein. Cell populations can be contacted with test substances and the expression of neuronal cell death markers determined. Test substances which decrease the expression of up-regulated neuronal cell death markers are candidates for inhibiting neuronal cell death. Conversely, test substances which increase the expression of down-regulated neuronal cell death markers can be identified as candidate drugs for causing neuronal cell death. In cases where a biological or enzymatic activity of a NM is known, agents can be screened for their ability to decrease or increase the activity or amount of activity present in a cell.

Expression can be monitored according to any convenient method. Protein or mRNA can be monitored. Any technique known in the art for monitoring specific genes' expression can be used, including but not limited to ELISAs, SAGE, custom or commercial microarray hybridization, Western blots. Changes in expression of a single marker may be used as a criterion for significant effect as a potential pro-neuronal cell death or anti-cell death agent. However, it also may be desirable to screen for test substances which are able to modulate the expression of groups of such markers, such as modulators of at least 5, 10, 15, or 20 of the relevant markers. Inhibition of NM protein activity can also be used as a drug screen.

Neuronal cell death markers identified herein were identified using available reagents for probes. In some cases these probes are human. In other case they derive from other mammalian species. Each gene has an ortholog in humans, and the human ortholog is to be used for treating humans. When cells, cell lines, and whole animal models of other species are used, it is preferred that the corresponding ortholog be used. Nonetheless, as demonstrated in the examples below, probes for orthologs of other species can be used.

Test substances for screening can come from any source. They can be from libraries of natural products, combinatorial chemical libraries, biological products made by recombinant libraries, etc. The source of the test substances is not critical to the invention. The present invention provides means for screening compounds and compositions which may previously have been overlooked in other screening schemes.

Nucleic acids and the corresponding encoded proteins of the markers of the present invention can be used therapeutically in a variety of modes. The nucleic acids and encoded proteins can be administered by any means known in the art. Such methods include, using liposomes, nanospheres, viral vectors, non-viral vectors comprising polycations, etc. Suitable viral vectors include adenovirus, retroviruses, and sindbis virus. Administration modes can be any known in the art, including parenteral, intravenous, intramuscular, intraperitoneal, topical, intranasal, intrarectal, intrabronchial, etc. Such administrations can be used to reduce or eliminate cell death (down-regulated genes or proteins) or induce cell death (up-regulated genes or proteins). The pathological condition of the patient will determine which type of gene or protein should be used.

Specific biological antagonists of NMs can also be used to therapeutic benefit. For example, antibodies, T cells specific for an NM, antisense to an NM, and ribozymes specific for an NM can be used to restrict, inhibit, reduce, and/or diminish neuronal cell death (up-regulated genes or proteins). Conversely, antagonists of down-regulated genes or proteins can be used to induce or stimulated neuronal cell death. Such antagonists can be administered as is known in the art for these classes of antagonists generally.

Mouse counterparts to human NMs can be used in mouse models or in cell lines or in vitro to evaluate potential anti-neuronal cell death or pro-neuronal cell death compounds or therapies. Their expression can be monitored as an indication of effect.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

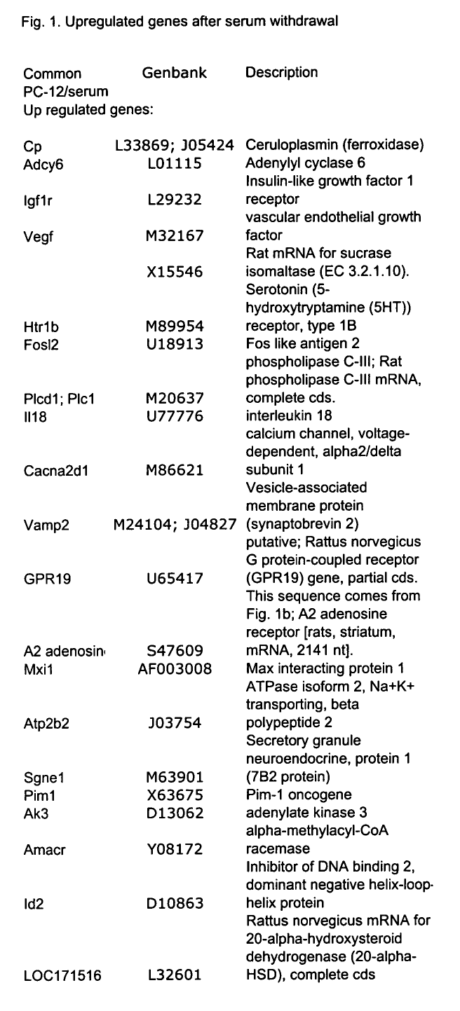

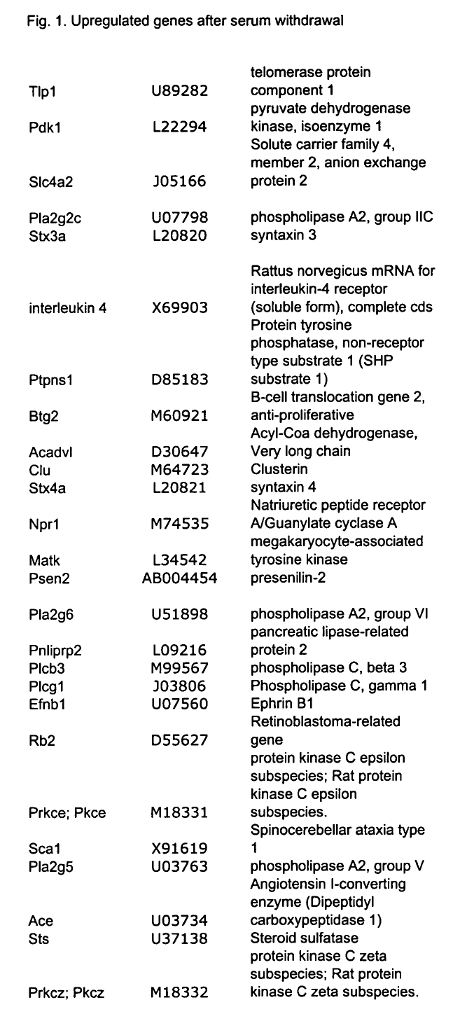

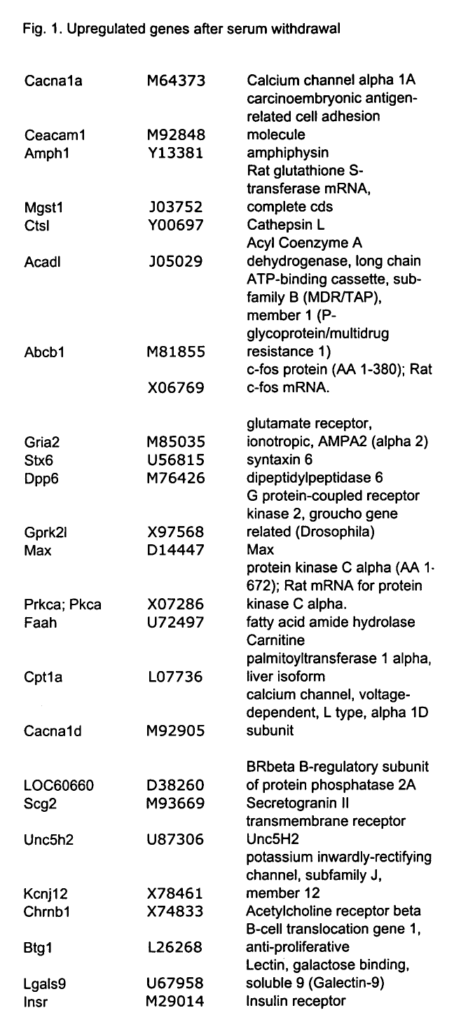

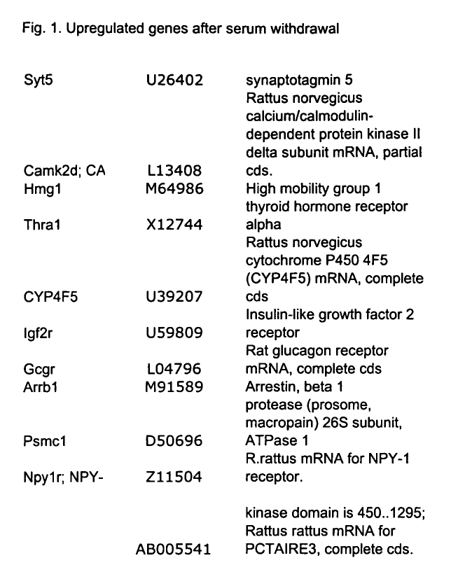

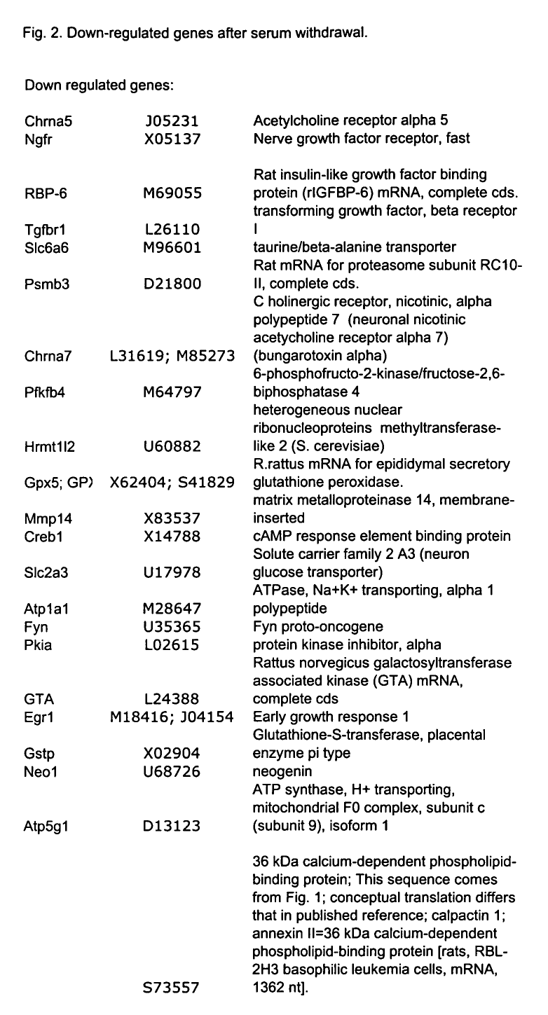

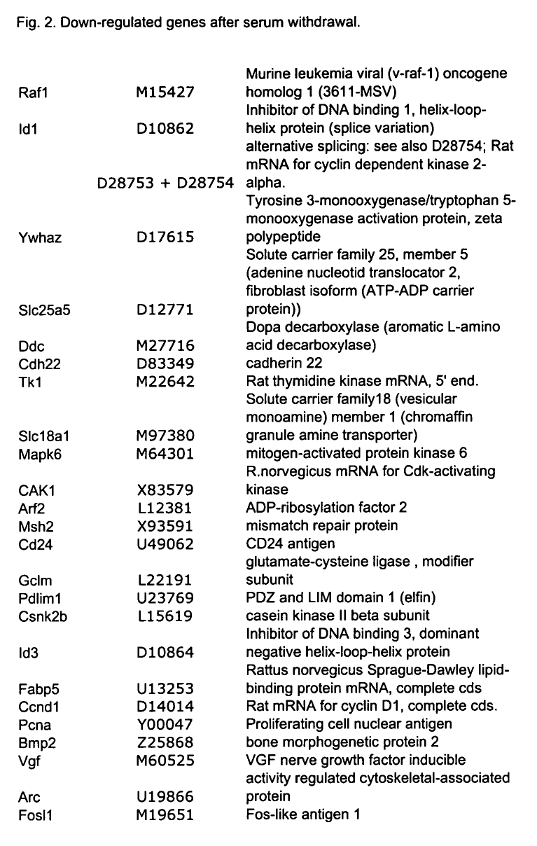

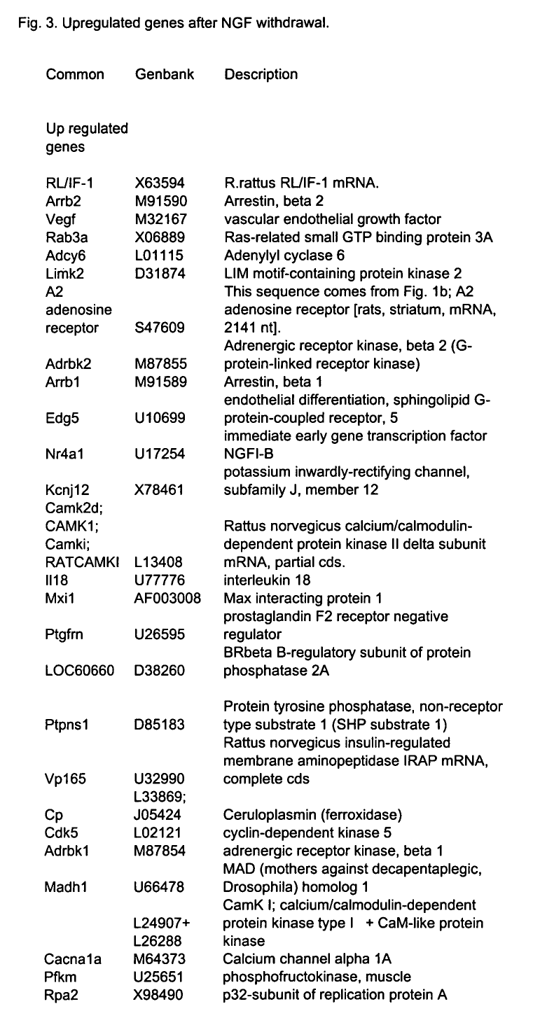

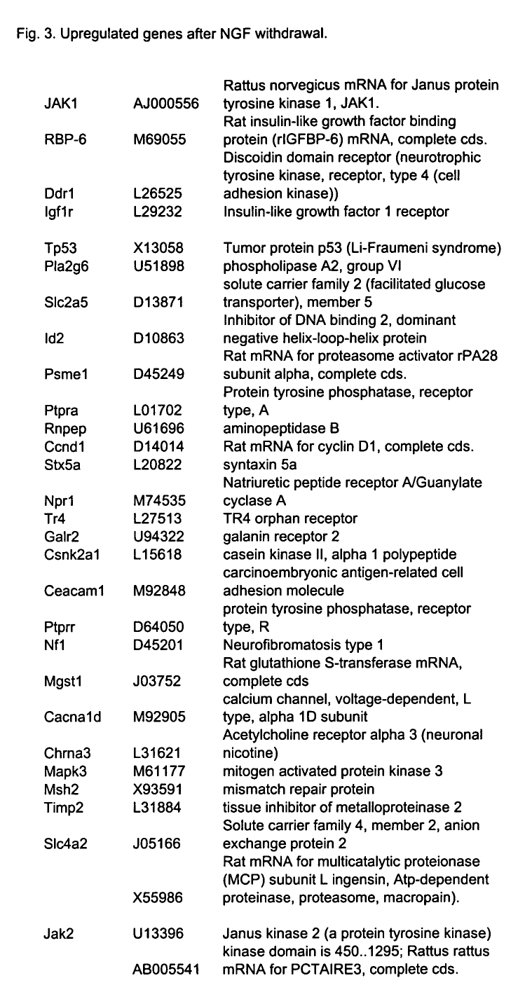

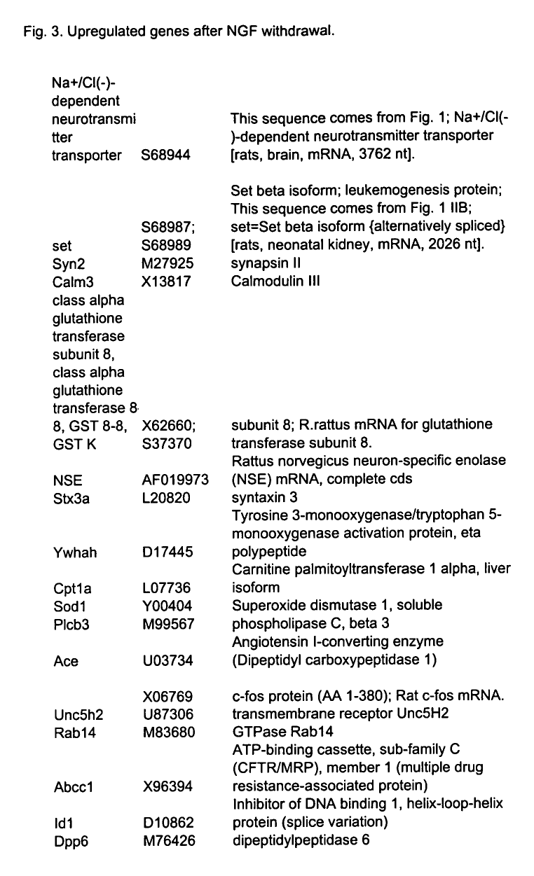

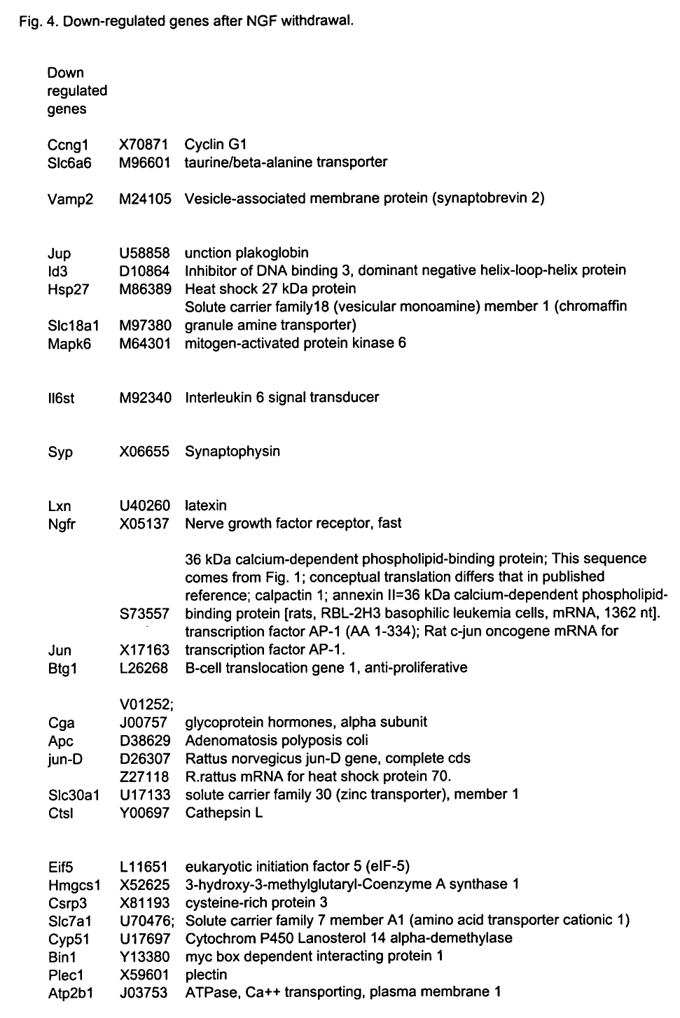

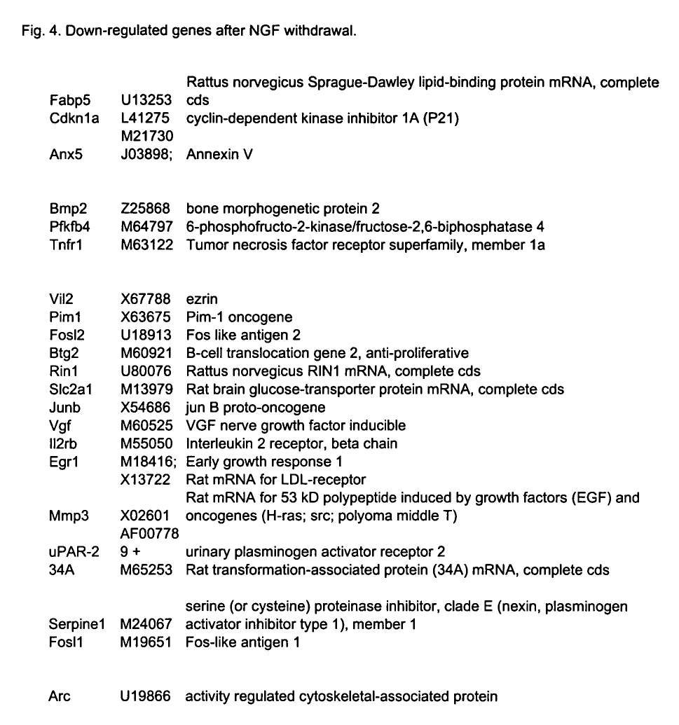

1. A method of inhibiting neuronal cell death, comprising:
administering to a subject in need thereof an effective amount of an isolated molecule comprising an antibody variable region which specifically binds to a neuronal marker (NM) protein selected from the group consisting of: Ceruloplasmin (ferroxidase); Adenylyl cyclase 6; Insulin-like growth factor 1 receptor; vascular endothelial growth factor; Rat mRNA for sucrase isomaltase (EC 3.2.1.10).; Serotonin (5-hydroxytryptamine (5HT)) receptor, type 1B; Fos like antigen 2; phospholipase C-III; Rat phospholipase C-III mRNA, complete cds.; interleukin 18; calcium channel, voltage-dependent, alpha2/delta subunit 1; Vesicle-associated membrane protein (synaptobrevin 2); putative; Rattus norvegicus G protein-coupled receptor (GPR19) gene, partial cds.; This sequence comes from FIG. 1b; A2 adenosine receptor [rats, striatum, mRNA, 2141 nt].; Max interacting protein 1; ATPase isoform 2, Na+K+ transporting, beta polypeptide 2; Secretory granule neuroendocrine, protein 1 (7B2 protein); Pim-1 oncogene; adenylate kinase 3; alpha-methylacyl-CoA racemase; Inhibitor of DNA binding 2, dominant negative helix-loop-helix protein; Rattus norvegicus mRNA for 20-alpha-hydroxysteroid dehydrogenase (20-alpha-HSD), complete cds; telomerase protein component 1; pyruvate dehydrogenase kinase, isoenzyme 1; Solute carrier family 4, member 2, anion exchange protein 2; phospholipase A2, group IIC; syntaxin 3; Rattus norvegicus mRNA for interleukin-4 receptor (soluble form), complete cds; Protein tyrosine phosphatase, non-receptor type substrate 1 (SHP substrate 1); B-cell translocation gene 2, anti-proliferative; Acyl-Coa dehydrogenase, Very long chain; Clusterin; syntaxin 4; Natriuretic peptide receptor A/Guanylate cyclase A; megakaryocyte-associated tyrosine kinase; presenilin-2; phospholipase A2, group VI; pancreatic lipase-related protein 2; phospholipase C, beta 3; Phospholipase C, gamma 1; Ephrin B1; Retinoblastoma-related gene; protein kinase C epsilon subspecies; Rat protein kinase C epsilon subspecies.; Spinocerebellar ataxia type 1; phospholipase A2, group V; Angiotensin I-converting enzyme (Dipeptidyl carboxypeptidase 1); Steroid sulfatase; protein kinase C zeta subspecies; Rat protein kinase C zeta subspecies.; Calcium channel alpha 1A; carcinoembryonic antigen-related cell adhesion molecule; amphiphysin; Rat glutathione S-transferase mRNA, complete cds; Cathepsin L; Acyl Coenzyme A dehydrogenase, long chain; ATP-binding cassette, sub-family B (MDR/TAP), member 1 (P-glycoprotein/multidrug resistance 1); c-fos protein (AA 1-380); Rat c-fos mRNA.; glutamate receptor, ionotropic, AMPA2 (alpha 2); syntaxin 6; dipeptidylpeptidase 6; G protein-coupled receptor kinase 2, groucho gene related (Drosophila); Max; protein kinase C alpha (AA 1-672); Rat mRNA for protein kinase C alpha.; fatty acid amide hydrolase; Carnitine palmitoyltransferase 1 alpha, liver isoform; calcium channel, voltage-dependent, L type, alpha 1D subunit; BRbeta B-regulatory subunit of protein phosphatase 2A; Secretogranin II; transmembrane receptor Unc5H2; potassium inwardly-rectifying channel, subfamily J, member 12; Acetylcholine receptor beta; B-cell translocation gene 1, anti-proliferative; Lectin, galactose binding, soluble 9 (Galectin-9); Insulin receptor; synaptotagmin 5; Rattus norvegicus calcium/calmodulin-dependent protein kinase II delta subunit mRNA, partial cds.; High mobility group 1; thyroid hormone receptor alpha; Rattus norvegicus cytochrome P450 4F5 (CYP4F5) mRNA, complete cds; Insulin-like growth factor 2 receptor; Rat glucagon receptor mRNA, complete cds; Arrestin, beta 1; protease (prosome, macropain) 26S subunit, ATPase 1; R. rattus mRNA for NPY-1 receptor.; kinase domain is 450..1295; Rattus rattus mRNA for PCTAIRE3, complete cds.; R. rattus RL/IF-1 mRNA.; Arrestin, beta 2; vascular endothelial growth factor; Ras-related small GTP binding protein 3A; Adenylyl cyclase 6; LIM motif-containing protein kinase 2; This sequence comes from FIG. 1b; A2 adenosine receptor [rats, striatum, mRNA, 2141 nt].; Adrenergic receptor kinase, beta 2 (G-protein-linked receptor kinase); Arrestin, beta 1; endothelial differentiation, sphingolipid G-protein-coupled receptor, 5; immediate early gene transcription factor NGFI-B; potassium inwardly-rectifying channel, subfamily J, member 12; Rattus norvegicus calcium/calmodulin-dependent protein kinase II delta subunit mRNA, partial cds.; interleukin 18; Max interacting protein 1; prostaglandin F2 receptor negative regulator; BRbeta B-regulatory subunit of protein phosphatase 2A; Protein tyrosine phosphatase, non-receptor type substrate 1 (SHP substrate 1); Rattus norvegicus insulin-regulated membrane aminopeptidase IRAP mRNA, complete cds; Ceruloplasmin (ferroxidase); cyclin-dependent kinase 5; adrenergic receptor kinase, beta 1; MAD (mothers against decapentaplegic, Drosophila) homolog 1; CamK I; calcium/calmodulin-dependent protein kinase type I +CaM-like protein kinase; Calcium channel alpha 1A; phosphofructokinase, muscle; p32-subunit of replication protein A; Rattus norvegicus mRNA for Janus protein tyrosine kinase 1, JAK1.; Rat insulin-like growth factor binding protein (rIGFBP-6) mRNA, complete cds.; Discoidin domain receptor (neurotrophic tyrosine kinase, receptor, type 4 (cell adhesion kinase)); Insulin-like growth factor 1 receptor; Tumor protein p53 (Li-Fraumeni syndrome); phospholipase A2, group VI; solute carrier family 2 (facilitated glucose transporter), member 5; Inhibitor of DNA binding 2, dominant negative helix-loop-helix protein; Rat mRNA for proteasome activator rPA28 subunit alpha, complete cds.; Protein tyrosine phosphatase, receptor type, A; aminopeptidase B; Rat mRNA for cyclin D1, complete cds.; syntaxin Sa; Natriuretic peptide receptor A/Guanylate cyclase A; TR4 orphan receptor; galanin receptor 2; casein kinase II, alpha 1 polypeptide; carcinoembryonic antigen-related cell adhesion molecule; protein tyrosine phosphatase, receptor type, R; Neurofibromatosis type 1; Rat glutathione S-transferase mRNA, complete cds; calcium channel, voltage-dependent, L type, alpha 1D subunit; Acetylcholine receptor alpha 3 (neuronal nicotine); mitogen activated protein kinase 3; mismatch repair protein; tissue inhibitor of metalloproteinase 2; Solute carrier family 4, member 2, anion exchange protein 2; Rat mRNA for multicatalytic proteionase (MCP) subunit L ingensin, Atp-dependent proteinase, proteasome, macropain).; Janus kinase 2 (a protein tyrosine kinase); kinase domain is 450..1295; Rattus rattus mRNA for PCTAIRE3, complete cds.; This sequence comes from FIG. 1; Na+/Cl(−)-dependent neurotransmitter transporter [rats, brain, mRNA, 3762 nt].; Set beta isoform; leukemogenesis protein; This sequence comes from FIG. 1 IIB; set=Set beta isoform {alternatively spliced} [rats, neonatal kidney, mRNA, 2026 nt].; synapsin II; Calmodulin III; subunit 8; R. rattus mRNA for glutathione transferase subunit 8.; Rattus norvegicus neuron-specific enolase (NSE) mRNA, complete cds; syntaxin 3; Tyrosine 3monooxygenase/tryptophan 5-monooxygenase activation protein, eta polypeptide; Carnitine palmitoyltransferase 1 alpha, liver isoform; Superoxide dismutase 1, soluble; phospholipase C, beta 3; Angiotensin I-converting enzyme (Dipeptidyl carboxypeptidase 1); c-fos protein (AA 1-380); Rat c-fos mRNA.; transmembrane receptor Unc5H2; GTPase Rab14; ATP-binding cassette, sub-family C (CFTR/MRP), member 1 (multiple drug resistance-associated protein); Inhibitor of DNA binding 1, helix-loop-helix protein (splice variation); and dipeptidylpeptidase 6, whereby neuronal cell death is inhibited.

2. The method of claim 1 wherein the subject has retinal cell degeneration.

3. The method of claim 1 wherein the subject has Alzheimer's disease.

4. The method of claim 1 wherein the subject has diabetic retinopathy.

5. The method of claim 1 wherein the subject has Huntington's disease.

6. The method of claim 1 wherein the subject has spinal cord injury.

7. The method of claim 1 wherein the subject has Parkinson's disease.

8. The method of claim 1 wherein the subject has glaucoma.

9. The method of claim 1 wherein the subject has age-related macular degeneration.

10. A method of preventing neuronal cell death in a mammal, comprising:
administering to the mammal a nucleic acid molecule comprising a coding sequence for a neuronal marker (NM) protein selected from the group consisting of: NM Acetylcholine receptor alpha 5; Nerve growth factor receptor, fast; Rat insulin-like growth factor binding protein (rIGFBP-6) mRNA, complete cds.; transforming growth factor, beta receptor I; taurine/beta-alanine transporter; Rat mRNA for proteasome subunit RC10-11, complete cds.; C holinergic receptor, nicotinic, alpha polypeptide 7 (neuronal nicotinic acetycholine receptor alpha 7) (bungarotoxin alpha); 6-phosphofructo-2-kinase/fructose-2,6-biphosphatase 4; heterogeneous nuclear ribonucleoproteins methyltransferase-like 2 (S. cerevisiae); R. rattus mRNA for epididymal secretory glutathione peroxidase.; matrix metalloproteinase 14, membrane-inserted; cAMP response element binding protein; Solute carrier family 2 A3 (neuron glucose transporter); ATPase, Na+K+ transporting, alpha 1 polypeptide; Fyn proto-oncogene; protein kinase inhibitor, alpha; Rattus norvegicus galactosyltransferase associated kinase (GTA) mRNA, complete cds; Early growth response 1; Glutathione-S-transferase, placental enzyme pi type; neogenin; ATP synthase, H+ transporting, mitochondrial F0 complex, subunit c (subunit 9), isoform 1; 36 kDa calcium-dependent phospholipid-binding protein; This sequence comes from FIG. 1; conceptual translation differs that in published reference; calpactin 1; annexin II=36 kDa calcium-dependent phospholipid-binding protein [rats, RBL-2H3 basophilic leukemia cells, mRNA, 1362 nt].; Murine leukemia viral (v-raf-1) oncogene homolog 1 (3611-MSV); Inhibitor of DNA binding 1, helix-loop-helix protein (splice variation); alternative splicing: see also D28754; Rat mRNA for cyclin dependent kinase 2-alpha.; Tyrosine 3-monooxygenase/tryptophan 5-monooxygenase activation protein, zeta polypeptide; Solute carrier family 25, member 5 (adenine nucleotid translocator 2, fibroblast isoform (ATP-ADP carrier protein)); Dopa decarboxylase (aromatic L-amino acid decarboxylase); cadherin 22; Rat thymidine kinase mRNA, 5′ end.; Solute carrier family18 (vesicular monoamine) member 1 (chromaffin granule amine transporter); mitogen-activated protein kinase 6; R. norvegicus mRNA for Cdk-activating kinase; ADP-ribosylation factor 2; mismatch repair protein; CD24 antigen; glutamate-cysteine ligase, modifier subunit; PDZ and LIM domain 1 (elfin); casein kinase II beta subunit; Inhibitor of DNA binding 3, dominant negative helix-loop-helix protein; Rattus norvegicus Sprague-Dawley lipid-binding protein mRNA, complete cds; Rat mRNA for cyclin D1, complete cds.; Proliferating cell nuclear antigen; bone morphogenetic protein 2; VGF nerve growth factor inducible; activity regulated cytoskeletal-associated protein; Fos-like antigen 1; Cyclin G1; taurine/beta-alanine transporter; Vesicle-associated membrane protein (synaptobrevin 2); unction plakoglobin; Inhibitor of DNA binding 3, dominant negative helix-loop-helix protein; Heat shock 27 kDa protein; Solute carrier family18 (vesicular monoamine) member 1 (chromaffin granule amine transporter); mitogen-activated protein kinase 6; Interleukin 6 signal transducer; Synaptophysin; latexin; Nerve growth factor receptor, fast; 36 kDa calcium-dependent phospholipid-binding protein; This sequence comes from FIG. 1; conceptual translation differs that in published reference; calpactin 1; annexin II=36 kDa calcium-dependent phospholipid-binding protein [rats, RBL-2H3 basophilic leukemia cells, mRNA, 1362 nt].; transcription factor AP-1 (AA 1-334); Rat c-jun oncogene mRNA for transcription factor AP-1.; B-cell translocation gene 1, anti-proliferativeputative anti-proliferative factor; glycoprotein hormones, alpha subunit; Adenomatosis polyposis coli; Rattus norvegicus jun-D gene, complete cds; R. rattus mRNA for heat shock protein 70.; solute carrier family 30 (zinc transporter), member 1zinc transporter; Cathepsin L; eukaryotic initiation factor 5 (eIF-5); 3-hydroxy-3methylglutaryl-Coenzyme A synthase 1; cysteine-rich protein 3; Solute carrier family 7 member A1 (amino acid transporter cationic 1); Cytochrom P450 Lanosterol 14 alpha-demethylase; myc box dependent interacting protein 1; plectin; ATPase, Ca++ transporting, plasma membrane 1; Rattus norvegicus Sprague-Dawley lipid-binding protein mRNA, complete cds; cyclin-dependent kinase inhibitor 1A (P21); Annexin V; bone morphogenetic protein 2; 6-phosphofructo-2kinase/fructose-2,6-biphosphatase 4; Tumor necrosis factor receptor superfamily, member 1a; ezrin; Pim-1 oncogene; Fos like antigen 2transcription factor; B-cell translocation gene 2, anti-proliferative; Rattus norvegicus RIN1 mRNA, complete cds; Rat brain glucose-transporter protein mRNA, complete cds; jun B proto-oncogene; VGF nerve growth factor inducible; Interleukin 2 receptor, beta chain; Early growth response 1; Rat mRNA for LDL-receptor; Rat mRNA for 53 kD polypeptide induced by growth factors (EGF) and oncogenes (H-ras; src; polyoma middle T); urinary plasminogen activator receptor 2urinary-type plasminogen activator receptor; Rat transformation-associated protein (34A) mRNA, complete cds; serine (or cysteine) proteinase inhibitor, clade E (nexin, plasminogen activator inhibitor type 1), member 1; Fos-like antigen 1; and activity regulated cytoskeletal-associated protein, whereby neuronal cell death in the mammal is inhibited or prevented.

11. A method of preventing neuronal cell death in a mammal, comprising:
administering to the mammal a purified human neuronal marker (NM) protein selected from the group consisting of: NM Acetylcholine receptor alpha 5; Nerve growth factor receptor, fast; Rat insulin-like growth factor binding protein (rIGFBP-6) mRNA, complete cds.; transforming growth factor, beta receptor I; taurine/beta-alanine transporter; Rat mRNA for proteasome subunit RC10-II, complete cds.; C holinergic receptor, nicotinic, alpha polypeptide 7 (neuronal nicotinic acetycholine receptor alpha 7) (bungarotoxin alpha); 6-phosphofructo-2kinase/fructose-2,6-biphosphatase 4; heterogeneous nuclear ribonucleoproteins methyltransferase-like 2 (S. cerevisiae); R. rattus mRNA for epididymal secretory glutathione peroxidase.; matrix metalloproteinase 14, membrane-inserted; cAMP response element binding protein; Solute carrier family 2 A3 (neuron glucose transporter); ATPase, Na+K+ transporting, alpha 1 polypeptide; Fyn proto-oncogene; protein kinase inhibitor, alpha; Rattus norvegicus galactosyltransferase associated kinase (GTA) mRNA, complete cds; Early growth response 1; Glutathione-S-transferase, placental enzyme pi type; neogenin; ATP synthase, H+ transporting, mitochondrial F0 complex, subunit c (subunit 9), isoform 1; 36 kDa calcium-dependent phospholipid-binding protein; This sequence comes from FIG. 1; conceptual translation differs that in published reference; calpactin 1; annexin II=36 kDa calcium-dependent phospholipid-binding protein [rats, RBL-2H3 basophilic leukemia cells, mRNA, 1362 nt].; Murine leukemia viral (v-raf-1) oncogene homolog 1 (3611-MSV); Inhibitor of DNA binding 1, helix-loop-helix protein (splice variation); alternative splicing: see also D28754; Rat mRNA for cyclin dependent kinase 2-alpha.; Tyrosine 3-monooxygenase/tryptophan 5-monooxygenase activation protein, zeta polypeptide; Solute carrier family 25, member 5 (adenine nucleotid translocator 2, fibroblast isoform (ATP-ADP carrier protein)); Dopa decarboxylase (aromatic L-amino acid decarboxylase); cadherin 22; Rat thymidine kinase mRNA, 5′ end.; Solute carrier family18 (vesicular monoamine) member 1 (chromaffin granule amine transporter); mitogen-activated protein kinase 6; R. norvegicus mRNA for Cdk-activating kinase; ADP-ribosylation factor 2; mismatch repair protein; CD24 antigen; glutamate-cysteine ligase, modifier subunit; PDZ and LIM domain 1 (elfin); casein kinase II beta subunit; Inhibitor of DNA binding 3, dominant negative helix-loop-helix protein; Rattus norvegicus Sprague-Dawley lipid-binding protein mRNA, complete cds; Rat mRNA for cyclin D1, complete cds.; Proliferating cell nuclear antigen; bone morphogenetic protein 2; VGF nerve growth factor inducible; activity regulated cytoskeletal-associated protein; Fos-like antigen 1; Cyclin G1; taurine/beta-alanine transporter; Vesicle-associated membrane protein (synaptobrevin 2); unction plakoglobin; Inhibitor of DNA binding 3, dominant negative helix-loop-helix protein; Heat shock 27 kDa protein; Solute carrier family18 (vesicular monoamine) member 1 (chromaffin granule amine transporter); mitogen-activated protein kinase 6; Interleukin 6 signal transducer; Synaptophysin; latexin; Nerve growth factor receptor, fast; 36 kDa calcium-dependent phospholipid-binding protein; This sequence comes from FIG. 1; conceptual translation differs that in published reference; calpactin 1; annexin II=36 kDa calcium-dependent phospholipid-binding protein [rats, RBL-2H3 basophilic leukemia cells, mRNA, 1362 nt].; transcription factor AP-1 (AA 1-334); Rat c-jun oncogene mRNA for transcription factor AP-1.; B-cell translocation gene 1, anti-proliferativeputative anti-proliferative factor; glycoprotein hormones, alpha subunit; Adenomatosis polyposis coli; Rattus norvegicus jun-D gene, complete cds; R. rattus mRNA for heat shock protein 70.; solute carrier family 30 (zinc transporter), member 1 zinc transporter; Cathepsin L; eukaryotic initiation factor 5 (eIF-5); 3-hydroxy-3-methylglutaryl-Coenzyme A synthase 1; cysteine-rich protein 3; Solute carrier family 7 member A1 (amino acid transporter cationic 1); Cytochrom P450 Lanosterol 14 alpha-demethylase; myc box dependent interacting protein 1; plectin; ATPase, Ca++ transporting, plasma membrane 1; Rattus norvegicus Sprague-Dawley lipid-binding protein mRNA, complete cds; cyclin-dependent kinase inhibitor 1A (P21); Annexin V; bone morphogenetic protein 2; 6phosphofructo-2-kinase/fructose-2,6-biphosphatase 4; Tumor necrosis factor receptor superfamily, member 1a; ezrin; Pim-1 oncogene; Fos like antigen 2transcription factor; B-cell translocation gene 2, anti-proliferative; Rattus norvegicus RIN1 mRNA, complete cds; Rat brain glucose-transporter protein mRNA, complete cds; jun B proto-oncogene; VGF nerve growth factor inducible; Interleukin 2 receptor, beta chain; Early growth response 1; Rat mRNA for LDL-receptor; Rat mRNA for 53 kD polypeptide induced by growth factors (EGF) and oncogenes (H-ras; src; polyoma middle T); urinary plasminogen activator receptor 2urinary-type plasminogen activator receptor; Rat transformation-associated protein (34A) mRNA, complete cds; serine (or cysteine) proteinase inhibitor, clade E (nexin, plasminogen activator inhibitor type 1), member 1; Fos-like antigen 1; and activity regulated cytoskeletal-associated protein, whereby neuronal cell death in the mammal is inhibited or prevented.

12. The method of claim 10 or 11 wherein the subject has retinal cell degeneration.

13. The method of claim 10 or 11 wherein the subject has Alzheimer's disease.

14. The method of claim 10 or 11 wherein the subject has diabetic retinopathy.

15. The method of claim 10 or 11 wherein the subject has Huntington's disease.

16. The method of claim 10 or 11 wherein the subject has spinal cord injury.

17. The method of claim 10 or 11 wherein the subject has Parkinson's disease.

18. The method of claim 10 or 11 wherein the subject has glaucoma.

19. The method of claim 10 or 11 wherein the subject has age-related macular degeneration.

20. A method of identifying regions of neuronal cell death in a patient, comprising:
administering to a patient a molecule comprising an antibody variable region which specifically binds to a neuronal marker (NM) protein selected from the group consisting of: Ceruloplasmin (ferroxidase); Adenylyl cyclase 6; Insulin-like growth factor 1 receptor; vascular endothelial growth factor; Rat mRNA for sucrase isomaltase (EC 3.2.1.10).; Serotonin (5hydroxytryptamine (5HT)) receptor, type 1B; Fos like antigen 2; phospholipase C-III; Rat phospholipase C-III mRNA, complete cds.; interleukin 18; calcium channel, voltage-dependent, alpha2/delta subunit 1; Vesicle-associated membrane protein (synaptobrevin 2); putative; Rattus norvegicus G protein-coupled receptor (GPR19) gene, partial cds.; This sequence comes from FIG. 1b; A2 adenosine receptor [rats, striatum, mRNA, 2141 nt].; Max interacting protein 1; ATPase isoform 2, Na+K+ transporting, beta polypeptide 2; Secretory granule neuroendocrine, protein 1 (7B2 protein); Pim-1 oncogene; adenylate kinase 3; alpha-methylacyl-CoA racemase; Inhibitor of DNA binding 2, dominant negative helix-loop-helix protein; Rattus norvegicus mRNA for 20-alpha-hydroxysteroid dehydrogenase (20-alpha-HSD), complete cds; telomerase protein component 1; pyruvate dehydrogenase kinase, isoenzyme 1; Solute carrier family 4, member 2, anion exchange protein 2; phospholipase A2, group IIC; syntaxin 3; Rattus norvegicus mRNA for interleukin-4 receptor (soluble form), complete cds; Protein tyrosine phosphatase, non-receptor type substrate 1 (SHP substrate 1); B-cell translocation gene 2, anti-proliferative; Acyl-Coa dehydrogenase, Very long chain; Clusterin; syntaxin 4; Natriuretic peptide receptor A/Guanylate cyclase A; megakaryocyte-associated tyrosine kinase; presenilin-2; phospholipase A2, group VI; pancreatic lipase-related protein 2; phospholipase C, beta 3; Phospholipase C, gamma 1; Ephrin B1; Retinoblastoma-related gene; protein kinase C epsilon subspecies; Rat protein kinase C epsilon subspecies.; Spinocerebellar ataxia type 1; phospholipase A2, group V; Angiotensin I-converting enzyme (Dipeptidyl carboxypeptidase 1); Steroid sulfatase; protein kinase C zeta subspecies; Rat protein kinase C zeta subspecies.; Calcium channel alpha 1A; carcinoembryonic antigen-related cell adhesion molecule; amphiphysin; Rat glutathione S-transferase mRNA, complete cds; Cathepsin L; Acyl Coenzyme A dehydrogenase, long chain; ATP-binding cassette, sub-family B (MDR/TAP), member 1 (P-glycoprotein/multidrug resistance 1); c-fos protein (AA 1-380); Rat c-fos mRNA.; glutamate receptor, ionotropic, AMPA2 (alpha 2); syntaxin 6; dipeptidylpeptidase 6; G protein-coupled receptor kinase 2, groucho gene related (Drosophila); Max; protein kinase C alpha (AA 1-672); Rat mRNA for protein kinase C alpha.; fatty acid amide hydrolase; Carnitine palmitoyltransferase 1 alpha, liver isoform; calcium channel, voltage-dependent, L type, alpha 1D subunit; BRbeta B-regulatory subunit of protein phosphatase 2A; Secretogranin II; transmembrane receptor Unc5H2; potassium inwardly-rectifying channel, subfamily J, member 12; Acetylcholine receptor beta; B-cell translocation gene 1, anti-proliferative; Lectin, galactose binding, soluble 9 (Galectin-9); Insulin receptor; synaptotagmin 5; Rattus norvegicus calcium/calmodulin-dependent protein kinase II delta subunit mRNA, partial cds.; High mobility group 1; thyroid hormone receptor alpha; Rattus norvegicus cytochrome P450 4F5 (CYP4F5) mRNA, complete cds; Insulin-like growth factor 2 receptor; Rat glucagon receptor mRNA, complete cds; Arrestin, beta 1; protease (prosome, macropain) 26S subunit, ATPase 1; R. rattus mRNA for NPY-1 receptor.; kinase domain is 450..1295; Rattus rattus mRNA for PCTAIRE3, complete cds.; R. rattus RL/IF-1 mRNA.; Arrestin, beta 2; vascular endothelial growth factor; Ras-related small GTP binding protein 3A; Adenylyl cyclase 6; LIM motif-containing protein kinase 2; This sequence comes from FIG. 1b; A2 adenosine receptor [rats, striatum, mRNA, 2141 nt].; Adrenergic receptor kinase, beta 2 (G-protein-linked receptor kinase); Arrestin, beta 1; endothelial differentiation, sphingolipid G-protein-coupled receptor, 5; immediate early gene transcription factor NGFI-B; potassium inwardly-rectifying channel, subfamily J, member 12; Rattus norvegicus calcium/calmodulin-dependent protein kinase II delta subunit mRNA, partial cds.; interleukin 18; Max interacting protein 1; prostaglandin F2 receptor negative regulator; BRbeta B-regulatory subunit of protein phosphatase 2A; Protein tyrosine phosphatase, non-receptor type substrate 1 (SHP substrate 1); Rattus norvegicus insulin-regulated membrane aminopeptidase IRAP mRNA, complete cds; Ceruloplasmin (ferroxidase); cyclin-dependent kinase 5; adrenergic receptor kinase, beta 1; MAD (mothers against decapentaplegic, Drosophila) homolog 1; CamK I; calcium/calmodulin-dependent protein kinase type I +CaM-like protein kinase; Calcium channel alpha 1A; phosphofructokinase, muscle; p32-subunit of replication protein A; Rattus norvegicus mRNA for Janus protein tyrosine kinase 1, JAK1.; Rat insulin-like growth factor binding protein (rIGFBP-6) mRNA, complete cds.; Discoidin domain receptor (neurotrophic tyrosine kinase, receptor, type 4 (cell adhesion kinase)); Insulin-like growth factor 1 receptor; Tumor protein p53 (Li-Fraumeni syndrome); phospholipase A2; group VI; solute carrier family 2 (facilitated glucose transporter), member 5; Inhibitor of DNA binding 2, dominant negative helix-loop-helix protein; Rat mRNA for proteasome activator rPA28 subunit alpha, complete cds.; Protein tyrosine phosphatase, receptor type, A; aminopeptidase B; Rat mRNA for cyclin D1, complete cds.; syntaxin 5a; Natriuretic peptide receptor A/Guanylate cyclase A; TR4 orphan receptor; galanin receptor 2; casein kinase II, alpha 1 polypeptide; carcinoembryonic antigen-related cell adhesion molecule; protein tyrosine phosphatase, receptor type, R; Neurofibromatosis type 1; Rat glutathione S-transferase mRNA, complete cds; calcium channel, voltage-dependent, L type, alpha 1D subunit; Acetylcholine receptor alpha 3 (neuronal nicotine); mitogen activated protein kinase 3; mismatch repair protein; tissue inhibitor of metalloproteinase 2; Solute carrier family 4, member 2, anion exchange protein 2; Rat mRNA for multicatalytic proteionase (MCP) subunit L ingensin, Atp-dependent proteinase, proteasome, macropain).; Janus kinase 2 (a protein tyrosine kinase); kinase domain is 450..1295; Rattus rattus mRNA for PCTAIRE3, complete cds.; This sequence comes from FIG. 1; Na+/Cl(−)-dependent neurotransmitter transporter [rats, brain, mRNA, 3762 nt].; Set beta isoform; leukemogenesis protein; This sequence comes from FIG. 1 IIB; set=Set beta isoform {alternatively spliced} [rats, neonatal kidney, mRNA, 2026 nt].; synapsin II; Calmodulin III; subunit 8; R. rattus mRNA for glutathione transferase subunit 8.; Rattus norvegicus neuron-specific enolase (NSE) mRNA, complete cds; syntaxin 3; Tyrosine 3monooxygenase/tryptophan 5-monooxygenase activation protein, eta polypeptide; Carnitine palmitoyltransferase 1 alpha, liver isoform; Superoxide dismutase 1, soluble; phospholipase C, beta 3; Angiotensin I-converting enzyme (Dipeptidyl carboxypeptidase 1); c-fos protein (AA 1-380); Rat c-fos mRNA.; transmembrane receptor Unc5H2; GTPase Rab14; ATP-binding cassette, sub-family C (CFTR/MRP), member 1 (multiple drug resistance-associated protein); Inhibitor of DNA binding 1, helix-loop-helix protein (splice variation); and dipeptidylpeptidase 6, wherein the molecule is bound to a detectable moiety; and
detecting the detectable moiety in the patient, thereby identifying regions of neuronal cell death.

21. The method of claim 20 wherein the subject has retinal cell degeneration.

22. The method of claim 20 wherein the subject has Alzheimer's disease.

23. The method of claim 20 wherein the subject has diabetic retinopathy.

24. The method of claim 20 wherein the subject has Huntington's disease.

25. The method of claim 20 wherein the subject has spinal cord injury.

26. The method of claim 20 wherein the subject has Parkinson's disease.

27. The method of claim 20 wherein the subject has glaucoma.

28. The method of claim 20 wherein the subject has age-related macular degeneration.

29. A method of screening for neuronal cell death in a patient, comprising:
contacting a body fluid collected from the patient with a molecule comprising an antibody variable region which specifically binds to a neuronal marker (NM) protein selected from the group consisting of: Ceruloplasmin (ferroxidase); Adenylyl cyclase 6; Insulin-like growth factor 1 receptor; vascular endothelial growth factor; Rat mRNA for sucrase isomaltase (EC 3.2.1.10).; Serotonin (5-hydroxytryptamine (5HT)) receptor, type 1B; Fos like antigen 2; phospholipase C-III; Rat phospholipase C-III mRNA, complete cds.; interleukin 18; calcium channel, voltage-dependent, alpha2/delta subunit 1; Vesicle-associated membrane protein (synaptobrevin 2); putative; Rattus norvegicus G protein-coupled receptor (GPR19) gene, partial cds.; This sequence comes from FIG. 1b; A2 adenosine receptor [rats, striatum, mRNA, 2141 nt].; Max interacting protein 1; ATPase isoform 2, Na+K+ transporting, beta polypeptide 2; Secretory granule neuroendocrine, protein 1 (7B2 protein); Pim-1 oncogene; adenylate kinase 3; alpha-methylacyl-CoA racemase; Inhibitor of DNA binding 2, dominant negative helix-loop-helix protein; Rattus norvegicus mRNA for 20-alpha-hydroxysteroid dehydrogenase (20-alpha-HSD), complete cds; telomerase protein component 1; pyruvate dehydrogenase kinase, isoenzyme 1; Solute carrier family 4, member 2, anion exchange protein 2; phospholipase A2, group IIC; syntaxin 3; Rattus norvegicus mRNA for interleukin-4 receptor (soluble form), complete cds; Protein tyrosine phosphatase, non-receptor type substrate 1 (SHP substrate 1); B-cell translocation gene 2, anti-proliferative; Acyl-Coa dehydrogenase, Very long chain; Clusterin; syntaxin 4; Natriuretic peptide receptor A/Guanylate cyclase A; megakaryocyte-associated tyrosine kinase; presenilin-2; phospholipase A2, group VI; pancreatic lipase-related protein 2; phospholipase C, beta 3; Phospholipase C, gamma 1; Ephrin B1; Retinoblastoma-related gene; protein kinase C epsilon subspecies; Rat protein kinase C epsilon subspecies.; Spinocerebellar ataxia type 1; phospholipase A2, group V; Angiotensin I-converting enzyme (Dipeptidyl carboxypeptidase 1); Steroid sulfatase; protein kinase C zeta subspecies; Rat protein kinase C zeta subspecies.; Calcium channel alpha 1A; carcinoembryonic antigen-related cell adhesion molecule; amphiphysin; Rat glutathione S-transferase mRNA, complete cds; Cathepsin L; Acyl Coenzyme A dehydrogenase, long chain; ATP-binding cassette, sub-family B (MDR/TAP), member 1 (P-glycoprotein/multidrug resistance 1); c-fos protein (AA 1-380); Rat c-fos mRNA.; glutamate receptor, ionotropic, AMPA2 (alpha 2); syntaxin 6; dipeptidylpeptidase 6; G protein-coupled receptor kinase 2, groucho gene related (Drosophila); Max; protein kinase C alpha (AA 1-672); Rat mRNA for protein kinase C alpha.; fatty acid amide hydrolase; Carnitine palmitoyltransferase 1 alpha, liver isoform; calcium channel, voltage-dependent, L type, alpha 1D subunit; BRbeta B-regulatory subunit of protein phosphatase 2A; Secretogranin II; transmembrane receptor Unc5H2; potassium inwardly-rectifying channel, subfamily J, member 12; Acetylcholine receptor beta; B-cell translocation gene 1, anti-proliferative; Lectin, galactose binding, soluble 9 (Galectin-9); Insulin receptor; synaptotagmin 5; Rattus norvegicus calcium/calmodulin-dependent protein kinase II delta subunit mRNA, partial cds.; High mobility group 1; thyroid hormone receptor alpha; Rattus norvegicus cytochrome P450 4F5 (CYP4F5) mRNA, complete cds; Insulin-like growth factor 2 receptor; Rat glucagon receptor mRNA, complete cds; Arrestin, beta 1; protease (prosome, macropain) 26S subunit, ATPase 1; R. rattus mRNA for NPY-1 receptor.; kinase domain is 450..1295; Rattus rattus mRNA for PCTAIRE3, complete cds.; R. rattus RL/IF-1 mRNA.; Arrestin, beta 2; vascular endothelial growth factor; Ras-related small GTP binding protein 3A; Adenylyl cyclase 6; LIM motif-containing protein kinase 2; This sequence comes from FIG. 1b; A2 adenosine receptor [rats, striatum, mRNA, 2141 nt].; Adrenergic receptor kinase, beta 2 (G-protein-linked receptor kinase); Arrestin, beta 1; endothelial differentiation, sphingolipid G-protein-coupled receptor, 5; immediate early gene transcription factor NGFI-B; potassium inwardly-rectifying channel, subfamily J, member 12; Rattus norvegicus calcium/calmodulin-dependent protein kinase II delta subunit mRNA, partial cds.; interleukin 18; Max interacting protein 1; prostaglandin F2 receptor negative regulator; BRbeta B-regulatory subunit of protein phosphatase 2A; Protein tyrosine phosphatase, non-receptor type substrate 1 (SHP substrate 1); Rattus norvegicus insulin-regulated membrane aminopeptidase IRAP mRNA, complete cds; Ceruloplasmin (ferroxidase); cyclin-dependent kinase 5; adrenergic receptor kinase, beta 1; MAD (mothers against decapentaplegic, Drosophila) homolog 1; CamK I; calcium/calmodulin-dependent protein kinase type I +CaM-like protein kinase; Calcium channel alpha 1A; phosphofructokinase, muscle; p32-subunit of replication protein A; Rattus norvegicus mRNA for Janus protein tyrosine kinase 1, JAK1.; Rat insulin-like growth factor binding protein (rIGFBP-6) mRNA, complete cds.; Discoidin domain receptor (neurotrophic tyrosine kinase, receptor, type 4 (cell adhesion kinase)); Insulin-like growth factor 1 receptor; Tumor protein p53 (Li-Fraumeni syndrome); phospholipase A2, group VI; solute carrier family 2 (facilitated glucose transporter), member 5; Inhibitor of DNA binding 2, dominant negative helix-loop-helix protein; Rat mRNA for proteasome activator rPA28 subunit alpha, complete cds.; Protein tyrosine phosphatase, receptor type, A; aminopeptidase B; Rat mRNA for cyclin D1, complete cds.; syntaxin 5a; Natriuretic peptide receptor A/Guanylate cyclase A; TR4 orphan receptor; galanin receptor 2; casein kinase II, alpha 1 polypeptide; carcinoembryonic antigen-related cell adhesion molecule; protein tyrosine phosphatase, receptor type, R; Neurofibromatosis type 1; Rat glutathione S-transferase mRNA, complete cds; calcium channel, voltage-dependent, L type, alpha 1D subunit; Acetylcholine receptor alpha 3 (neuronal nicotine); mitogen activated protein kinase 3; mismatch repair protein; tissue inhibitor of metalloproteinase 2; Solute carrier family 4, member 2, anion exchange protein 2; Rat mRNA for multicatalytic proteionase (MCP) subunit L ingensin, Atp-dependent proteinase, proteasome, macropain).; Janus kinase 2 (a protein tyrosine kinase); kinase domain is 450..1295; Rattus rattus mRNA for PCTAIRE3, complete cds.; This sequence comes from FIG. 1; Na+/Cl(−)-dependent neurotransmitter transporter [rats, brain, mRNA, 3762 nt].; Set beta isoform; leukemogenesis protein; This sequence comes from FIG. 1 IIB; set=Set beta isoform {alternatively spliced} [rats, neonatal kidney, mRNA, 2026 nt].; synapsin II; Calmodulin III; subunit 8; R. rattus mRNA for glutathione transferase subunit 8.; Rattus norvegicus neuron-specific enolase (NSE) mRNA, complete cds; syntaxin 3; Tyrosine 3monooxygenase/tryptophan 5-monooxygenase activation protein, eta polypeptide; Carnitine palmitoyltransferase 1 alpha, liver isoform; Superoxide dismutase 1, soluble; phospholipase C, beta 3; Angiotensin I-converting enzyme (Dipeptidyl carboxypeptidase 1); c-fos protein (AA 1-380); Rat c-fos mRNA.; transmembrane receptor Unc5H2; GTPase Rab14; ATP-binding cassette, sub-family C (CFTR/MRP), member 1 (multiple drug resistance-associated protein); Inhibitor of DNA binding 1, helix-loop-helix protein (splice variation); and dipeptidylpeptidase 6, wherein detection of cross-reactive material in the body fluid with the molecule indicates neuronal cell death in the patient.

30. A method of promoting neuronal cell death in a patient, comprising:
administering to a patient in need of neuronal cell death a neuronal marker (NM) protein selected from the group consisting of: Ceruloplasmin (ferroxidase); Adenylyl cyclase 6; Insulin-like growth factor 1 receptor; vascular endothelial growth factor; Rat mRNA for sucrase isomaltase (EC 3.2.1.10).; Serotonin (5-hydroxytryptamine (5HT)) receptor, type 1B; Fos like antigen 2; phospholipase C-III; Rat phospholipase C-III mRNA, complete cds.; interleukin 18; calcium channel, voltage-dependent, alpha2/delta subunit 1; Vesicle-associated membrane protein (synaptobrevin 2); putative; Rattus norvegicus G protein-coupled receptor (GPR19) gene, partial cds.; This sequence comes from FIG. 1b; A2 adenosine receptor [rats, striatum, mRNA, 2141 nt].; Max interacting protein 1; ATPase isoform 2, Na+K+ transporting, beta polypeptide 2; Secretory granule neuroendocrine, protein 1 (7B2 protein); Pim-1 oncogene; adenylate kinase 3; alpha-methylacyl-CoA racemase; Inhibitor of DNA binding 2, dominant negative helix-loop-helix protein; Rattus norvegicus mRNA for 20-alpha-hydroxysteroid dehydrogenase (20-alpha-HSD), complete cds; telomerase protein component 1; pyruvate dehydrogenase kinase, isoenzyme 1; Solute carrier family 4, member 2, anion exchange protein 2; phospholipase A2, group IIC; syntaxin 3; Rattus norvegicus mRNA for interleukin-4 receptor (soluble form), complete cds; Protein tyrosine phosphatase, non-receptor type substrate 1 (SHP substrate 1); B-cell translocation gene 2, anti-proliferative; Acyl-Coa dehydrogenase, Very long chain; Clusterin; syntaxin 4; Natriuretic peptide receptor A/Guanylate cyclase A; megakaryocyte-associated tyrosine kinase; presenilin-2; phospholipase A2, group VI; pancreatic lipase-related protein 2; phospholipase C, beta 3; Phospholipase C, gamma 1; Ephrin B1; Retinoblastoma-related gene; protein kinase C epsilon subspecies; Rat protein kinase C epsilon subspecies.; Spinocerebellar ataxia type 1; phospholipase A2, group V; Angiotensin I-converting enzyme (Dipeptidyl carboxypeptidase 1); Steroid sulfatase; protein kinase C zeta subspecies; Rat protein kinase C zeta subspecies.; Calcium channel alpha 1A; carcinoembryonic antigen-related cell adhesion molecule; amphiphysin; Rat glutathione S-transferase mRNA, complete cds; Cathepsin L; Acyl Coenzyme A dehydrogenase, long chain; ATP-binding cassette, sub-family B (MDR/TAP), member 1 (P-glycoprotein/multidrug resistance 1); c-fos protein (AA 1-380); Rat c-fos mRNA.; glutamate receptor, ionotropic, AMPA2 (alpha 2); syntaxin 6; dipeptidylpeptidase 6; G protein-coupled receptor kinase 2, groucho gene related (Drosophila); Max; protein kinase C alpha (AA 1-672); Rat mRNA for protein kinase C alpha.; fatty acid amide hydrolase; Carnitine palmitoyltransferase 1 alpha, liver isoform; calcium channel, voltage-dependent, L type, alpha 1D subunit; BRbeta B-regulatory subunit of protein phosphatase 2A; Secretogranin II; transmembrane receptor Unc5H2; potassium inwardly-rectifying channel, subfamily J, member 12; Acetylcholine receptor beta; B-cell translocation gene 1, anti-proliferative; Lectin, galactose binding, soluble 9 (Galectin-9); Insulin receptor; synaptotagmin 5; Rattus norvegicus calcium/calmodulin-dependent protein kinase II delta subunit mRNA, partial cds.; High mobility group 1; thyroid hormone receptor alpha; Rattus norvegicus cytochrome P450 4F5 (CYP4F5) mRNA, complete cds; Insulin-like growth factor 2 receptor; Rat glucagon receptor mRNA, complete cds; Arrestin, beta 1; protease (prosome, macropain) 26S subunit, ATPase 1; R. rattus mRNA for NPY-1 receptor.; kinase domain is 450..1295; Rattus rattus mRNA for PCTAIRE3, complete cds.; R. rattus RL/IF-1 mRNA.; Arrestin, beta 2; vascular endothelial growth factor; Ras-related small GTP binding protein 3A; Adenylyl cyclase 6; LIM motif-containing protein kinase 2; This sequence comes from FIG. 1b; A2 adenosine receptor [rats, striatum, mRNA, 2141 nt].; Adrenergic receptor kinase, beta 2 (G-protein-linked receptor kinase); Arrestin, beta 1; endothelial differentiation, sphingolipid G-protein-coupled receptor, 5; immediate early gene transcription factor NGFI-B; potassium inwardly-rectifying channel, subfamily J, member 12; Rattus norvegicus calcium/calmodulin-dependent protein kinase II delta subunit mRNA, partial cds.; interleukin 18; Max interacting protein 1; prostaglandin F2 receptor negative regulator; BRbeta B-regulatory subunit of protein phosphatase 2A; Protein tyrosine phosphatase, non-receptor type substrate 1 (SHP substrate 1); Rattus norvegicus insulin-regulated membrane aminopeptidase IRAP mRNA, complete cds; Ceruloplasmin (ferroxidase); cyclin-dependent kinase 5; adrenergic receptor kinase, beta 1; MAD (mothers against decapentaplegic, Drosophila) homolog 1; CamK I; calcium/calmodulin-dependent protein kinase type I +CaM-like protein kinase; Calcium channel alpha 1A; phosphofructokinase, muscle; p32-subunit of replication protein A; Rattus norvegicus mRNA for Janus protein tyrosine kinase 1, JAK1.; Rat insulin-like growth factor binding protein (rIGFBP-6) mRNA, complete cds.; Discoidin domain receptor (neurotrophic tyrosine kinase, receptor, type 4 (cell adhesion kinase)); Insulin-like growth factor 1 receptor; Tumor protein p53 (Li-Fraumeni syndrome); phospholipase A2, group VI; solute carrier family 2 (facilitated glucose transporter), member 5; Inhibitor of DNA binding 2, dominant negative helix-loop-helix protein; Rat mRNA for proteasome activator rPA28 subunit alpha, complete cds.; Protein tyrosine phosphatase, receptor type, A; aminopeptidase B; Rat mRNA for cyclin D1, complete cds.; syntaxin 5a; Natriuretic peptide receptor A/Guanylate cyclase A; TR4 orphan receptor; galanin receptor 2; casein kinase II, alpha 1 polypeptide; carcinoembryonic antigen-related cell adhesion molecule; protein tyrosine phosphatase, receptor type, R; Neurofibromatosis type 1; Rat glutathione S-transferase mRNA, complete cds; calcium channel, voltage-dependent, L type, alpha 1D subunit; Acetylcholine receptor alpha 3 (neuronal nicotine); mitogen activated protein kinase 3; mismatch repair protein; tissue inhibitor of metalloproteinase 2; Solute carrier family 4, member 2, anion exchange protein 2; Rat mRNA for multicatalytic proteionase (MCP) subunit L ingensin, Atp-dependent proteinase, proteasome, macropain).; Janus kinase 2 (a protein tyrosine kinase); kinase domain is 450..1295; Rattus rattus mRNA for PCTAIRE3, complete cds.; This sequence comes from FIG. 1; Na+/Cl(−)-dependent neurotransmitter transporter [rats, brain, mRNA, 3762 nt].; Set beta isoform; leukemogenesis protein; This sequence comes from FIG. 1 IIB; set=Set beta isoform {alternatively spliced} [rats, neonatal kidney, mRNA, 2026 nt].; synapsin II; Calmodulin III; subunit 8; R. rattus mRNA for glutathione transferase subunit 8.; Rattus norvegicus neuron-specific enolase (NSE) mRNA, complete cds; syntaxin 3; Tyrosine 3monooxygenase/tryptophan 5-monooxygenase activation protein, eta polypeptide; Carnitine palmitoyltransferase 1 alpha, liver isoform; Superoxide dismutase 1, soluble; phospholipase C, beta 3; Angiotensin I-converting enzyme (Dipeptidyl carboxypeptidase 1); c-fos protein (AA 1-380); Rat c-fos mRNA.; transmembrane receptor Unc5H2; GTPase Rab14; ATP-binding cassette, sub-family C (CFTR/MRP), member 1 (multiple drug resistance-associated protein); Inhibitor of DNA binding 1, helix-loop-helix protein (splice variation); and dipeptidylpeptidase 6, whereby neuronal cell death in the patient is stimulated.

31. The method of claim 30 wherein the patient has a neuronal tumor.

32. A method of promoting neuronal cell death in a patient, comprising:
administering to a patient in need of neuronal cell death a nucleic acid molecule encoding a neuronal marker (NM) protein selected from the group consisting of Ceruloplasmin (ferroxidase); Adenylyl cyclase 6; Insulin-like growth factor 1 receptor; vascular endothelial growth factor; Rat mRNA for sucrase isomaltase (EC 3.2.1.10).; Serotonin (5-hydroxytryptamine (5HT)) receptor, type 1B; Fos like antigen 2; phospholipase C-III; Rat phospholipase c-m mRNA, complete cds.; interleukin 18; calcium channel, voltage-dependent, alpha2/delta subunit 1; Vesicle-associated membrane protein (synaptobrevin 2); putative; Rattus norvegicus G protein-coupled receptor (GPR19) gene, partial cds.; This sequence comes from FIG. 1b; A2 adenosine receptor [rats, striatum, mRNA, 2141 nt].; Max interacting protein 1; ATPase isoform 2, Na+K+ transporting, beta polypeptide 2; Secretory granule neuroendocrine, protein 1 (7B2 protein); Pim-1 oncogene; adenylate kinase 3; alpha-methylacyl-CoA racemase; Inhibitor of DNA binding 2, dominant negative helix-loop-helix protein; Rattus norvegicus mRNA for 20-alpha-hydroxysteroid dehydrogenase (20-alpha-HSD), complete cds; telomerase protein component 1; pyruvate dehydrogenase kinase, isoenzyme 1; Solute carrier family 4, member 2, anion exchange protein 2; phospholipase A2, group IIC; syntaxin 3; Rattus norvegicus mRNA for interleukin-4 receptor (soluble form), complete cds; Protein tyrosine phosphatase, non-receptor type substrate 1 (SHP substrate 1); B-cell translocation gene 2, anti-proliferative; Acyl-Coa dehydrogenase, Very long chain; Clusterin; syntaxin 4; Natriuretic peptide receptor A/Guanylate cyclase A; megakaryocyte-associated tyrosine kinase; presenilin-2; phospholipase A2, group VI; pancreatic lipase-related protein 2; phospholipase C, beta 3; Phospholipase C, gamma 1; Ephrin B1; Retinoblastoma-related gene; protein kinase C epsilon subspecies; Rat protein kinase C epsilon subspecies.; Spinocerebellar ataxia type 1; phospholipase A2, group V; Angiotensin I-converting enzyme (Dipeptidyl carboxypeptidase 1); Steroid sulfatase; protein kinase C zeta subspecies; Rat protein kinase C zeta subspecies.; Calcium channel alpha 1A; carcinoembryonic antigen-related cell adhesion molecule; amphiphysin; Rat glutathione S-transferase mRNA, complete cds; Cathepsin L; Acyl Coenzyme A dehydrogenase, long chain; ATP-binding cassette, sub-family B (MDR/TAP), member 1 (P-glycoprotein/multidrug resistance 1); c-fos protein (AA 1-380); Rat c-fos mRNA.; glutamate receptor, ionotropic, AMPA2 (alpha 2); syntaxin 6; dipeptidylpeptidase 6; G protein-coupled receptor kinase 2, groucho gene related (Drosophila); Max; protein kinase C alpha (AA 1-672); Rat mRNA for protein kinase C alpha.; fatty acid amide hydrolase; Carnitine palmitoyltransferase 1 alpha, liver isoform; calcium channel, voltage-dependent, L type, alpha 1D subunit; BRbeta B-regulatory subunit of protein phosphatase 2A; Secretogranin II; transmembrane receptor Unc5H2; potassium inwardly-rectifying channel, subfamily J, member 12; Acetylcholine receptor beta; B-cell translocation gene 1, anti-proliferative; Lectin, galactose binding, soluble 9 (Galectin-9); Insulin receptor; synaptotagmin 5; Rattus norvegicus calcium/calmodulin-dependent protein kinase II delta subunit mRNA, partial cds.; High mobility group 1; thyroid hormone receptor alpha; Rattus norvegicus cytochrome P450 4F5 (CYP4F5) mRNA, complete cds; Insulin-like growth factor 2 receptor; Rat glucagon receptor mRNA, complete cds; Arrestin, beta 1; protease (prosome, macropain) 26S subunit, ATPase 1; R. rattus mRNA for NPY-1 receptor.; kinase domain is 450..1295; Rattus rattus mRNA for PCTAIRE3, complete cds.; R. rattus RL/IF-1 mRNA.; Arrestin, beta 2; vascular endothelial growth factor; Ras-related small GTP binding protein 3A; Adenylyl cyclase 6; LIM motif-containing protein kinase 2; This sequence comes from FIG. 1b; A2 adenosine receptor [rats, striatum, mRNA, 2141 nt].; Adrenergic receptor kinase, beta 2 (G-protein-linked receptor kinase); Arrestin, beta 1; endothelial differentiation, sphingolipid G-protein-coupled receptor, 5; immediate early gene transcription factor NGFI-B; potassium inwardly-rectifying channel, subfamily J, member 12; Rattus norvegicus calcium/calmodulin-dependent protein kinase II delta subunit mRNA, partial cds.; interleukin 18; Max interacting protein 1; prostaglandin F2 receptor negative regulator; BRbeta B-regulatory subunit of protein phosphatase 2A; Protein tyrosine phosphatase, non-receptor type substrate 1 (SHP substrate 1); Rattus norvegicus insulin-regulated membrane aminopeptidase IRAP mRNA, complete cds; Ceruloplasmin (ferroxidase); cyclin-dependent kinase 5; adrenergic receptor kinase, beta 1; MAD (mothers against decapentaplegic, Drosophila) homolog 1; CamK I; calcium/calmodulin-dependent protein kinase type I +CaM-like protein kinase; Calcium channel alpha 1A; phosphofructokinase, muscle; p32-subunit of replication protein A; Rattus norvegicus mRNA for Janus protein tyrosine kinase 1, JAK1.; Rat insulin-like growth factor binding protein (rIGFBP-6) mRNA, complete cds.; Discoidin domain receptor (neurotrophic tyrosine kinase, receptor, type 4 (cell adhesion kinase)); Insulin-like growth factor 1 receptor; Tumor protein p53 (Li-Fraumeni syndrome); phospholipase A2, group VI; solute carrier family 2 (facilitated glucose transporter), member 5; Inhibitor of DNA binding 2, dominant negative helix-loop-helix protein; Rat mRNA for proteasome activator rPA28 subunit alpha, complete cds.; Protein tyrosine phosphatase, receptor type, A; aminopeptidase B; Rat mRNA for cyclin D1, complete cds.; syntaxin 5a; Natriuretic peptide receptor A/Guanylate cyclase A; TR4 orphan receptor; galanin receptor 2; casein kinase II, alpha 1 polypeptide; carcinoembryonic antigen-related cell adhesion molecule; protein tyrosine phosphatase, receptor type, R; Neurofibromatosis type 1; Rat glutathione S-transferase mRNA, complete cds; calcium channel, voltage-dependent, L type, alpha 1D subunit; Acetylcholine receptor alpha 3 (neuronal nicotine); mitogen activated protein kinase 3; mismatch repair protein; tissue inhibitor of metalloproteinase 2; Solute carrier family 4, member 2, anion exchange protein 2; Rat mRNA for multicatalytic proteionase (MCP) subunit L ingensin, Atp-dependent proteinase, proteasome, macropain).; Janus kinase 2 (a protein tyrosine kinase); kinase domain is 450..1295; Rattus rattus mRNA for PCTAIRE3, complete cds.; This sequence comes from FIG. 1; Na+/Cl(−)-dependent neurotransmitter transporter [rats, brain, mRNA, 3762 nt].; Set beta isoform; leukemogenesis protein; This sequence comes from FIG. 1 IIB; set=Set beta isoform {alternatively spliced} [rats, neonatal kidney, mRNA, 2026 nt].; synapsin II; Calmodulin III; subunit 8; R. rattus mRNA for glutathione transferase subunit 8.; Rattus norvegicus neuron-specific enolase (NSE) mRNA, complete cds; syntaxin 3; Tyrosine 3monooxygenase/tryptophan 5-monooxygenase activation protein, eta polypeptide; Carnitine palmitoyltransferase 1 alpha, liver isoform; Superoxide dismutase 1, soluble; phospholipase C, beta 3; Angiotensin I-converting enzyme (Dipeptidyl carboxypeptidase 1); c-fos protein (AA 1-380); Rat c-fos mRNA.; transmembrane receptor Unc5H2; GTPase Rab14; ATP-binding cassette, sub-family C (CFTR/MRP), member 1 (multiple drug resistance-associated protein); Inhibitor of DNA binding 1, helix-loop-helix protein (splice variation); and dipeptidylpeptidase 6, whereby the NM protein is expressed and neuronal cell death in the patient is stimulated.

33. The method of claim 32 wherein the patient has a neuronal tumor.

34. A method of screening for neuronal cell death in a patient, comprising:
detecting a neuronal marker (NM) protein selected from the group consisting of Ceruloplasmin (ferroxidase); Adenylyl cyclase 6; Insulin-like growth factor 1 receptor; vascular endothelial growth factor; Rat mRNA for sucrase isomaltase (EC 3.2.1.10).; Serotonin (5hydroxytryptamine (5HT)) receptor, type 1B; Fos like antigen 2; phospholipase C-III; Rat phospholipase C-III mRNA, complete cds.; interleukin 18; calcium channel, voltage-dependent, alpha2/delta subunit 1; Vesicle-associated membrane protein (synaptobrevin 2); putative; Rattus norvegicus G protein-coupled receptor (GPR19) gene, partial cds.; This sequence comes from FIG. 1b; A2 adenosine receptor [rats, striatum, mRNA, 2141 nt].; Max interacting protein 1; ATPase isoform 2, Na+K+ transporting, beta polypeptide 2; Secretory granule neuroendocrine, protein 1 (7B2 protein); Pim-1 oncogene; adenylate kinase 3; alpha-methylacyl-CoA racemase; Inhibitor of DNA binding 2, dominant negative helix-loop-helix protein; Rattus norvegicus mRNA for 20-alpha-hydroxysteroid dehydrogenase (20-alpha-HSD), complete cds; telomerase protein component 1; pyruvate dehydrogenase kinase, isoenzyme 1; Solute carrier family 4, member 2, anion exchange protein 2; phospholipase A2, group IIC; syntaxin 3; Rattus norvegicus mRNA for interleukin-4 receptor (soluble form), complete cds; Protein tyrosine phosphatase, non-receptor type substrate 1 (SHP substrate 1); B-cell translocation gene 2, anti-proliferative; Acyl-Coa dehydrogenase, Very long chain; Clusterin; syntaxin 4; Natriuretic peptide receptor A/Guanylate cyclase A; megakaryocyte-associated tyrosine kinase; presenilin-2; phospholipase A2, group VI; pancreatic lipase-related protein 2; phospholipase C, beta 3; Phospholipase C, gamma 1; Ephrin B1; Retinoblastoma-related gene; protein kinase C epsilon subspecies; Rat protein kinase C epsilon subspecies.; Spinocerebellar ataxia type 1; phospholipase A2, group V; Angiotensin I-converting enzyme (Dipeptidyl carboxypeptidase 1); Steroid sulfatase; protein kinase C zeta subspecies; Rat protein kinase C zeta subspecies.; Calcium channel alpha 1A; carcinoembryonic antigen-related cell adhesion molecule; amphiphysin; Rat glutathione S-transferase mRNA, complete cds; Cathepsin L; Acyl Coenzyme A dehydrogenase, long chain; ATP-binding cassette, sub-family B (MDR/TAP), member 1 (P-glycoprotein/multidrug resistance 1); c-fos protein (AA 1-380); Rat c-fos mRNA.; glutamate receptor, ionotropic, AMPA2 (alpha 2); syntaxin 6; dipeptidylpeptidase 6; G protein-coupled receptor kinase 2, groucho gene related (Drosophila); Max; protein kinase C alpha (AA 1-672); Rat mRNA for protein kinase C alpha.; fatty acid amide hydrolase; Carnitine palmitoyltransferase 1 alpha, liver isoform; calcium channel, voltage-dependent, L type, alpha 1D subunit; BRbeta B-regulatory subunit of protein phosphatase 2A; Secretogranin II; transmembrane receptor Unc5H2; potassium inwardly-rectifying channel, subfamily J, member 12; Acetylcholine receptor beta; B-cell translocation gene 1, anti-proliferative; Lectin, galactose binding, soluble 9 (Galectin-9); Insulin receptor; synaptotagmin 5; Rattus norvegicus calcium/calmodulin-dependent protein kinase II delta subunit mRNA, partial cds.; High mobility group 1; thyroid hormone receptor alpha; Rattus norvegicus cytochrome P450 4F5 (CYP4F5) mRNA, complete cds; Insulin-like growth factor 2 receptor; Rat glucagon receptor mRNA, complete cds; Arrestin, beta 1; protease (prosome, macropain) 26S subunit, ATPase 1; R. rattus mRNA for NPY-1 receptor.; kinase domain is 450..1295; Rattus rattus mRNA for PCTAIRE3, complete cds.; R. rattus RL/IF-1 mRNA.; Arrestin, beta 2; vascular endothelial growth factor; Ras-related small GTP binding protein 3A; Adenylyl cyclase 6; LIM motif-containing protein kinase 2; This sequence comes from FIG. 1b; A2 adenosine receptor [rats, striatum, mRNA, 2141 nt].; Adrenergic receptor kinase, beta 2 (G-protein-linked receptor kinase); Arrestin, beta 1; endothelial differentiation, sphingolipid G-protein-coupled receptor, 5; immediate early gene transcription factor NGFI-B; potassium inwardly-rectifying channel, subfamily J, member 12; Rattus norvegicus calcium/calmodulin-dependent protein kinase II delta subunit mRNA, partial cds.; interleukin 18; Max interacting protein 1; prostaglandin F2 receptor negative regulator; BRbeta B-regulatory subunit of protein phosphatase 2A; Protein tyrosine phosphatase, non-receptor type substrate 1 (SHP substrate 1); Rattus norvegicus insulin-regulated membrane aminopeptidase IRAP mRNA, complete cds; Ceruloplasmin (ferroxidase); cyclin-dependent kinase 5; adrenergic receptor kinase, beta 1; MAD (mothers against decapentaplegic, Drosophila) homolog 1; CamK I; calcium/calmodulin-dependent protein kinase type I +CaM-like protein kinase; Calcium channel alpha 1A; phosphofructokinase, muscle; p32-subunit of replication protein A; Rattus norvegicus mRNA for Janus protein tyrosine kinase 1, JAK1.; Rat insulin-like growth factor binding protein (rIGFBP-6) mRNA, complete cds.; Discoidin domain receptor (neurotrophic tyrosine kinase, receptor, type 4 (cell adhesion kinase)); Insulin-like growth factor 1 receptor; Tumor protein p53 (Li-Fraumeni syndrome); phospholipase A2, group VI; solute carrier family 2 (facilitated glucose transporter), member 5; Inhibitor of DNA binding 2, dominant negative helix-loop-helix protein; Rat mRNA for proteasome activator rPA28 subunit alpha, complete cds.; Protein tyrosine phosphatase, receptor type, A; aminopeptidase B; Rat mRNA for cyclin D1, complete cds.; syntaxin 5a; Natriuretic peptide receptor A/Guanylate cyclase A; TR4 orphan receptor; galanin receptor 2; casein kinase II, alpha 1 polypeptide; carcinoembryonic antigen-related cell adhesion molecule; protein tyrosine phosphatase, receptor type, R; Neurofibromatosis type 1; Rat glutathione S-transferase mRNA, complete cds; calcium channel, voltage-dependent, L type, alpha 1D subunit; Acetylcholine receptor alpha 3 (neuronal nicotine); mitogen activated protein kinase 3; mismatch repair protein; tissue inhibitor of metalloproteinase 2; Solute carrier family 4, member 2, anion exchange protein 2; Rat mRNA for multicatalytic proteionase (MCP) subunit L ingensin, Atp-dependent proteinase, proteasome, macropain).; Janus kinase 2 (a protein tyrosine kinase); kinase domain is 450..1295; Rattus rattus mRNA for PCTAIRE3, complete cds.; This sequence comes from FIG. 1; Na+/Cl(−)-dependent neurotransmitter transporter [rats, brain, mRNA, 3762 nt].; Set beta isoform; leukemogenesis protein; This sequence comes from FIG. 1 IIB; set=Set beta isoform {alternatively spliced} [rats, neonatal kidney, mRNA, 2026 nt].; synapsin II; Calmodulin III; subunit 8; R. rattus mRNA for glutathione transferase subunit 8.; Rattus norvegicus neuron-specific enolase (NSE) mRNA, complete cds; syntaxin 3; Tyrosine 3monooxygenase/tryptophan 5-monooxygenase activation protein, eta polypeptide; Carnitine palmitoyltransferase 1 alpha, liver isoform; Superoxide dismutase 1, soluble; phospholipase C, beta 3; Angiotensin I-converting enzyme (Dipeptidyl carboxypeptidase 1); c-fos protein (AA 1-380); Rat c-fos mRNA.; transmembrane receptor Unc5H2; GTPase Rab14; ATP-binding cassette, sub-family C (CFTR/MRP), member 1 (multiple drug resistance-associated protein); Inhibitor of DNA binding 1, helix-loop-helix protein (splice variation); and dipeptidylpeptidase 6, in a body fluid collected from the patient, wherein detection of the NM protein indicates neuronal cell death in the patient.

35. A method of screening for neuronal cell death in a patient, comprising:
detecting in a body fluid collected from the patient a nucleic acid encoding a neuronal marker (NM) protein selected from the group consisting of: Ceruloplasmin (ferroxidase); Adenylyl cyclase 6; Insulin-like growth factor 1 receptor; vascular endothelial growth factor; Rat mRNA for sucrase isomaltase (EC 3.2.1.10).; Serotonin (5-hydroxytryptamine (5HT)) receptor, type 1B; Fos like antigen 2; phospholipase C-III; Rat phospholipase C-III mRNA, complete cds.; interleukin 18; calcium channel, voltage-dependent, alpha2/delta subunit 1; Vesicle-associated membrane protein (synaptobrevin 2); putative; Rattus norvegicus G protein-coupled receptor (GPR19) gene, partial cds.; This sequence comes from FIG. 1b; A2 adenosine receptor [rats, striatum, mRNA, 2141 nt].; Max interacting protein 1; ATPase isoform 2, Na+K+ transporting, beta polypeptide 2; Secretory granule neuroendocrine, protein 1 (7B2 protein); Pim-1 oncogene; adenylate kinase 3; alpha-methylacyl-CoA racemase; Inhibitor of DNA binding 2, dominant negative helix-loop-helix protein; Rattus norvegicus mRNA for 20-alpha-hydroxysteroid dehydrogenase (20-alpha-HSD), complete cds; telomerase protein component 1; pyruvate dehydrogenase kinase, isoenzyme 1; Solute carrier family 4, member 2, anion exchange protein 2; phospholipase A2, group IIC; syntaxin 3; Rattus norvegicus mRNA for interleukin-4 receptor (soluble form), complete cds; Protein tyrosine phosphatase, non-receptor type substrate 1 (SHP substrate 1); B-cell translocation gene 2, anti-proliferative; Acyl-Coa dehydrogenase, Very long chain; Clusterin; syntaxin 4; Natriuretic peptide receptor A/Guanylate cyclase A; megakaryocyte-associated tyrosine kinase; presenilin-2; phospholipase A2, group VI; pancreatic lipase-related protein 2; phospholipase C, beta 3; Phospholipase C, gamma 1; Ephrin B1; Retinoblastoma-related gene; protein kinase C epsilon subspecies; Rat protein kinase C epsilon subspecies.; Spinocerebellar ataxia type 1; phospholipase A2, group V; Angiotensin I-converting enzyme (Dipeptidyl carboxypeptidase 1); Steroid sulfatase; protein kinase C zeta subspecies; Rat protein kinase C zeta subspecies.; Calcium channel alpha 1A; carcinoembryonic antigen-related cell adhesion molecule; amphiphysin; Rat glutathione S-transferase mRNA, complete cds; Cathepsin L; Acyl Coenzyme A dehydrogenase, long chain; ATP-binding cassette, sub-family B (MDR/TAP), member 1 (P-glycoprotein/multidrug resistance 1); c-fos protein (AA 1-380); Rat c-fos mRNA.; glutamate receptor, ionotropic, AMPA2 (alpha 2); syntaxin 6; dipeptidylpeptidase 6; G protein-coupled receptor kinase 2, groucho gene related (Drosophila); Max; protein kinase C alpha (AA 1-672); Rat mRNA for protein kinase C alpha.; fatty acid amide hydrolase; Carnitine palmitoyltransferase 1 alpha, liver isoform; calcium channel, voltage-dependent, L type, alpha 1D subunit; BRbeta B-regulatory subunit of protein phosphatase 2A; Secretogranin II; transmembrane receptor Unc5H2; potassium inwardly-rectifying channel, subfamily J, member 12; Acetylcholine receptor beta; B-cell translocation gene 1, anti-proliferative; Lectin, galactose binding, soluble 9 (Galectin-9); Insulin receptor; synaptotagmin 5; Rattus norvegicus calcium/calmodulin-dependent protein kinase II delta subunit mRNA, partial cds.; High mobility group 1; thyroid hormone receptor alpha; Rattus norvegicus cytochrome P450 4F5 (CYP4F5) mRNA, complete cds; Insulin-like growth factor 2 receptor; Rat glucagon receptor mRNA, complete cds; Arrestin, beta 1; protease (prosome, macropain) 26S subunit, ATPase 1; R. rattus mRNA for NPY-1 receptor.; kinase domain is 450..1295; Rattus rattus mRNA for PCTAIRE3, complete cds.; R. rattus RL/IF-1 mRNA.; Arrestin, beta 2; vascular endothelial growth factor; Ras-related small GTP binding protein 3A; Adenylyl cyclase 6; LIM motif-containing protein kinase 2; This sequence comes from FIG. 1b; A2 adenosine receptor [rats, striatum, mRNA, 2141 nt].; Adrenergic receptor kinase, beta 2 (G-protein-linked receptor kinase); Arrestin, beta 1; endothelial differentiation, sphingolipid G-protein-coupled receptor, 5; immediate early gene transcription factor NGFI-B; potassium inwardly-rectifying channel, subfamily J, member 12; Rattus norvegicus calcium/calmodulin-dependent protein kinase II delta subunit mRNA, partial cds.; interleukin 18; Max interacting protein 1; prostaglandin F2 receptor negative regulator; BRbeta B-regulatory subunit of protein phosphatase 2A; Protein tyrosine phosphatase, non-receptor type substrate 1 (SHP substrate 1); Rattus norvegicus insulin-regulated membrane aminopeptidase IRAP mRNA, complete cds; Ceruloplasmin (ferroxidase); cyclin-dependent kinase 5; adrenergic receptor kinase, beta 1; MAD (mothers against decapentaplegic, Drosophila) homolog 1; CamK I; calcium/calmodulin-dependent protein kinase type I +CaM-like protein kinase; Calcium channel alpha 1A; phosphofructokinase, muscle; p32-subunit of replication protein A; Rattus norvegicus mRNA for Janus protein tyrosine kinase 1, JAK1.; Rat insulin-like growth factor binding protein (rIGFBP-6) mRNA, complete cds.; Discoidin domain receptor (neurotrophic tyrosine kinase, receptor, type 4 (cell adhesion kinase)); Insulin-like growth factor 1 receptor; Tumor protein p53 (Li-Fraumeni syndrome); phospholipase A2, group VI; solute carrier family 2 (facilitated glucose transporter), member 5; Inhibitor of DNA binding 2, dominant negative helix-loop-helix protein; Rat mRNA for proteasome activator rPA28 subunit alpha, complete cds.; Protein tyrosine phosphatase, receptor type, A; aminopeptidase B; Rat mRNA for cyclin D1, complete cds.; syntaxin 5a; Natriuretic peptide receptor A/Guanylate cyclase A; TR4 orphan receptor; galanin receptor 2; casein kinase II, alpha 1 polypeptide; carcinoembryonic antigen-related cell adhesion molecule; protein tyrosine phosphatase, receptor type, R; Neurofibromatosis type 1; Rat glutathione S-transferase mRNA, complete cds; calcium channel, voltage-dependent, L type, alpha 1D subunit; Acetylcholine receptor alpha 3 (neuronal nicotine); mitogen activated protein kinase 3; mismatch repair protein; tissue inhibitor of metalloproteinase 2; Solute carrier family 4, member 2, anion exchange protein 2; Rat mRNA for multicatalytic proteionase (MCP) subunit L ingensin, Atp-dependent proteinase, proteasome, macropain).; Janus kinase 2 (a protein tyrosine kinase); kinase domain is 450..1295; Rattus rattus mRNA for PCTAIRE3, complete cds.; This sequence comes from FIG. 1; Na+/Cl(−)-dependent neurotransmitter transporter [rats, brain, mRNA, 3762 nt].; Set beta isoform; leukemogenesis protein; This sequence comes from FIG. 1 IIB; set=Set beta isoform {alternatively spliced} [rats, neonatal kidney, mRNA, 2026 nt].; synapsin II; Calmodulin III; subunit 8; R. rattus mRNA for glutathione transferase subunit 8.; Rattus norvegicus neuron-specific enolase (NSE) mRNA, complete cds; syntaxin 3; Tyrosine 3monooxygenase/tryptophan 5-monooxygenase activation protein, eta polypeptide; Carnitine palmitoyltransferase 1 alpha, liver isoform; Superoxide dismutase 1, soluble; phospholipase C, beta 3; Angiotensin I-converting enzyme (Dipeptidyl carboxypeptidase 1); c-fos protein (AA 1-380); Rat c-fos mRNA.; transmembrane receptor Unc5H2; GTPase Rab14; ATP-binding cassette, sub-family C (CFTR/MRP), member 1 (multiple drug resistance-associated protein); Inhibitor of DNA binding 1, helix-loop-helix protein (splice variation); and dipeptidylpeptidase 6, wherein detection of the NM protein indicates neuronal cell death in the patient.

36. A method to identify candidate drugs for treating neuronal cell death, comprising:
contacting cells which express one or more neuronal marker (NM) genes selected from the group consisting of: Ceruloplasmin (ferroxidase); Adenylyl cyclase 6; Insulin-like growth factor 1 receptor; vascular endothelial growth factor; Rat mRNA for sucrase isomaltase (EC 3.2.1.10).; Serotonin (5-hydroxytryptamine (5HT)) receptor, type 1B; Fos like antigen 2; phospholipase C-III; Rat phospholipase C-III mRNA, complete cds.; interleukin 18; calcium channel, voltage-dependent, alpha2/delta subunit 1; Vesicle-associated membrane protein (synaptobrevin 2); putative; Rattus norvegicus G protein-coupled receptor (GPR19) gene, partial cds.; This sequence comes from FIG. 1b; A2 adenosine receptor [rats, striatum, mRNA, 2141 nt].; Max interacting protein 1; ATPase isoform 2, Na+K+ transporting, beta polypeptide 2; Secretory granule neuroendocrine, protein 1 (7B2 protein); Pim-1 oncogene; adenylate kinase 3; alpha-methylacyl-CoA racemase; Inhibitor of DNA binding 2, dominant negative helix-loop-helix protein; Rattus norvegicus mRNA for 20-alpha-hydroxysteroid dehydrogenase (20-alpha-HSD), complete cds; telomerase protein component 1; pyruvate dehydrogenase kinase, isoenzyme 1; Solute carrier family 4, member 2, anion exchange protein 2; phospholipase A2, group IIC; syntaxin 3; Rattus norvegicus mRNA for interleukin-4 receptor (soluble form), complete cds; Protein tyrosine phosphatase, non-receptor type substrate 1 (SHP substrate 1); B-cell translocation gene 2, anti-proliferative; Acyl-Coa dehydrogenase, Very long chain; Clusterin; syntaxin 4; Natriuretic peptide receptor A/Guanylate cyclase A; megakaryocyte-associated tyrosine kinase; presenilin-2; phospholipase A2, group VI; pancreatic lipase-related protein 2; phospholipase C, beta 3; Phospholipase C, gamma 1; Ephrin B1; Retinoblastoma-related gene; protein kinase C epsilon subspecies; Rat protein kinase C epsilon subspecies.; Spinocerebellar ataxia type 1; phospholipase A2, group V; Angiotensin I-converting enzyme (Dipeptidyl carboxypeptidase 1); Steroid sulfatase; protein kinase C zeta subspecies; Rat protein kinase C zeta subspecies.; Calcium channel alpha 1A; carcinoembryonic antigen-related cell adhesion molecule; amphiphysin; Rat glutathione S-transferase mRNA, complete cds; Cathepsin L; Acyl Coenzyme A dehydrogenase, long chain; ATP-binding cassette, sub-family B (MDR/TAP), member 1 (P-glycoprotein/multidrug resistance 1); c-fos protein (AA 1-380); Rat c-fos mRNA.; glutamate receptor, ionotropic, AMPA2 (alpha 2); syntaxin 6; dipeptidylpeptidase 6; G protein-coupled receptor kinase 2, groucho gene related (Drosophila); Max; protein kinase C alpha (AA 1-672); Rat mRNA for protein kinase C alpha.; fatty acid amide hydrolase; Carnitine palmitoyltransferase 1 alpha, liver isoform; calcium channel, voltage-dependent, L type, alpha 1D subunit; BRbeta B-regulatory subunit of protein phosphatase 2A; Secretogranin II; transmembrane receptor Unc5H2; potassium inwardly-rectifying channel, subfamily J, member 12; Acetylcholine receptor beta; B-cell translocation gene 1, anti-proliferative; Lectin, galactose binding, soluble 9 (Galectin-9); Insulin receptor; synaptotagmin 5; Rattus norvegicus calcium/calmodulin-dependent protein kinase II delta subunit mRNA, partial cds.; High mobility group 1; thyroid hormone receptor alpha; Rattus norvegicus cytochrome P450 4F5 (CYP4F5) mRNA, complete cds; Insulin-like growth factor 2 receptor; Rat glucagon receptor mRNA, complete cds; Arrestin, beta 1; protease (prosome, macropain) 26S subunit, ATPase 1; R. rattus mRNA for NPY-1 receptor.; kinase domain is 450..1295; Rattus rattus mRNA for PCTAIRE3, complete cds.; R. rattus RL/IF-1 mRNA.; Arrestin, beta 2; vascular endothelial growth factor; Ras-related small GTP binding protein 3A; Adenylyl cyclase 6; LIM motif-containing protein kinase 2; This sequence comes from FIG. 1b; A2 adenosine receptor [rats, striatum, mRNA, 2141 nt].; Adrenergic receptor kinase, beta 2 (G-protein-linked receptor kinase); Arrestin, beta 1; endothelial differentiation, sphingolipid G-protein-coupled receptor, 5; immediate early gene transcription factor NGFI-B; potassium inwardly-rectifying channel, subfamily J, member 12; Rattus norvegicus calcium/calmodulin-dependent protein kinase II delta subunit mRNA, partial cds.; interleukin 18; Max interacting protein 1; prostaglandin F2 receptor negative regulator; BRbeta B-regulatory subunit of protein phosphatase 2A; Protein tyrosine phosphatase, non-receptor type substrate 1 (SHP substrate 1); Rattus norvegicus insulin-regulated membrane aminopeptidase IRAP mRNA, complete cds; Ceruloplasmin (ferroxidase); cyclin-dependent kinase 5; adrenergic receptor kinase, beta 1; MAD (mothers against decapentaplegic, Drosophila) homolog 1; CamK I; calcium/calmodulin-dependent protein kinase type I +CaM-like protein kinase; Calcium channel alpha 1A; phosphofructokinase, muscle; p32-subunit of replication protein A; Rattus norvegicus mRNA for Janus protein tyrosine kinase 1, JAK1.; Rat insulin-like growth factor binding protein (rIGFBP-6) mRNA, complete cds.; Discoidin domain receptor (neurotrophic tyrosine kinase, receptor, type 4 (cell adhesion kinase)); Insulin-like growth factor 1 receptor; Tumor protein p53 (Li-Fraumeni syndrome); phospholipase A2, group VI; solute carrier family 2 (facilitated glucose transporter), member 5; Inhibitor of DNA binding 2, dominant negative helix-loop-helix protein; Rat mRNA for proteasome activator rPA28 subunit alpha, complete cds.; Protein tyrosine phosphatase, receptor type, A; aminopeptidase B; Rat mRNA for cyclin D1, complete cds.; syntaxin 5a; Natriuretic peptide receptor A/Guanylate cyclase A; TR4 orphan receptor; galanin receptor 2; casein kinase II, alpha 1 polypeptide; carcinoembryonic antigen-related cell adhesion molecule; protein tyrosine phosphatase, receptor type, R; Neurofibromatosis type 1; Rat glutathione S-transferase mRNA, complete cds; calcium channel, voltage-dependent, L type, alpha 1D subunit; Acetylcholine receptor alpha 3 (neuronal nicotine); mitogen activated protein kinase 3; mismatch repair protein; tissue inhibitor of metalloproteinase 2; Solute carrier family 4, member 2, anion exchange protein 2; Rat mRNA for multicatalytic proteionase (MCP) subunit L ingensin, Atp-dependent proteinase, proteasome, macropain).; Janus kinase 2 (a protein tyrosine kinase); kinase domain is 450..1295; Rattus rattus mRNA for PCTAIRE3, complete cds.; This sequence comes from FIG. 1; Na+/Cl(−)-dependent neurotransmitter transporter [rats, brain, mRNA, 3762 nt].; Set beta isoform; leukemogenesis protein; This sequence comes from FIG. 1 IIB; set=Set beta isoform {alternatively spliced} [rats, neonatal kidney, mRNA, 2026 nt].; synapsin II; Calmodulin III; subunit 8; R. rattus mRNA for glutathione transferase subunit 8.; Rattus norvegicus neuron-specific enolase (NSE) mRNA, complete cds; syntaxin 3; Tyrosine 3monooxygenase/tryptophan 5-monooxygenase activation protein, eta polypeptide; Carnitine palmitoyltransferase 1 alpha, liver isoform; Superoxide dismutase 1, soluble; phospholipase C, beta 3; Angiotensin I-converting enzyme (Dipeptidyl carboxypeptidase 1); c-fos protein (AA 1-380); Rat c-fos mRNA.; transmembrane receptor Unc5H2; GTPase Rab14; ATP-binding cassette, sub-family C (CFTR/MRP), member 1 (multiple drug resistance-associated protein); Inhibitor of DNA binding 1, helix-loop-helix protein (splice variation); and dipeptidylpeptidase 6, with a test compound;
determining expression of said one or more NM genes by hybridization of mRNA of said cells to a nucleic acid probe which is complementary to said mRNA; and
identifying a test compound as a candidate drug for treating neuronal cell death if it decreases expression of said one or more NM genes.

37. The method of claim 36 wherein the cells are retinal cells.

38. The method of claim 36 wherein the cells are recombinant host cells which are transfected with an expression construct which encodes said one or more NMs.

39. A method to identify candidate drugs for treating neuronal cell death, comprising:
contacting cells which express one or more neuronal marker (NM) proteins selected from the group consisting of: Ceruloplasmin (ferroxidase); Adenylyl cyclase 6; Insulin-like growth factor 1 receptor; vascular endothelial growth factor; Rat mRNA for sucrase isomaltase (EC 3.2.1.10).; Serotonin (5-hydroxytryptamine (5HT)) receptor, type 1B; Fos like antigen 2; phospholipase C-III; Rat phospholipase C-III mRNA, complete cds.; interleukin 18; calcium channel, voltage-dependent, alpha2/delta subunit 1; Vesicle-associated membrane protein (synaptobrevin 2); putative; Rattus norvegicus G protein-coupled receptor (GPR19) gene, partial cds.; This sequence comes from FIG. 1b; A2 adenosine receptor [rats, striatum, mRNA, 2141 nt].; Max interacting protein 1; ATPase isoform 2, Na+K+ transporting, beta polypeptide 2; Secretory granule neuroendocrine, protein 1 (7B2 protein); Pim-1 oncogene; adenylate kinase 3; alpha-methylacyl-CoA racemase; Inhibitor of DNA binding 2, dominant negative helix-loop-helix protein; Rattus norvegicus mRNA for 20-alpha-hydroxysteroid dehydrogenase (20-alpha-HSD), complete cds; telomerase protein component 1; pyruvate dehydrogenase kinase, isoenzyme 1; Solute carrier family 4, member 2, anion exchange protein 2; phospholipase A2, group IIC; syntaxin 3; Rattus norvegicus mRNA for interleukin-4 receptor (soluble form), complete cds; Protein tyrosine phosphatase, non-receptor type substrate 1 (SHP substrate 1); B-cell translocation gene 2, anti-proliferative; Acyl-Coa dehydrogenase, Very long chain; Clusterin; syntaxin 4; Natriuretic peptide receptor A/Guanylate cyclase A; megakaryocyte-associated tyrosine kinase; presenilin-2; phospholipase A2, group VI; pancreatic lipase-related protein 2; phospholipase C, beta 3; Phospholipase C, gamma 1; Ephrin B1; Retinoblastoma-related gene; protein kinase C epsilon subspecies; Rat protein kinase C epsilon subspecies.; Spinocerebellar ataxia type 1; phospholipase A2, group V; Angiotensin I-converting enzyme (Dipeptidyl carboxypeptidase 1); Steroid sulfatase; protein kinase C zeta subspecies; Rat protein kinase C zeta subspecies.; Calcium channel alpha 1A; carcinoembryonic antigen-related cell adhesion molecule; amphiphysin; Rat glutathione S-transferase mRNA, complete cds; Cathepsin L; Acyl Coenzyme A dehydrogenase, long chain; ATP-binding cassette, sub-family B (MDR/TAP), member 1 (P-glycoprotein/multidrug resistance 1); c-fos protein (AA 1-380); Rat c-fos mRNA.; glutamate receptor, ionotropic, AMPA2 (alpha 2); syntaxin 6; dipeptidylpeptidase 6; G protein-coupled receptor kinase 2, groucho gene related (Drosophila); Max; protein kinase C alpha (AA 1-672); Rat mRNA for protein kinase C alpha.; fatty acid amide hydrolase; Carnitine palmitoyltransferase 1 alpha, liver isoform; calcium channel, voltage-dependent, L type, alpha 1D subunit; BRbeta B-regulatory subunit of protein phosphatase 2A; Secretogranin II; transmembrane receptor Unc5H2; potassium inwardly-rectifying channel, subfamily J, member 12; Acetylcholine receptor beta; B-cell translocation gene 1, anti-proliferative; Lectin, galactose binding, soluble 9 (Galectin-9); Insulin receptor; synaptotagmin 5; Rattus norvegicus calcium/calmodulin-dependent protein kinase II delta subunit mRNA, partial cds.; High mobility group 1; thyroid hormone receptor alpha; Rattus norvegicus cytochrome P450 4F5 (CYP4F5) mRNA, complete cds; Insulin-like growth factor 2 receptor; Rat glucagon receptor mRNA, complete cds; Arrestin, beta 1; protease (prosome, macropain) 26S subunit, ATPase 1; R. rattus mRNA for NPY-1 receptor.; kinase domain is 450..1295; Rattus rattus mRNA for PCTAIRE3, complete cds.; R. rattus RL/IF-1 mRNA.; Arrestin, beta 2; vascular endothelial growth factor; Ras-related small GTP binding protein 3A; Adenylyl cyclase 6; LIM motif-containing protein kinase 2; This sequence comes from FIG. 1b; A2 adenosine receptor [rats, striatum, mRNA, 2141 nt].; Adrenergic receptor kinase, beta 2 (G-protein-linked receptor kinase); Arrestin, beta 1; endothelial differentiation, sphingolipid G-protein-coupled receptor, 5; immediate early gene transcription factor NGFI-B; potassium inwardly-rectifying channel, subfamily J, member 12; Rattus norvegicus calcium/calmodulin-dependent protein kinase II delta subunit mRNA, partial cds.; interleukin 18; Max interacting protein 1; prostaglandin F2 receptor negative regulator; BRbeta B-regulatory subunit of protein phosphatase 2A; Protein tyrosine phosphatase, non-receptor type substrate 1 (SHP substrate 1); Rattus norvegicus insulin-regulated membrane aminopeptidase IRAP mRNA, complete cds; Ceruloplasmin (ferroxidase); cyclin-dependent kinase 5; adrenergic receptor kinase, beta 1; MAD (mothers against decapentaplegic, Drosophila) homolog 1; CamK I; calcium/calmodulin-dependent protein kinase type I +CaM-like protein kinase; Calcium channel alpha 1A; phosphofructokinase, muscle; p32-subunit of replication protein A; Rattus norvegicus mRNA for Janus protein tyrosine kinase 1, JAK1.; Rat insulin-like growth factor binding protein (rIGFBP-6) mRNA, complete cds.; Discoidin domain receptor (neurotrophic tyrosine kinase, receptor, type 4 (cell adhesion kinase)); Insulin-like growth factor 1 receptor; Tumor protein p53 (Li-Fraumeni syndrome); phospholipase A2, group VI; solute carrier family 2 (facilitated glucose transporter), member 5; Inhibitor of DNA binding 2, dominant negative helix-loop-helix protein; Rat mRNA for proteasome activator rPA28 subunit alpha, complete cds.; Protein tyrosine phosphatase, receptor type, A; aminopeptidase B; Rat mRNA for cyclin D1, complete cds.; syntaxin 5a; Natriuretic peptide receptor A/Guanylate cyclase A; TR4 orphan receptor; galanin receptor 2; casein kinase II, alpha 1 polypeptide; carcinoembryonic antigen-related cell adhesion molecule; protein tyrosine phosphatase, receptor type, R; Neurofibromatosis type 1; Rat glutathione S-transferase mRNA, complete cds; calcium channel, voltage-dependent, L type, alpha 1D subunit; Acetylcholine receptor alpha 3 (neuronal nicotine); mitogen activated protein kinase 3; mismatch repair protein; tissue inhibitor of metalloproteinase 2; Solute carrier family 4, member 2, anion exchange protein 2; Rat mRNA for multicatalytic proteionase (MCP) subunit L ingensin, Atp-dependent proteinase, proteasome, macropain).; Janus kinase 2 (a protein tyrosine kinase); kinase domain is 450..1295; Rattus rattus mRNA for PCTAIRE3, complete cds.; This sequence comes from FIG. 1; Na+/Cl(−)-dependent neurotransmitter transporter [rats, brain, mRNA, 3762 nt].; Set beta isoform; leukemogenesis protein; This sequence comes from FIG. 11B; set=Set beta isoform {alternatively spliced} [rats, neonatal kidney, mRNA, 2026 nt].; synapsin II; Calmodulin III; subunit 8; R. rattus mRNA for glutathione transferase subunit 8.; Rattus norvegicus neuron-specific enolase (NSE) mRNA, complete cds; syntaxin 3; Tyrosine 3monooxygenase/tryptophan 5-monooxygenase activation protein, eta polypeptide; Carnitine palmitoyltransferase 1 alpha, liver isoform; Superoxide dismutase 1, soluble; phospholipase C, beta 3; Angiotensin I-converting enzyme (Dipeptidyl carboxypeptidase 1); c-fos protein (AA 1-380); Rat c-fos mRNA.; transmembrane receptor Unc5H2; GTPase Rab14; ATP-binding cassette, sub-family C (CFTR/MRP), member 1 (multiple drug resistance-associated protein); Inhibitor of DNA binding 1, helix-loop-helix protein (splice variation); and dipeptidylpeptidase 6, with a test compound;
determining amount of said one or more NM proteins in said cells; and
identifying a test compound as a candidate drug for treating tumors if it decreases the amount of one more NM proteins in said cells.

40. The method of claim 39 wherein the cells are retinal cells.

41. The method of claim 39 wherein the cells are recombinant host cells which are transfected with an expression construct which encodes said one or more NMs.

42. A method to identify candidate drugs for treating neuronal cell death, comprising:
contacting cells which express one or more neuronal marker (NM) proteins selected from the group consisting of: Ceruloplasmin (ferroxidase); Adenylyl cyclase 6; Insulin-like growth factor 1 receptor; vascular endothelial growth factor; Rat mRNA for sucrase isomaltase (EC 3.2.11.0).; Serotonin (5-hydroxytryptamine (5HT)) receptor, type 1B; Fos like antigen 2; phospholipase C-III; Rat phospholipase C-III mRNA, complete cds.; interleukin 18; calcium channel, voltage-dependent, alpha2/delta subunit 1; Vesicle-associated membrane protein (synaptobrevin 2); putative; Rattus norvegicus G protein-coupled receptor (GPR19) gene, partial cds.; This sequence comes from FIG. 1b; A2 adenosine receptor [rats, striatum, mRNA, 2141 nt].; Max interacting protein 1; ATPase isoform 2, Na+K+ transporting, beta polypeptide 2; Secretory granule neuroendocrine, protein 1 (7B2 protein); Pim-1 oncogene; adenylate kinase 3; alpha-methylacyl-CoA racemase; Inhibitor of DNA binding 2, dominant negative helix-loop-helix protein; Rattus norvegicus mRNA for 20-alpha-hydroxysteroid dehydrogenase (20-alpha-HSD), complete cds; telomerase protein component 1; pyruvate dehydrogenase kinase, isoenzyme 1; Solute carrier family 4, member 2, anion exchange protein 2; phospholipase A2, group IIC; syntaxin 3; Rattus norvegicus mRNA for interleukin-4 receptor (soluble form), complete cds; Protein tyrosine phosphatase, non-receptor type substrate 1 (SHP substrate 1); B-cell translocation gene 2, anti-proliferative; Acyl-Coa dehydrogenase, Very long chain; Clusterin; syntaxin 4; Natriuretic peptide receptor A/Guanylate cyclase A; megakaryocyte-associated tyrosine kinase; presenilin-2; phospholipase A2, group VI; pancreatic lipase-related protein 2; phospholipase C, beta 3; Phospholipase C, gamma 1; Ephrin B1; Retinoblastoma-related gene; protein kinase C epsilon subspecies; Rat protein kinase C epsilon subspecies.; Spinocerebellar ataxia type 1; phospholipase A2, group V; Angiotensin I-converting enzyme (Dipeptidyl carboxypeptidase 1); Steroid sulfatase; protein kinase C zeta subspecies; Rat protein kinase C zeta subspecies.; Calcium channel alpha 1A; carcinoembryonic antigen-related cell adhesion molecule; amphiphysin; Rat glutathione S-transferase mRNA, complete cds; Cathepsin L; Acyl Coenzyme A dehydrogenase, long chain; ATP-binding cassette, sub-family B (MDR/TAP), member 1 (P-glycoprotein/multidrug resistance 1); c-fos protein (AA 1-380); Rat c-fos mRNA.; glutamate receptor, ionotropic, AMPA2 (alpha 2); syntaxin 6; dipeptidylpeptidase 6; G protein-coupled receptor kinase 2, groucho gene related (Drosophila); Max; protein kinase C alpha (AA 1-672); Rat mRNA for protein kinase C alpha.; fatty acid amide hydrolase; Carnitine palmitoyltransferase 1 alpha, liver isoform; calcium channel, voltage-dependent, L type, alpha 1D subunit; BRbeta B-regulatory subunit of protein phosphatase 2A; Secretogranin II; transmembrane receptor Unc5H2; potassium inwardly-rectifying channel, subfamily J, member 12; Acetylcholine receptor beta; B-cell translocation gene 1, anti-proliferative; Lectin, galactose binding, soluble 9 (Galectin-9); Insulin receptor; synaptotagmin 5; Rattus norvegicus calcium/calmodulin-dependent protein kinase II delta subunit mRNA, partial cds.; High mobility group 1; thyroid hormone receptor alpha; Rattus norvegicus cytochrome P450 4F5 (CYP4F5) mRNA, complete cds; Insulin-like growth factor 2 receptor; Rat glucagon receptor mRNA, complete cds; Arrestin, beta 1; protease (prosome, macropain) 26S subunit, ATPase 1; R. rattus mRNA for NPY-1 receptor.; kinase domain is 450..1295; Rattus rattus mRNA for PCTAIRE3, complete cds.; R. rattus RL/IF-1 mRNA.; Arrestin, beta 2; vascular endothelial growth factor; Ras-related small GTP binding protein 3A; Adenylyl cyclase 6; LIM motif-containing protein kinase 2; This sequence comes from FIG. 1b; A2 adenosine receptor [rats, striatum, mRNA, 2141 nt].; Adrenergic receptor kinase, beta 2 (G-protein-linked receptor kinase); Arrestin, beta 1; endothelial differentiation, sphingolipid G-protein-coupled receptor, 5; immediate early gene transcription factor NGFI-B; potassium inwardly-rectifying channel, subfamily J, member 12; Rattus norvegicus calcium/calmodulin-dependent protein kinase II delta subunit mRNA, partial cds.; interleukin 18; Max interacting protein 1; prostaglandin F2 receptor negative regulator; BRbeta B-regulatory subunit of protein phosphatase 2A; Protein tyrosine phosphatase, non-receptor type substrate 1 (SHP substrate 1); Rattus norvegicus insulin-regulated membrane aminopeptidase IRAP mRNA, complete cds; Ceruloplasmin (ferroxidase); cyclin-dependent kinase 5; adrenergic receptor kinase, beta 1; MAD (mothers against decapentaplegic, Drosophila) homolog 1; CamK I; calcium/calmodulin-dependent protein kinase type I +CaM-like protein kinase; Calcium channel alpha 1A; phosphofructokinase, muscle; p32-subunit of replication protein A; Rattus norvegicus mRNA for Janus protein tyrosine kinase 1, JAK1.; Rat insulin-like growth factor binding protein (rIGFBP-6) mRNA, complete cds.; Discoidin domain receptor (neurotrophic tyrosine kinase, receptor, type 4 (cell adhesion kinase)); Insulin-like growth factor 1 receptor; Tumor protein p53 (Li-Fraumeni syndrome); phospholipase A2, group VI; solute carrier family 2 (facilitated glucose transporter), member 5; Inhibitor of DNA binding 2, dominant negative helix-loop-helix protein; Rat mRNA for proteasome activator rPA28 subunit alpha, complete cds.; Protein tyrosine phosphatase, receptor type, A; aminopeptidase B; Rat mRNA for cyclin D1, complete cds.; syntaxin Sa; Natriuretic peptide receptor A/Guanylate cyclase A; TR4 orphan receptor; galanin receptor 2; casein kinase II, alpha 1 polypeptide; carcinoembryonic antigen-related cell adhesion molecule; protein tyrosine phosphatase, receptor type, R; Neurofibromatosis type 1; Rat glutathione S-transferase mRNA, complete cds; calcium channel, voltage-dependent, L type, alpha 1D subunit; Acetylcholine receptor alpha 3 (neuronal nicotine); mitogen activated protein kinase 3; mismatch repair protein; tissue inhibitor of metalloproteinase 2; Solute carrier family 4, member 2, anion exchange protein 2; Rat mRNA for multicatalytic proteionase (MCP) subunit L ingensin, Atp-dependent proteinase, proteasome, macropain).; Janus kinase 2 (a protein tyrosine kinase); kinase domain is 450..1295; Rattus rattus mRNA for PCTAIRE3, complete cds.; This sequence comes from FIG. 1; Na+/Cl(−)-dependent neurotransmitter transporter [rats, brain, mRNA, 3762 nt].; Set beta isoform; leukemogenesis protein; This sequence comes from FIG. 1 IIB; set=Set beta isoform {alternatively spliced} [rats, neonatal kidney, mRNA, 2026 nt].; synapsin II; Calmodulin III; subunit 8; R. rattus mRNA for glutathione transferase subunit 8.; Rattus norvegicus neuron-specific enolase (NSE) mRNA, complete cds; syntaxin 3; Tyrosine 3-monooxygenase/tryptophan 5-monooxygenase activation protein, eta polypeptide; Carnitine palmitoyltransferase 1 alpha, liver isoform; Superoxide dismutase 1, soluble; phospholipase C, beta 3; Angiotensin I-converting enzyme (Dipeptidyl carboxypeptidase 1); c-fos protein (AA 1-380); Rat c-fos mRNA.; transmembrane receptor Unc5H2; GTPase Rab14; ATP-binding cassette, sub-family C (CFTR/MRP), member 1 (multiple drug resistance-associated protein); Inhibitor of DNA binding 1, helix-loop-helix protein (splice variation); and dipeptidylpeptidase 6 with a test compound;
determining activity of said one or more NM proteins in said cells; and
identifying a test compound as a candidate drug for treating neuronal cell death if it decreases the activity of one more NM proteins in said cells.

43. The method of claim 42 wherein the cells are retinal cells.

44. The method of claim 42 wherein the cells are recombinant host cells which are transfected with an expression construct which encodes said one or more NMs.

45. A method to identify candidate drugs for treating neuronal cell death, comprising:
contacting cells which express one or more neuronal marker (NM) genes selected from the group consisting of Acetylcholine receptor alpha 5; Nerve growth factor receptor, fast; Rat insulin-like growth factor binding protein (rIGFBP-6) mRNA, complete cds.; transforming growth factor, beta receptor I; taurine/beta-alanine transporter; Rat mRNA for proteasome subunit RC10-II, complete cds.; C holinergic receptor, nicotinic, alpha polypeptide 7 (neuronal nicotinic acetycholine receptor alpha 7) (bungarotoxin alpha); 6-phosphofructo-2kinase/fructose-2,6-biphosphatase 4; heterogeneous nuclear ribonucleoproteins methyltransferase-like 2 (S. cerevisiae); R. rattus mRNA for epididymal secretory glutathione peroxidase.; matrix metalloproteinase 14, membrane-inserted; cAMP response element binding protein; Solute carrier family 2 A3 (neuron glucose transporter); ATPase, Na+K+ transporting, alpha 1 polypeptide; Fyn proto-oncogene; protein kinase inhibitor, alpha; Rattus norvegicus galactosyltransferase associated kinase (GTA) mRNA, complete cds; Early growth response 1; Glutathione-S-transferase, placental enzyme pi type; neogenin; ATP synthase, H+ transporting, mitochondrial F0 complex, subunit c (subunit 9), isoform 1; 36 kDa calcium-dependent phospholipid-binding protein; This sequence comes from FIG. 1; conceptual translation differs that in published reference; calpactin 1; annexin II=36 kDa calcium-dependent phospholipid-binding protein [rats, RBL-2H3 basophilic leukemia cells, mRNA, 1362 nt].; Murine leukemia viral (v-raf-1) oncogene homolog 1 (3611-MSV); Inhibitor of DNA binding 1, helix-loop-helix protein (splice variation); alternative splicing: see also D28754; Rat mRNA for cyclin dependent kinase 2-alpha.; Tyrosine 3-monooxygenase/tryptophan 5-monooxygenase activation protein, zeta polypeptide; Solute carrier family 25, member 5 (adenine nucleotid translocator 2, fibroblast isoform (ATP-ADP carrier protein)); Dopa decarboxylase (aromatic L-amino acid decarboxylase); cadherin 22; Rat thymidine kinase mRNA, 5′ end.; Solute carrier family18 (vesicular monoamine) member 1 (chromaffin granule amine transporter); mitogen-activated protein kinase 6; R. norvegicus mRNA for Cdk-activating kinase; ADP-ribosylation factor 2; mismatch repair protein; CD24 antigen; glutamate-cysteine ligase, modifier subunit; PDZ and LIM domain 1 (elfin); casein kinase II beta subunit; Inhibitor of DNA binding 3, dominant negative helix-loop-helix protein; Rattus norvegicus Sprague-Dawley lipid-binding protein mRNA, complete cds; Rat mRNA for cyclin D1, complete cds.; Proliferating cell nuclear antigen; bone morphogenetic protein 2; VGF nerve growth factor inducible; activity regulated cytoskeletal-associated protein; Fos-like antigen 1; Cyclin G1; taurine/beta-alanine transporter; Vesicle-associated membrane protein (synaptobrevin 2); unction plakoglobin; Inhibitor of DNA binding 3, dominant negative helix-loop-helix protein; Heat shock 27 kDa protein; Solute carrier family18 (vesicular monoamine) member 1 (chromaffin granule amine transporter); mitogen-activated protein kinase 6; Interleukin 6 signal transducer; Synaptophysin; latexin; Nerve growth factor receptor, fast; 36 kDa calcium-dependent phospholipid-binding protein; This sequence comes from FIG. 1; conceptual translation differs that in published reference; calpactin 1; annexin II=36 kDa calcium-dependent phospholipid-binding protein [rats, RBL-2H3 basophilic leukemia cells, mRNA, 1362 nt].; transcription factor AP-1 (AA 1-334); Rat c-jun oncogene mRNA for transcription factor AP-1.; B-cell translocation gene 1, anti-proliferativeputative anti-proliferative factor; glycoprotein hormones, alpha subunit; Adenomatosis polyposis coli; Rattus norvegicus jun-D gene, complete cds; R. rattus mRNA for heat shock protein 70.; solute carrier family 30 (zinc transporter), member 1 zinc transporter; Cathepsin L; eukaryotic initiation factor 5 (eIF-5); 3-hydroxy-3-methylglutaryl-Coenzyme A synthase 1; cysteine-rich protein 3; Solute carrier family 7 member A1 (amino acid transporter cationic 1); Cytochrom P450 Lanosterol 14 alpha-demethylase; myc box dependent interacting protein 1; plectin; ATPase, Ca++ transporting, plasma membrane 1; Rattus norvegicus Sprague-Dawley lipid-binding protein mRNA, complete cds; cyclin-dependent kinase inhibitor 1A (P21); Annexin V; bone morphogenetic protein 2; 6phosphofructo-2-kinase/fructose-2,6-biphosphatase 4; Tumor necrosis factor receptor superfamily, member 1a; ezrin; Pim-1 oncogene; Fos like antigen 2transcription factor; B-cell translocation gene 2, anti-proliferative; Rattus norvegicus RIN1 mRNA, complete cds; Rat brain glucose-transporter protein mRNA, complete cds; jun B proto-oncogene; VGF nerve growth factor inducible; Interleukin 2 receptor, beta chain; Early growth response 1; Rat mRNA for LDL-receptor; Rat mRNA for 53 kD polypeptide induced by growth factors (EGF) and oncogenes (H-ras; src; polyoma middle T); urinary plasminogen activator receptor 2urinary-type plasminogen activator receptor; Rat transformation-associated protein (34A) mRNA, complete cds; serine (or cysteine) proteinase inhibitor, clade E (nexin, plasminogen activator inhibitor type 1), member 1; Fos-like antigen 1; and activity regulated cytoskeletal-associated protein, with a test compound;
determining expression of said one or more N\M genes by hybridization of mRNA of said cells to a nucleic acid probe which is complementary to said mRNA; and
identifying a test compound as a candidate drug for treating neuronal cell death if it increases expression of said one or more NM genes.

46. The method of claim 45 wherein the cells are retinal cells.

47. The method of claim 45 wherein the cells are recombinant host cells which are transfected with an expression construct which encodes said one or more NMs.

48. A method to identify candidate drugs for treating neuronal cell death, comprising:
contacting cells which express one or more neuronal marker (NM) proteins selected from the group consisting of: Acetylcholine receptor alpha 5; Nerve growth factor receptor, fast; Rat insulin-like growth factor binding protein (rIGFBP-6) mRNA, complete cds.; transforming growth factor, beta receptor I; taurine/beta-alanine transporter; Rat mRNA for proteasome subunit RC10-II, complete cds.; C holinergic receptor, nicotinic, alpha polypeptide 7 (neuronal nicotinic acetycholine receptor alpha 7) (bungarotoxin alpha); 6-phosphofructo-2kinase/fructose-2,6-biphosphatase 4; heterogeneous nuclear ribonucleoproteins methyltransferase-like 2 (S. cerevisiae); R. rattus mRNA for epididymal secretory glutathione peroxidase.; matrix metalloproteinase 14, membrane-inserted; cAMP response element binding protein; Solute carrier family 2 A3 (neuron glucose transporter); ATPase, Na+K+ transporting, alpha 1 polypeptide; Fyn proto-oncogene; protein kinase inhibitor, alpha; Rattus norvegicus galactosyltransferase associated kinase (GTA) mRNA, complete cds; Early growth response 1; Glutathione-S-transferase, placental enzyme pi type; neogenin; ATP synthase, H+ transporting, mitochondrial F0 complex, subunit c (subunit 9), isoform 1; 36 kDa calcium-dependent phospholipid-binding protein; This sequence comes from FIG. 1; conceptual translation differs that in published reference; calpactin 1; annexin II=36 kDa calcium-dependent phospholipid-binding protein [rats, RBL-2H3 basophilic leukemia cells, mRNA, 1362 nt].; Murine leukemia viral (v-raf-1) oncogene homolog 1 (3611-MSV); Inhibitor of DNA binding 1, helix-loop-helix protein (splice variation); alternative splicing: see also D28754; Rat mRNA for cyclin dependent kinase 2-alpha.; Tyrosine 3-monooxygenase/tryptophan 5-monooxygenase activation protein, zeta polypeptide; Solute carrier family 25, member 5 (adenine nucleotid translocator 2, fibroblast isoform (ATP-ADP carrier protein)); Dopa decarboxylase (aromatic L-amino acid decarboxylase); cadherin 22; Rat thymidine kinase mRNA, 5′ end.; Solute carrier family18 (vesicular monoamine) member 1 (chromaffin granule amine transporter); mitogen-activated protein kinase 6; R. norvegicus mRNA for Cdk-activating kinase; ADP-ribosylation factor 2; mismatch repair protein; CD24 antigen; glutamate-cysteine ligase, modifier subunit; PDZ and LIM domain 1 (elfin); casein kinase II beta subunit; Inhibitor of DNA binding 3, dominant negative helix-loop-helix protein; Rattus norvegicus Sprague-Dawley lipid-binding protein mRNA, complete cds; Rat mRNA for cyclin D1, complete cds.; Proliferating cell nuclear antigen; bone morphogenetic protein 2; VGF nerve growth factor inducible; activity regulated cytoskeletal-associated protein; Fos-like antigen 1; Cyclin G1; taurine/beta-alanine transporter; Vesicle-associated membrane protein (synaptobrevin 2); unction plakoglobin; Inhibitor of DNA binding 3, dominant negative helix-loop-helix protein; Heat shock 27 kDa protein; Solute carrier family18 (vesicular monoamine) member 1 (chromaffin granule amine transporter); mitogen-activated protein kinase 6; Interleukin 6 signal transducer; Synaptophysin; latexin; Nerve growth factor receptor, fast; 36 kDa calcium-dependent phospholipid-binding protein; This sequence comes from FIG. 1; conceptual translation differs that in published reference; calpactin 1; annexin II=36 kDa calcium-dependent phospholipid-binding protein [rats, RBL-2H3 basophilic leukemia cells, mRNA, 1362 nt].; transcription factor AP-1 (AA 1-334); Rat c-jun oncogene mRNA for transcription factor AP-1.; B-cell translocation gene 1, anti-proliferativeputative anti-proliferative factor; glycoprotein hormones, alpha subunit; Adenomatosis polyposis coli; Rattus norvegicus jun-D gene, complete cds; R. rattus mRNA for heat shock protein 70.; solute carrier family 30 (zinc transporter), member 1 zinc transporter; Cathepsin L; eukaryotic initiation factor 5 (eIF-5); 3-hydroxy-3-methylglutaryl-Coenzyme A synthase 1; cysteine-rich protein 3; Solute carrier family 7 member A1 (amino acid transporter cationic 1); Cytochrom P450 Lanosterol 14 alpha-demethylase; myc box dependent interacting protein 1; plectin; ATPase, Ca++ transporting, plasma membrane 1; Rattus norvegicus Sprague-Dawley lipid-binding protein mRNA, complete cds; cyclin-dependent kinase inhibitor 1A (P21); Annexin V; bone morphogenetic protein 2; 6phosphofructo-2-kinase/fructose-2,6-biphosphatase 4; Tumor necrosis factor receptor superfamily, member 1 a; ezrin; Pim-1 oncogene; Fos like antigen 2transcription factor; B-cell translocation gene 2, anti-proliferative; Rattus norvegicus RIN1 mRNA, complete cds; Rat brain glucose-transporter protein mRNA, complete cds; jun B proto-oncogene; VGF nerve growth factor inducible; Interleukin 2 receptor, beta chain; Early growth response 1; Rat mRNA for LDL-receptor; Rat mRNA for 53 kD polypeptide induced by growth factors (EGF) and oncogenes (H-ras; src; polyoma middle T); urinary plasminogen activator receptor 2urinary-type plasminogen activator receptor; Rat transformation-associated protein (34A) mRNA, complete cds; serine (or cysteine) proteinase inhibitor, clade E (nexin, plasminogen activator inhibitor type 1), member 1; Fos-like antigen 1; and activity regulated cytoskeletal-associated protein, with a test compound;
determining amount of said one or more NM proteins in said cells; and
identifying a test compound as a candidate drug for treating neuronal cell death if it increases the amount of one more NM proteins in said cells.

49. The method of claim 48 wherein the cells are retinal cells.

50. The method of claim 48 wherein the cells are recombinant host cells which are transfected with an expression construct which encodes said one or more NMs.

51. A method to identify candidate drugs for treating neuronal cell death, comprising:
contacting cells which express one or more neuronal marker (NM) proteins selected from the group consisting of: Acetylcholine receptor alpha 5; Nerve growth factor receptor, fast; Rat insulin-like growth factor binding protein (rIGFBP-6) mRNA, complete cds.; transforming growth factor, beta receptor I; taurine/beta-alanine transporter; Rat mRNA for proteasome subunit RC10-II, complete cds.; C holinergic receptor, nicotinic, alpha polypeptide 7 (neuronal nicotinic acetycholine receptor alpha 7) (bungarotoxin alpha); 6-phosphofructo-2kinase/fructose-2,6-biphosphatase 4; heterogeneous nuclear ribonucleoproteins methyltransferase-like 2 (S. cerevisiae); R. rattus mRNA for epididymal secretory glutathione peroxidase.; matrix metalloproteinase 14, membrane-inserted; cAMP response element binding protein; Solute carrier family 2 A3 (neuron glucose transporter); ATPase, Na+K+ transporting, alpha 1 polypeptide; Fyn proto-oncogene; protein kinase inhibitor, alpha; Rattus norvegicus galactosyltransferase associated kinase (GTA) mRNA, complete cds; Early growth response 1; Glutathione-S-transferase, placental enzyme pi type; neogenin; ATP synthase, H+ transporting, mitochondrial F0 complex, subunit c (subunit 9), isoform 1; 36 kDa calcium-dependent phospholipid-binding protein; This sequence comes from FIG. 1; conceptual translation differs that in published reference; calpactin 1; annexin II=36 kDa calcium-dependent phospholipid-binding protein [rats, RBL-2H3 basophilic leukemia cells, mRNA, 1362 nt].; Murine leukemia viral (v-raf-1) oncogene homolog 1 (3611-MSV); Inhibitor of DNA binding 1, helix-loop-helix protein (splice variation); alternative splicing: see also D28754; Rat mRNA for cyclin dependent kinase 2-alpha.; Tyrosine 3-monooxygenase/tryptophan 5-monooxygenase activation protein, zeta polypeptide; Solute carrier family 25, member 5 (adenine nucleotid translocator 2, fibroblast isoform (ATP-ADP carrier protein)); Dopa decarboxylase (aromatic L-amino acid decarboxylase); cadherin 22; Rat thymidine kinase mRNA, 5′ end.; Solute carrier family18 (vesicular monoamine) member 1 (chromaffin granule amine transporter); mitogen-activated protein kinase 6; R. norvegicus mRNA for Cdk-activating kinase; ADP-ribosylation factor 2; mismatch repair protein; CD24 antigen; glutamate-cysteine ligase, modifier subunit; PDZ and LIM domain 1 (elfin); casein kinase II beta subunit; Inhibitor of DNA binding 3, dominant negative helix-loop-helix protein; Rattus norvegicus Sprague-Dawley lipid-binding protein mRNA, complete cds; Rat mRNA for cyclin D1, complete eds.; Proliferating cell nuclear antigen; bone morphogenetic protein 2; VGF nerve growth factor inducible; activity regulated cytoskeletal-associated protein; Fos-like antigen 1; Cyclin G1; taurine/beta-alanine transporter; Vesicle-associated membrane protein (synaptobrevin 2); unction plakoglobin; Inhibitor of DNA binding 3, dominant negative helix-loop-helix protein; Heat shock 27 kDa protein; Solute carrier family18 (vesicular monoamine) member 1 (chromaffin granule amine transporter); mitogen-activated protein kinase 6; Interleukin 6 signal transducer; Synaptophysin; latexin; Nerve growth factor receptor, fast; 36 kDa calcium-dependent phospholipid-binding protein; This sequence comes from FIG. 1; conceptual translation differs that in published reference; calpactin 1; annexin II=36 kDa calcium-dependent phospholipid-binding protein [rats, RBL-2H3 basophilic leukemia cells, mRNA, 1362 nt].; transcription factor AP-1 (AA 1-334); Rat c-jun oncogene mRNA for transcription factor AP-1.; B-cell translocation gene 1, anti-proliferativeputative anti-proliferative factor; glycoprotein hormones, alpha subunit; Adenomatosis polyposis coli; Rattus norvegicus jun-D gene, complete cds; R. rattus mRNA for heat shock protein 70.; solute carrier family 30 (zinc transporter), member 1 zinc transporter; Cathepsin L; eukaryotic initiation factor 5 (eIF-5); 3-hydroxy-3-methylglutaryl-Coenzyme A synthase 1; cysteine-rich protein 3; Solute carrier family 7 member A1 (amino acid transporter cationic 1); Cytochrom P450 Lanosterol 14 alpha-demethylase; myc box dependent interacting protein 1; plectin; ATPase, Ca++ transporting, plasma membrane 1; Rattus norvegicus Sprague-Dawley lipid-binding protein mRNA, complete cds; cyclin-dependent kinase inhibitor 1A (P21); Annexin V; bone morphogenetic protein 2; 6phosphofructo-2-kinase/fructose-2,6-biphosphatase 4; Tumor necrosis factor receptor superfamily, member 1a; ezrin; Pim-1 oncogene; Fos like antigen 2transcription factor; B-cell translocation gene 2, anti-proliferative; Rattus norvegicus RIN1 mRNA, complete cds; Rat brain glucose-transporter protein mRNA, complete cds; jun B proto-oncogene; VGF nerve growth factor inducible; Interleukin 2 receptor, beta chain; Early growth response 1; Rat mRNA for LDL-receptor; Rat mRNA for 53 kD polypeptide induced by growth factors (EGF) and oncogenes (H-ras; src; polyoma middle T); urinary plasminogen activator receptor 2urinary-type plasminogen activator receptor; Rat transformation-associated protein (34A) mRNA, complete cds; serine (or cysteine) proteinase inhibitor, clade E (nexin, plasminogen activator inhibitor type 1), member 1; Fos-like antigen 1; and activity regulated cytoskeletal-associated protein, with a test compound;
determining activity of said one or more NM proteins in said cells; and
identifying a test compound as a candidate drug for treating neuronal cell death if it increases the activity of one more NM proteins in said cells.

52. The method of claim 51 wherein the cells are retinal cells.

53. The method of claim 51 wherein the cells are recombinant host cells which are transfected with an expression construct which encodes said one or more NMs.
